(19) United States
(12) Patent Application Publication
(10) Pub. No.: US 2008/0070868 A1
(43) Pub. Date: Mar. 20, 2008

(54) Novel Thiazole Inhibitors of Fructose 1,6-bisphosphatase

(75) Inventors: Qun Dang, San Diego, CA (US); Joseph J. Kopcho, San Diego, CA (US); Scott J. Hecker, Del Mar, CA (US); Bheemarao G. Ugarkar, Bangalore (IN)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Metabasis Therapeutics, Inc., La Jolla, CA (US)

(21) Appl. No.: 11/841,840

(22) Filed: Aug. 20, 2007

Related U.S. Application Data
(63) Continuation of application No. 11/660,169, filed on Apr. 4, 2007.
(60) Provisional application No. 60/602,518, filed on Aug. 18, 2004.
(60) Provisional application No. 60/662,138, filed on Mar. 15, 2005.

(51) Int. Cl. A61K 31/675; A61P 3/08; C07F 9/02

(52) U.S. Cl. 514/92; 548/112

(57) ABSTRACT

Compounds of Formula I, their prodrugs and salts, their preparation and their uses are described.

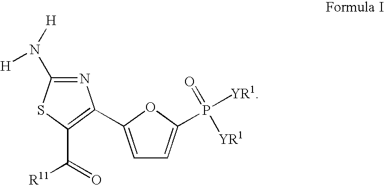

### CROSS-REFERENCE TO RELATED APPLICATIONS

This application is a continuation of U.S. application Ser. No. 11/660,169, filed Feb. 12, 2007, which is the national stage of international application No. PCT/US2005/029176, filed Aug. 18, 2005, which claims priority from U.S. Provisional Application No. 60/602,518, filed Aug. 18, 2004, and U.S. Provisional Application No. 60/662,138, filed Mar. 15, 2005, each of which is hereby incorporated by reference in its entirety.

### BACKGROUND OF THE INVENTION

1. Field of the Invention

The present invention is directed towards novel phosphorus-containing 5-ketothiazole compounds that are potent inhibitors of fructose 1,6-bisphosphatase (FBPase). In one aspect, the invention is directed toward phosphonic acids and prodrugs thereof. In another aspect, the present invention is directed to the preparation and the clinical use of these FBPase inhibitors as a method of treatment or prevention of diseases responsive to inhibition of gluconeogenesis and in diseases responsive to lower blood glucose levels.

The compounds are also useful in treating or preventing excess glycogen storage diseases and diseases such as cardiovascular diseases including atherosclerosis, myocardial ischemic injury, and diseases such as metabolic disorders such as hypercholesterolemia, hyperlipidemia which are exacerbated by hyperinsulinema and hyperglycemia.

The invention also comprises the novel compounds, methods of making them and methods of using them as specified below in Formula I.

2. Background Art

The following description of the background of the invention is provided to aid in understanding the invention, but is not admitted to be, or to describe, prior art to the invention. All cited publications are incorporated by reference in their entirety.

Diabetes mellitus (or diabetes) is one of the most prevalent diseases in the world today. Diabetic patients have been divided into two classes, namely type I or insulin-dependent diabetes mellitus and type II diabetes mellitus (T2DM). T2DM accounts for approximately 90% of all diabetics and is estimated to affect 12-14 million adults in the U.S. alone (6.6% of the population). T2DM is characterized by both fasting hyperglycemia and exaggerated postprandial increases in plasma glucose levels. T2DM is associated with a variety of long-term complications, including microvascular diseases such as retinopathy, nephropathy and neuropathy, and macrovascular diseases such as coronary heart disease. Numerous studies in animal models demonstrate a causal relationship between long term hyperglycemia and complications. Results from the Diabetes Control and Complications Trial (DCCT) and the Stockholm Prospective Study demonstrate this relationship for the first time in man by showing that insulin-dependent diabetics with tighter glycemic control are at substantially lower risk for the development and progression of these complications. Tighter control is also expected to benefit T2DM patients.

Gluconeogenesis from pyruvate and other 3-carbon precursors is a highly regulated biosynthetic pathway requiring eleven enzymes. Seven enzymes catalyze reversible reactions and are common to both gluconeogenesis and glycolysis. Four enzymes catalyze reactions unique to gluconeogenesis, namely pyruvate carboxylase, phosphoenolpyruvate carboxykinase, fructose-1,6-bisphosphatase and glucose-6-phosphatase. Overall flux through the pathway is controlled by the specific activities of these enzymes, the enzymes that catalyzed the corresponding steps in the glycolytic direction, and by substrate availability. Dietary factors (glucose, fat) and hormones (insulin, glucagon, glucocorticoids, epinephrine) coordinatively regulate enzyme activities in the gluconeogenesis and glycolysis pathways through gene expression and post-translational mechanisms.

Synthetic inhibitors of FBPase have also been reported. McNiel reported that fructose-2,6-bisphosphate analogs inhibit FBPase by binding to the substrate site. J. Am. Chem. Soc., 106:7851-7853 (1984); U.S. Pat. No. 4,968,790 (1984). These compounds, however, were relatively weak and did not inhibit glucose production in hepatocytes presumably due to poor cell penetration.

Gruber reported that some nucleosides can lower blood glucose in the whole animal through inhibition of FBPase. These compounds exert their activity by first undergoing phosphorylation to the corresponding monophosphate. EP 0 427 799 B1.

Gruber et al. U.S. Pat. No. 5,658,889 described the use of inhibitors of the AMP site of FBPase to treat diabetes. WO 98/39344, WO/39343, WO 98/39342, U.S. Pat. No. 6,489,476, and U.S. 2002/0173490 describe specific inhibitors of FBPase to treat diabetes.

### BRIEF DESCRIPTION OF THE DRAWINGS/FIGURES

FIG. 1. Depicts blood glucose lowering in fasting ZDF rats following oral administration of compounds 4.6 or 2.1 at 10 mg/kg in polyethylene glycol-400.

FIG. 2. Depicts blood glucose lowering in fasting ZDF rats following oral administration of compound 2.1 at doses ranging from 10 to 300 mg/kg. Animals were refed 9 h after drug administration.

### BRIEF SUMMARY OF THE INVENTION

The present invention relates to compounds of Formula I and pharmaceutically acceptable salts and prodrugs thereof.

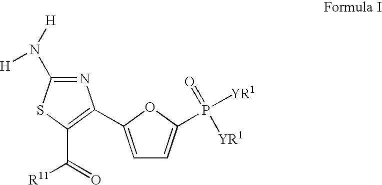

Also provided are methods for treating a disease or condition responsive to inhibition of gluconeogenesis or responsive to lowered blood glucose levels, the methods comprising the step of administering to an animal a therapeutically effective amount of a compound of Formula I, or pharmaceutically acceptable salts or prodrugs thereof.

Also provided are methods for treating diabetes, the methods comprising the step of administering to an animal a therapeutically effective amount of a compound of Formula I, or pharmaceutically acceptable salts or prodrugs thereof.

Also provided are methods for preventing diabetes, the methods comprising the step of administering to an animal at risk for developing diabetes a therapeutically effective amount of a compound of Formula I, or pharmaceutically acceptable salts or prodrugs thereof. In one aspect, an animal at risk for developing diabetes has a disease or condition selected from the group consisting of impaired glucose tolerance, insulin resistance, hyperglycemia, obesity, accelerated gluconeogenesis, and increased hepatic glucose output.

Also provided are methods for treating impaired glucose tolerance, the methods comprising the step of administering to an animal a therapeutically effective amount of a compound of Formula I, or pharmaceutically acceptable salts or prodrugs thereof.

Also provided are methods for treating insulin resistance, the methods comprising the step of administering to an animal a therapeutically effective amount of a compound of Formula I, or pharmaceutically acceptable salts or prodrugs thereof.

Also provided are methods for treating a disease or condition selected from the group consisting of hyperlipidemia, atherosclerosis, ischemic injury, and hypercholesterolemia, the methods comprising the step of administering to an animal a therapeutically effective amount of a compound of Formula I, or pharmaceutically acceptable salts or prodrugs thereof.

Also provided are methods for treating a glycogen storage disease, the methods comprising the step of administering to an animal a therapeutically effective amount of a compound of Formula I, or pharmaceutically acceptable salts or prodrugs thereof.

Also provided are pharmaceutical compositions comprising compounds of Formula I or pharmaceutically acceptable salts or prodrugs thereof and a pharmaceutically acceptable carrier.

Also provided are methods of synthesizing compounds of Formula I or pharmaceutically acceptable salts or prodrugs thereof.

### DEFINITIONS

In accordance with the present invention and as used herein, the following terms are defined with the following meanings, unless explicitly stated otherwise.

The term “alkyl” refers to saturated aliphatic groups including straight-chain, branched chain and cyclic groups, up to and including 20 carbon atoms. Suitable alkyl groups include methyl, ethyl, n-propyl, isopropyl, and cyclopropyl. The alkyl may be optionally substituted with 1-3 substituents.

The term “aryl” refers to aromatic groups which have 5-14 ring atoms and at least one ring having a conjugated pi electron system and includes carbocyclic aryl, heterocyclic aryl and biaryl groups, all of which may be optionally substituted. The aryl may be optionally substituted with 1-6 substituents.

Carbocyclic aryl groups are groups which have 6-14 ring atoms wherein the ring atoms on the aromatic ring are carbon atoms. Carbocyclic aryl groups include monocyclic carbocyclic aryl groups and polycyclic or fused compounds such as optionally substituted naphthyl groups.

Heterocyclic aryl or heteroaryl groups are groups which have 5-14 ring atoms wherein 1 to 4 heteroatoms are ring atoms in the aromatic ring and the remainder of the ring atoms being carbon atoms. Suitable heteroatoms include oxygen, sulfur, nitrogen, and selenium. Suitable heteroaryl groups include furanyl, thienyl, pyridyl, pyrrolyl, N-lower alkyl pyrrolyl, pyridyl-N-oxide, pyrimidyl, pyrazinyl, imidazolyl, and the like, all optionally substituted.

The term “monocyclic aryl” refers to aromatic groups which have 5-6 ring atoms and includes carbocyclic aryl and heterocyclic aryl. Suitable aryl groups include phenyl, furanyl, pyridyl, and thienyl. Aryl groups may be substituted. The term “bicyclic aryl” refers to aromatic groups which have 10-12 ring atoms and includes carbocyclic aryl and heterocyclic aryl. Suitable aryl groups include naphthyl. Aryl groups may be substituted.

The term “monocyclic heteroaryl” refers to aromatic groups which have 5-6 ring atoms wherein 1 to 4 heteroatoms are ring atoms in the aromatic ring and the remainder of the ring atoms being carbon atoms. Suitable heteroatoms include oxygen, sulfur, nitrogen, and selenium. The term “bicyclic heteroaryl” refers to aromatic groups which have 10-12 ring atoms wherein 1 to 4 heteroatoms are ring atoms in the aromatic ring and the remainder of the ring atoms being carbon atoms. Suitable heteroatoms include oxygen, sulfur, nitrogen, and selenium.

The term “biaryl” represents aryl groups which have 5-14 atoms containing more than one aromatic ring including both fused ring systems and aryl groups substituted with other aryl groups. Such groups may be optionally substituted. Suitable biaryl groups include naphthyl and biphenyl.

The term “optionally substituted” or “substituted” includes groups substituted by one to four substituents, independently selected from lower alkyl, lower aryl, lower aralkyl, lower cyclic alkyl, lower heterocycloalkyl, hydroxy, lower alkoxy, lower aryloxy, perhaloalkoxy, aralkoxy, lower heteroaryl, lower heteroaryloxy, lower heteroarylalkyl, lower heteroaralkoxy, azido, amino, halo, lower alkylthio, oxo, lower acylalkyl, lower carboxy esters, carboxyl, -carboxamido, nitro, lower acyloxy, lower aminoalkyl, lower alkylaminoaryl, lower alkylaryl, lower alkylaminoalkyl, lower alkoxyaryl, lower arylamino, lower aralkylamino, sulfonyl, lower-carboxamidoalkylaryl, lower-carboxamidoaryl, lower hydroxyalkyl, lower haloalkyl, lower alkylaminoalkylcarboxy-, lower aminocarboxamidoalkyl-, cyano, lower alkoxyalkyl, lower perhaloalkyl, and lower arylalkyloxyalkyl. “Substituted aryl” and “substituted heteroaryl” refers to aryl and heteroaryl groups substituted with 1-6 substituents. These substituents are selected from the group consisting of lower alkyl, lower alkoxy, lower perhaloalkyl, halo, hydroxy, and amino.

The term “-aralkyl” refers to an alkylene group substituted with an aryl group. Suitable aralkyl groups include benzyl, picolyl, and the like, and may be optionally substituted. The aryl portion may have 5-14 ring atoms and the alkyl portion may have up to and including 10 carbon atoms. “Heteroarylalkyl” refers to an alkylene group substituted with a heteroaryl group.

The term “alkylaryl-” refers to an aryl group substituted with an alkyl group. “Lower alkylaryl-” refers to such groups where alkyl is lower alkyl. The aryl portion may have 5-14 ring atoms and the alkyl portion may have up to and including 10 carbon atoms. The term “lower” referred to herein in connection with organic radicals or compounds respectively defines such as with up to and including 10, in one aspect up to and including 6, and in another aspect one to four carbon atoms. Such groups may be straight chain, branched, or cyclic.

The term “cyclic alkyl” or “cycloalkyl” refers to alkyl groups that are cyclic of 3 to 10 carbon atoms, and in one aspect are 3 to 6 carbon atoms. Suitable cyclic groups include norbornyl and cyclopropyl. Such groups may be substituted.

The term “heterocyclic,” “heterocyclic alkyl” or “heterocycloalkyl” refer to cyclic groups of 3 to 10 atoms, and in one aspect are 3 to 6 atoms, containing at least one heteroatom, in a further aspect are 1 to 3 heteroatoms. Suitable heteroatoms include oxygen, sulfur, and nitrogen. Heterocyclic groups may be attached through a nitrogen or through a carbon atom in the ring. The heterocyclic alkyl groups include unsaturated cyclic, fused cyclic and spirocyclic groups. Suitable heterocyclic groups include pyrrolidinyl, morpholino, morpholinoethyl, and pyridyl.

The terms “arylamino” (a), and “aralkylamino” (b), respectively, refer to the group —NRR′ wherein respectively, (a) R is aryl and R′ is hydrogen, alkyl, aralkyl, heterocycloalkyl, or aryl, and (b) R is aralkyl and R′ is hydrogen, aralkyl, aryl, alkyl or heterocycloalkyl.

The term “acyl” refers to —C(O)R where R is alkyl, heterocycloalkyl, or aryl. The term “lower acyl” refers to where R is lower alkyl. The term C₁-C₄ acyl refers to where R is C₁-C₄.

The term “carboxy esters” refers to —C(O)OR where R is alkyl, aryl, aralkyl, cyclic alkyl, or heterocycloalkyl, all optionally substituted.

The term “carboxyl” refers to —C(O)OH.

The term “oxo” refers to ═O in an alkyl or heterocycloalkyl group. In one aspect, the resulting aldehyde or ketone exists in a hydrated form of the structure —C(OH)₂—.

The term “amino” refers to —NRR′ where R and R′ are independently selected from hydrogen, alkyl, aryl, aralkyl and heterocycloalkyl, all except H are optionally substituted; and R and R′ can form a cyclic ring system.

The term “-carboxylamido” refers to —CONR₂ where each R is independently hydrogen or alkyl.

The term “-sulphonylamido” or “-sulfonylamido” refers to —S(═O)₂NR₂ where each R is independently hydrogen or alkyl.

The term “halogen” or “halo” refers to —F, —Cl, —Br and —I.

The term “alkylaminoalkylcarboxy” refers to the group alkyl-NR-alk-C(O)—O— where “alk” is an alkylene group, and R is a H or lower alkyl.

The term “sulphonyl” or “sulfonyl” refers to —SO₂R, where R is H, alkyl, aryl, aralkyl, or heterocycloalkyl.

The term “sulphonate” or “sulfonate” refers to —SO₂OR, where R is —H, alkyl, aryl, aralkyl, or heterocycloalkyl.

The term “alkenyl” refers to unsaturated groups which have 2 to 12 atoms and contain at least one carbon-carbon double bond and includes straight-chain, branched-chain and cyclic groups. Alkenyl groups may be optionally substituted. Suitable alkenyl groups include allyl. “1-alkenyl” refers to alkenyl groups where the double bond is between the first and second carbon atom. If the 1-alkenyl group is attached to another group, e.g. it is a W substituent attached to the cyclic phosphonate, it is attached at the first carbon.

The term “alkynyl” refers to unsaturated groups which have 2 to 12 atoms and contain at least one carbon-carbon triple bond and includes straight-chain, branched-chain and cyclic groups. Alkynyl groups may be optionally substituted. Suitable alkynyl groups include ethynyl. “1-alkynyl” refers to alkynyl groups where the triple bond is between the first and second carbon atom. If the 1-alkynyl group is attached to another group, e.g. it is a W substituent attached to the cyclic phosphonate, it is attached at the first carbon.

The term “alkylene” refers to a divalent straight chain, branched chain or cyclic saturated aliphatic group. In one aspect the alkylene group contains up to and including 10 atoms. In another aspect the alkylene chain contains up to and including 6 atoms. In a further aspect the alkylene groups contains up to and including 4 atoms. The alkylene group can be either straight, branched or cyclic. The alkylene may be optionally substituted with 1-3 substituents.

The term “acyloxy” refers to the ester group —O—C(O)R, where R is H, alkyl, alkenyl, alkynyl, aryl, aralkyl, or heterocycloalkyl.

The term “aminoalkyl-” refers to the group NR₂-alk- wherein “alk” is an alkylene group and R is selected from —H, alkyl, aryl, aralkyl, and heterocycloalkyl.

The term “alkylaminoalkyl-” refers to the group alkyl-NR-alk- wherein each “alk” is an independently selected alkylene, and R is H or lower alkyl. “Lower alkylaminoalkyl-” refers to groups where the alkyl and the alkylene group is lower alkyl and alkylene, respectively.

The term “arylaminoalkyl-” refers to the group aryl-NR-alk- wherein “alk” is an alkylene group and R is —H, alkyl, aryl, aralkyl, or heterocycloalkyl. In “lower arylaminoalkyl-,” the alkylene group is lower alkylene.

The term “alkylaminoaryl-” refers to the group alkyl-NR-aryl- wherein “aryl” is a divalent group and R is —H, alkyl, aralkyl, or heterocycloalkyl. In “lower alkylaminoaryl-,” the alkyl group is lower alkyl.

The term “alkoxyaryl-” refers to an aryl group substituted with an alkyloxy group. In “lower alkyloxyaryl-”, the alkyl group is lower alkyl.

The term “aryloxyalkyl-” refers to an alkyl group substituted with an aryloxy group.

The term “aralkyloxyalkyl-” refers to the group aryl-alk-O-alk- wherein “alk” is an alkylene group. “Lower aralkyloxyalkyl-” refers to such groups where the alkylene groups are lower alkylene.

The term “alkoxy-” or “alkyloxy-” refers to the group alkyl-O—.

The term “alkoxyalkyl-” or “alkyloxyalkyl-” refer to the group alkyl-O-alk- wherein “alk” is an alkylene group. In “lower alkoxyalkyl-,” each alkyl and alkylene is lower alkyl and alkylene, respectively.

The term “alkylthio-” refers to the group alkyl-S—.

The term “alkylthioalkyl-” refers to the group alkyl-5-alk- wherein “alk” is an alkylene group. In “lower alkylthioalkyl-” each alkyl and alkylene is lower alkyl and alkylene, respectively.

The term “alkoxycarbonyloxy-” refers to alkyl-O—C(O)—O—.

The term “aryloxycarbonyloxy-” refers to aryl-O—C(O)—O—.

The term “alkylthiocarbonyloxy-” refers to alkyl-S—C(O)—O—.

The term “amido” refers to the NR₂ group next to an acyl or sulfonyl group as in NR₂—C(O)—, RC(O)—NR¹—, NR₂—S(═O)₂— and RS(═O)₂—NR¹—, where R and R¹ include —H, alkyl, aryl, aralkyl, and heterocycloalkyl.

The term “carboxamido” refers to NR₂—C(O)— and RC(O)—NR¹—, where R and R¹ include —H, alkyl, aryl, aralkyl, and heterocycloalkyl. The term does not include urea, —NR—C(O)—NR—.

The terms “sulphonamido” or “sulfonamido” refer to NR₂—S(═O)₂— and RS(═O)₂—NR¹—, where R and R¹ include —H, alkyl, aryl, aralkyl, and heterocycloalkyl. The term does not include sulfonylurea, —NR—S(═O)₂—NR—.

The terms “carboxamidoalkylaryl” and “carboxamidoaryl” refer to an aryl-alk-NR¹—C(O), and ar-NR¹—C(O)-alk-, respectively where “ar” is aryl, “alk” is alkylene, R¹ and R include H, alkyl, aryl, aralkyl, and heterocycloalkyl.

The terms “sulfonamidoalkylaryl” and “sulfonamidoaryl” refer to an aryl-alk-NR¹—S(═O)₂—, and ar-NR¹—S(═O)₂—, respectively where “ar” is aryl, “alk” is alkylene, R¹ and R include —H, alkyl, aryl, aralkyl, and heterocycloalkyl.

The term “hydroxyalkyl” refers to an alkyl group substituted with one —OH.

The term “haloalkyl” refers to an alkyl group substituted with one halo.

The term “cyano” refers to —C≡N.

The term “nitro” refers to —NO₂.

The term “acylalkyl” refers to an alkyl-C(O)-alk-, where “alk” is alkylene.

The term “aminocarboxamidoalkyl-” refers to the group NR₂—C(O)—N(R)-alk- wherein R is an alkyl group or H and “alk” is an alkylene group. “Lower aminocarboxamidoalkyl-” refers to such groups wherein “alk” is lower alkylene.

The term “heteroarylalkyl” refers to an alkylene group substituted with a heteroaryl group.

The term “perhalo” refers to groups wherein every C—H bond has been replaced with a C-halo bond on an aliphatic or aryl group. Suitable perhaloalkyl groups include —CF₃ and —CFCl₂.

The phrase “therapeutically effective amount” means an amount of a compound or a combination of compounds that ameliorates, attenuates or eliminates one or more of the symptoms of a particular disease or condition or prevents, modifies, or delays the onset of one or more of the symptoms of a particular disease or condition.

The term “pharmaceutically acceptable salt” includes salts of compounds of Formula I and its prodrugs derived from the combination of a compound of this invention and an organic or inorganic acid or base. Suitable acids include acetic acid, adipic acid, benzenesulfonic acid, (+)-7,7-dimethyl-2-oxobicyclo[2.2.1]heptane-1-methanesulfonic acid, citric acid, 1,2-ethanedisulfonic acid, dodecyl sulfonic acid, fumaric acid, glucoheptonic acid, gluconic acid, glucuronic acid, hippuric acid, hydrochloride hemiethanolic acid, HBr, HCl, HI, 2-hydroxyethanesulfonic acid, lactic acid, lactobionic acid, maleic acid, methanesulfonic acid, methylbromide acid, methyl sulfuric acid, 2-naphthalenesulfonic acid, nitric acid, oleic acid, 4,4′-methylenebis [3-hydroxy-2-naphthalenecarboxylic acid], phosphoric acid, polygalacturonic acid, stearic acid, succinic acid, sulfuric acid, sulfosalicylic acid, tannic acid, tartaric acid, terphthalic acid, and p-toluenesulfonic acid.

The term “patient” refers to an animal being treated including a mammal, such as a dog, a cat, a cow, a horse, a sheep, and a human. Another aspect includes a mammal, both male and female.

The term “prodrug” as used herein refers to any compound that when administered to a biological system generates a biologically active compound as a result of spontaneous chemical reaction(s), enzyme catalyzed chemical reaction(s), and/or metabolic chemical reaction(s), or a combination of each. Standard prodrugs are formed using groups attached to functionality, e.g. HO—, HS—, HOOC—, R₂N—, associated with the drug, that cleave in vivo. Standard prodrugs include but are not limited to carboxylate esters where the group is alkyl, aryl, aralkyl, acyloxyalkyl, alkoxycarbonyloxyalkyl as well as esters of hydroxyl, thiol and amines where the group attached is an acyl group, an alkoxycarbonyl, aminocarbonyl, phosphate or sulfate. The groups illustrated are exemplary, not exhaustive, and one skilled in the art could prepare other known varieties of prodrugs. Such prodrugs of the compounds of Formula I fall within this scope. Prodrugs must undergo some form of a chemical transformation to produce the compound that is biologically active or is a precursor of the biologically active compound. In some cases, the prodrug is biologically active, usually less than the drug itself, and serves to improve drug efficacy or safety through improved oral bioavailability, pharmacodynamic half-life, etc. Prodrug forms of compounds may be utilized, for example, to improve bioavailability, improve subject acceptability such as by masking or reducing unpleasant characteristics such as bitter taste or gastrointestinal irritability, alter solubility such as for intravenous use, provide for prolonged or sustained release or delivery, improve ease of formulation, or provide site-specific delivery of the compound. Prodrugs are described in The Organic Chemistry of Drug Design and Drug Action, by Richard B. Silverman, Academic Press, San Diego, 1992. Chapter 8: “Prodrugs and Drug delivery Systems” pp. 352-401; Design of Prodrugs, edited by H. Bundgaard, Elsevier Science, Amsterdam, 1985; Design of Biopharmaceutical Properties through Prodrugs and Analogs, Ed. by E. B. Roche, American Pharmaceutical Association, Washington, 1977; and Drug Delivery Systems, ed. by R. L. Juliano, Oxford Univ. Press, Oxford, 1980.

The structure

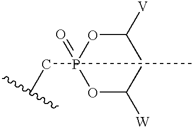

has a plane of symmetry running through the phosphorus-oxygen double bond when V═W and V and W are either both pointing up or both pointing down.

The term “cyclic phosphonate ester of 1,3-propanediol”, “cyclic phosphonate diester of 1,3-propanediol,” “2 oxo 2λ^{5[}1,3,2] dioxaphosphorinane”, “2-oxo-[1,3,2]-dioxaphosphorinane,” or “dioxaphosphorinane” refers to the following:

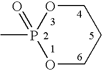

The phrase “together V and Z are connected via an additional 3-5 atoms to form a cyclic group, optionally containing one heteroatom, that is fused to an aryl group attached at the beta and gamma position to the O attached to the phosphorus” includes the following:

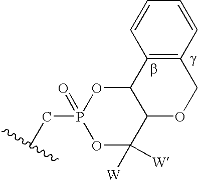

As shown above together V and Z are connected via 4 additional atoms.

The phrase “together W and W′ are connected via an additional 2-5 atoms to form a cyclic group, optionally containing 0-2 heteroatoms, and V must be aryl, substituted aryl, heteroaryl, or substituted heteroaryl” includes the following:

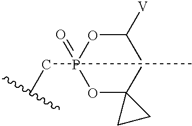

As shown above together W and W′ are connected via an additional 2 atoms.

The structure above has V=aryl, and a spiro-fused cyclopropyl group for W and W′.

The term “cyclic phosphonate” refers to

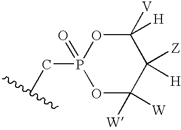

The carbon attached to V must have a C—H bond. The carbon attached to Z must also have a C—H bond.

The term “cis” stereochemistry refers to the spatial relationship of the V group and the substituent attached to the phosphorus atom via an exocyclic single bond on the six membered 2-oxo-phosphorinane ring. The structures A and B below show two possible cis-isomers of 2- and 4-substituted 2-oxo-phosphorinane. Structure A shows cis-isomer of (2S,4R)-configuration whereas structure B shows cis-isomer of (2R,4S)-configuration.

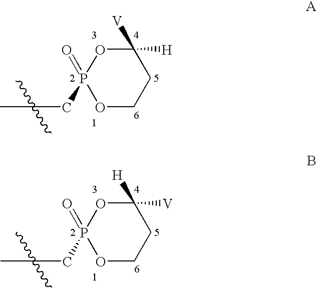

The term “trans” stereochemistry refers to the spatial relationship of the V group and the substituent attached to the phosphorus atom via an exocyclic single bond on the six membered 2-oxo-phosphorinane ring. The structures C and D below show two possible trans-isomers of 2- and 4-substituted 2-oxo-phosphorinane. Structure C shows trans-isomer of (2S,4S)-configuration whereas structure D shows trans-isomer of (2R,4R)-configuration.

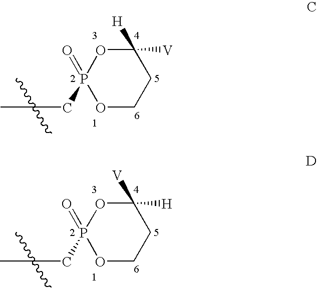

The term “percent enantiomeric excess (% ee)” refers to optical purity. It is obtained by using the following formula:

$$\frac{[ R ] - [ S ]}{[ R ] + [ S ]} \times 100 = \% R - \% S$$

where [R] is the amount of the R isomer and [S] is the amount of the S isomer. This formula provides the % ee when R is the dominant isomer.

The term “enantioenriched” or “enantiomerically enriched” refers to a sample of a chiral compound that consists of more of one enantiomer than the other. The extent to which a sample is enantiomerically enriched is quantitated by the enantiomeric ratio or the enantiomeric excess.

The term “enhanced oral bioavailability” refers to an increase of at least 50% of the absorption of the dose of the parent drug. In an additional aspect the increase in oral bioavailability of the prodrug (compared to the parent drug) is at least 100%, that is a doubling of the absorption. Measurement of oral bioavailability usually refers to measurements of the prodrug, drug, or drug metabolite in blood, plasma, tissues, or urine following oral administration compared to measurements following parenteral administration.

The term “therapeutic index” refers to the ratio of the dose of a drug or prodrug that produces a therapeutically beneficial response relative to the dose that produces an undesired response such as death, an elevation of markers that are indicative of toxicity, and/or pharmacological side effects.

The term “sustained delivery” refers to an increase in the period in which there is a prolongation of therapeutically-effective drug levels due to the presence of the prodrug.

The term “bypassing drug resistance” refers to the loss or partial loss of therapeutic effectiveness of a drug (drug resistance) due to changes in the biochemical pathways and cellular activities important for producing and maintaining the biological activity of the drug and the ability of an agent to bypass this resistance through the use of alternative pathways or the failure of the agent to induce changes that tend to resistance.

The terms “treating” or “treatment” of a disease includes preventing the disease from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease).

### DETAILED DESCRIPTION OF THE INVENTION

The present invention relates to compounds of Formula I, and pharmaceutically acceptable salts and prodrugs thereof as represented by Formula I:

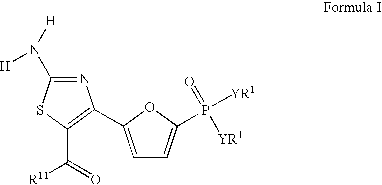

wherein:

R¹¹ is selected from the group consisting of C₁-C₂₀ alkyl, C₁-C₂₀ cycloalkyl, monocyclic aryl, bicyclic aryl, monocyclic heteroaryl and bicyclic heteroaryl, optionally substituted with halogen, OH, C₁-C₄ alkoxy, cyano, alkyl, aryl, NR³₂, NR⁴₂, morpholino, pyrrolidinyl, NMe₂ and perhaloalkyl;

Y is independently selected from the group consisting of —O—, and —NR⁶—;

when Y is —O—, then R¹ attached to —O— is independently selected from the group consisting of —H, optionally substituted aryl, optionally substituted-alkylaryl, —C(R²)₂OC(O)NR²₂, —NR²—C(O)—R³, —C(R²)₂—OC(O)R³, —C(R²)₂—O—C(O)OR³, —C(R²)₂OC(O)SR³, -alkyl-S—C(O)OR³; and -alkyl-S—C(O)R³;

when Y is —NR⁶—, then R¹ attached to —NR⁶— is independently selected from the group consisting of —H, —[C(R²)₂]_{q}—COOR³, —C(R⁴)₂COOR³,

—[C(R²)₂]_{q}—C(O)SR, and -cycloalkylene-COOR³;

or when one Y—R¹ is —NR¹⁵(R¹⁶) then the other Y—R¹ is —N(R¹⁸)—(CR¹²R¹³)ₙ—C(O)—R¹⁴;

or both Y—R¹ are —N(R¹⁸)—(CR¹²R¹³)ₙ—C(O)—R¹⁴;

or when either Y is independently selected from —O— and —NR⁶—, then together R¹ and R¹ are

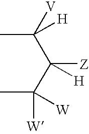

wherein

V, W, and W′ are independently selected from the group consisting of hydrogen, optionally substituted alkyl, optionally substituted aralkyl, heterocycloalkyl, aryl, substituted aryl, heteroaryl, substituted heteroaryl, optionally substituted 1-alkenyl, and optionally substituted 1-alkynyl; or

together V and Z are connected via an additional 3-5 atoms to form a cyclic group containing 5-7 atoms, optionally 1 heteroatom, substituted with hydroxy, acyloxy, alkoxycarbonyloxy, or aryloxycarbonyloxy attached to a carbon atom that is three atoms from both Y groups attached to the phosphorus; or

together V and Z are connected via an additional 3-5 atoms to form a cyclic group, optionally containing 1 heteroatom, that is fused to an aryl group at the beta and gamma position to the Y attached to the phosphorus; or

together V and W are connected via an additional 3 carbon atoms to form an optionally substituted cyclic group containing 6 carbon atoms and substituted with one substituent selected from the group consisting of hydroxy, acyloxy, alkoxycarbonyloxy, alkylthiocarbonyloxy, and aryloxycarbonyloxy, attached to one of said carbon atoms that is three atoms from a Y attached to the phosphorus; or

together Z and W are connected via an additional 3-5 atoms to form a cyclic group, optionally containing one heteroatom, and V must be aryl, substituted aryl, heteroaryl, or substituted heteroaryl; or

together W and W′ are connected via an additional 2-5 atoms to form a cyclic group, optionally containing 0-2 heteroatoms, and V must be aryl, substituted aryl, heteroaryl, or substituted heteroaryl;

Z is selected from the group consisting of —CHR²OH, —CHR²OC(O)R³, —CHR²OC(S)R³, —CHR²OC(S)OR³, —CHR²OC(O)SR³, —CHR²OCO₂R³, —OR², —SR², —CHR²N₃, —CH₂aryl, —CH(aryl)OH, —CH(CH═CR²₂)OH, —CH(C≡CR²)OH, —R², —NR₂, —OCOR³, —OCO₂R³, —SCOR³, —SCO₂R³, —NHCOR², —NHCO₂R³, —CH₂NHaryl, —(CH₂)ₚ—OR², and —(CH₂)ₚ—SR;
n is an integer from 1 to 3;
p is an integer 2 or 3;
q is an integer 1 or 2;
with the provisos that:

a) V, Z, W, W′ are not all —H; and

b) when Z is —R², then at least one of V, W, and W′ is not —H, alkyl, aralkyl, or heterocycloalkyl;

R² is selected from the group consisting of R³ and —H;

R³ is selected from the group consisting of alkyl, aryl, heterocycloalkyl, and aralkyl;

each R⁴ is independently selected from the group consisting of —H and alkyl, or together R⁴ and R⁴ form a cyclic alkyl group;

R⁶ is selected from the group consisting of —H, lower alkyl, acyloxyalkyl, alkoxycarbonyloxyalkyl, and lower acyl;

each R¹² and R¹³ is independently selected from the group consisting of H, lower alkyl, lower aryl, and lower aralkyl, all optionally substituted, or R¹² and R¹³ together are connected via 2-6 atoms, optionally including 1-2 heteroatoms selected from the group consisting of O, N and S, to form a cyclic group;

each R¹⁴ is independently selected from the group consisting of —OR¹⁷, —N(R¹⁷)₂, —NHR¹⁷, —NR²⁰R¹⁹ and —SR¹⁷;

R¹⁵ is selected from the group consisting of —H, lower alkyl, lower aryl and lower aralkyl, or together with R¹⁶ is connected via 2-6 atoms, optionally including 1 heteroatom selected from the group consisting of O, N, and S;

R¹⁶ is selected from the group consisting of —(CR¹²R¹³)ₙ—C(O)—R¹⁴, —H, lower alkyl, lower aryl and lower aralkyl, or together with R¹⁵ is connected via 2-6 atoms, optionally including 1 heteroatom selected from the group consisting of O, N, and S;

each R¹⁷ is independently selected from the group consisting of lower alkyl, lower aryl, and lower aralkyl, all optionally substituted, or together R¹⁷ and R¹⁷ on N is connected via 2-6 atoms, optionally including 1 heteroatom selected from the group consisting of O, N, and S;

R¹⁸ is independently selected from the group consisting of H, lower alkyl, aryl, and aralkyl, or together with R¹² is connected via 1-4 carbon atoms to form a cyclic group;

each R¹⁹ is independently selected from the group consisting of —H, lower alkyl, lower aryl, lower heterocycloalkyl, lower aralkyl, and COR³.

In one aspect, Y is independently selected from the group consisting of —O—, and —NR⁶—;

or when one Y—R¹ is —NR¹⁵(R¹⁶) then the other Y—R¹ is —N(R¹⁸)—(CR¹²R¹³)ₙ—C(O)—R¹⁴;

or when Y is —O—, then R¹ attached to —O— is independently selected from the group consisting of —H, —C(R²)₂—OC(O)R³, and —C(R²)₂—O—C(O)OR³,

or when Y is —NR⁶—, then R¹ attached to —NR⁶— is independently selected from the group consisting of —H, —[C(R²)₂]_{q}—COOR³, —C(R⁴)₂COOR³,

—[C(R²)₂]_{q}—C(O)SR, and -cycloalkylene-COOR³;

or when both Y's are —O—, then together R¹ and R¹ are 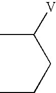
wherein

V is selected from the group consisting of optionally substituted monocyclic aryl and optionally substituted monocyclic heteroaryl.

In another aspect, both Y's are —O—, and together R¹ and R¹ are

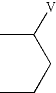

V is selected from the group consisting of phenyl, substituted phenyl with 1-3 substituents independently selected from the group consisting of —Cl, —Br, —F, C₁-C₃ alkyl, —CF₃, —COCH₃, —OMe, —NMe₂, —OEt, —CO₂t-butyl, and —CN, monocyclic heteroaryl, and substituted monocyclic heteroaryl with 1-2 substituents independently selected from the group consisting of —Cl, —Br, —F, C₁-C₃ alkyl, —CF₃, —COCH₃, —OMe, —NMe₂, —OEt, —CO₂t-butyl, and —CN and wherein said monocyclic heteroaryl and substituted monocyclic heteroaryl has 1-2 heteroatoms that are independently selected from the group consisting of N, O, and S with the provisos that

a) when there are two heteroatoms and one is O, then the other can not be O or S, and

b) when there are two heteroatoms and one is S, then the other can not be O or S.

In a further aspect, both Y groups are —O—. In another aspect, one Y is —NR⁶—, and one Y is —O—.

In yet another aspect, when Y is O, R¹ is independently selected from the group consisting of optionally substituted aryl, optionally substituted benzyl, —C(R²)₂OC(O)R³, —C(R²)₂OC(O)OR³, and —H; and

when Y is —NR⁶—, then the R¹ attached to said —NR⁶— group is selected from the group consisting of —C(R⁴)₂—COOR³, and —C(R²)₂COOR³; and the other Y group is —O— and then R¹ attached to said —O— is selected from the group consisting of optionally substituted aryl, —C(R²)₂OC(O)R³, and —C(R²)₂OC(O)OR³.

In another aspect, Y is O and R¹ is H. In a further aspect, one Y is —O—, and R¹ is optionally substituted aryl; and the other Y is —NR⁶—, where R¹ attached to said —NR⁶— is selected from the group consisting of —C(R⁴)₂COOR³ and —C(R²)₂C(O)OR³. In yet a further aspect, one Y—R¹ is —NR¹⁵(R¹⁶) and the other Y—R¹ is —N(R¹⁸)—(CR¹²R¹³)ₙ—C(O)—R¹⁴. In another aspect, both Y—R¹'s are —N(R¹⁸)—(CR¹²R¹³)ₙ—C(O)—R¹⁴.

In one aspect, both Y's are —O—, and together R¹ and R¹ are

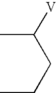

V is selected from the group consisting of phenyl, 3-chlorophenyl, 3-bromophenyl, 2-bromophenyl, 3,5-dichlorophenyl, 3-bromo-4-fluorophenyl, 2-pyridyl, 3-pyridyl, and 4-pyridyl.

In another aspect, both Y—R¹'s are —N(R¹⁸)—(CR¹²R¹³)ₙ—C(O)—R¹⁴, wherein n is 1, R¹⁸ is H, and R¹⁴ is —OR³. In a further aspect, R¹² is H; R¹³ is methyl; and the carbon bearing R¹² and R¹³ is in the (S)-configuration. In another aspect, R¹² is methyl and R¹³ is methyl.

In another aspect, when one Y is —NR¹⁵(R¹⁶) then the other Y is —N(R¹⁸)—(CR¹²R¹³)ₙ—C(O)—R¹⁴.

In one aspect, at least one R¹ is selected from the group consisting of —C(R²)₂—OC(O)R³ and —C(R²)₂—OC(O)OR³. In another aspect, R¹ attached to —O— is selected from the group consisting of phenyl and phenyl substituted with 1-2 substituents selected from the group consisting of —NHC(O)CH₃, —F, —Cl, —Br, —C(O)OCH₂CH₃, and —CH₃; and wherein R¹ attached to —NR⁶— is —C(R²)₂COOR³; and each R² is independently selected from the group consisting of —CH₃, —CH₂CH₃, and —H. In yet another aspect, R¹ attached to —O— is selected from the group consisting of phenyl and phenyl substituted with 1-2 substituents selected from the group consisting of 4-NHC(O)CH₃, —Cl, —Br, 2-C(O)OCH₂CH₃, and —CH₃.

In one aspect, R¹¹ is C₃-C₁₀ alkyl. In another aspect, R¹¹ is selected from the group consisting of methyl, ethyl, isopropyl, cyclobutyl, 3-pentyl and tert-butyl. In a further aspect, R¹¹ is selected from the group consisting of tert-butyl, 2-methyl-2-butyl, 3-methyl-3-pentyl, and 3-ethyl-3-pentyl. In yet another aspect, R¹¹ is tert-butyl. In another aspect, R¹¹ is isopropyl. In a further aspect, R¹¹ is 2-methyl-2-butyl.

In one aspect, R¹¹ is selected from the group consisting of methyl, ethyl, isopropyl, and tert-butyl; wherein when Y is —O—, then R¹ attached to —O— is independently selected from the group consisting of —H, optionally substituted phenyl, —CH₂OC(O)-tBu, —CH₂OC(O)Et, and —CH₂OC(O)-iPr;

when Y is —NR⁶—, then R¹ is attached to —NR⁶— independently selected from the group consisting of —C(R²)₂COOR³ and —C(R⁴)₂COOR³, or

when one Y—R¹ is —NR¹⁵(R¹⁶) then the other Y—R¹ is —N(R¹⁸)—(CR¹²R¹³)ₙ—C(O)—R¹⁴;

when Y is —O— or —NR⁶—, and at least one Y is —O—, then together R¹ and R¹ are

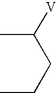

wherein

V is selected from the group consisting of optionally substituted aryl and optionally substituted heteroaryl;

R⁶ is selected from the group consisting of —H and lower alkyl.

In another aspect, R¹¹ is selected from the group consisting of methyl, ethyl, isopropyl, and tert-butyl; wherein when Y is —O—, then R¹ attached to —O— is independently selected from the group consisting of —H, —CH₂OC(O)-tBu, —CH₂OC(O)Et, and —CH₂OC(O)-iPr; when Y is —NR⁶—, then R¹ is attached to —NR⁶— independently selected from the group consisting of —C(R²)₂COOR³ and —C(R⁴)₂COOR³; and R⁶ is —H.

In a further aspect, R¹¹ is selected from the group consisting of methyl, ethyl, isopropyl, and tert-butyl; wherein when Y is —O—, then R¹ attached to —O— is —H; when Y is —NR⁶—, then R¹ attached to —NR⁶— is —C(R²)₂COOR³; and R⁶ is —H.

In yet another aspect, R¹¹ is selected from the group consisting of methyl, ethyl, isopropyl, and tert-butyl; wherein when Y is —O—, then R¹ attached to —O— is —H; when Y is —NR⁶—, then R¹ attached to —NR⁶— is —C(R²)₂COOR³; R² is H or methyl; R³ is ethyl or isopropyl; and R⁶ is —H.

In another aspect, R¹¹ is selected from the group consisting of methyl, ethyl, isopropyl, and tert-butyl; wherein each YR¹ is —OH. In a further aspect, R¹¹ is selected from the group consisting of methyl, ethyl, isopropyl, and tert-butyl; wherein each YR¹ is —NHC(Me)₂COOEt.

In a further aspect, R¹¹ is tert-butyl; wherein when Y is —O—, then R¹ attached to —O— is independently selected from the group consisting of —H, optionally substituted phenyl, —CH₂OC(O)-tBu, —CH₂OC(O)Et, and —CH₂OC(O)-iPr;

when Y is —NR⁶—, then R¹ is attached to —NR⁶— independently selected from the group consisting of —C(R²)₂COOR³ and —C(R⁴)₂COOR³, or

when one Y—R¹ is —NR¹⁵(R¹⁶) then the other Y—R¹ is —N(R¹⁸)—(CR¹²R¹³)ₙ—C(O)—R¹⁴;

when Y is —O— or —NR⁶—, and at least one Y is —O—, then together R¹ and R¹ are

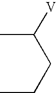

wherein

V is selected from the group consisting of optionally substituted aryl and optionally substituted heteroaryl;

R⁶ is selected from the group consisting of —H and lower alkyl.

In yet a further aspect, R¹¹ is tert-butyl; wherein when Y is —O—, then R¹ attached to —O— is independently selected from the group consisting of —H, —CH₂OC(O)-tBu, —CH₂OC(O)Et, and —CH₂OC(O)-iPr; when Y is —NR⁶—, then R¹ is attached to —NR⁶— independently selected from the group consisting of —C(R²)₂COOR³ and —C(R⁴)₂COOR³; and R⁶ is —H. In another aspect, R¹¹ is tert-butyl; wherein when Y is —O—, then R¹ attached to —O— is —H; when Y is —NR⁶—, then R¹ attached to —NR⁶— is —C(R²)₂COOR³; and R⁶ is —H. In a further aspect, R¹¹ is tert-butyl; wherein when Y is —O—, then R¹ attached to —O— is —H; when Y is —NR⁶—, then R¹ attached to —NR⁶— is —C(R²)₂COOR³; R² is H or methyl; R³ is ethyl or isopropyl; and R⁶ is —H. In one aspect, R¹¹ is tert-butyl and each YR¹ is —OH. In another aspect, R¹¹ is tert-butyl and each YR¹ is —NHC(Me)₂COOEt. In a further aspect, R¹¹ is tert-butyl and each YR¹ is —NHCH(Me)COOEt.

In one aspect, R¹¹ is isopropyl and each YR¹ is —OH. In another aspect, R¹¹ is isopropyl and each YR¹ is —NHC(Me)₂COOEt. In a further aspect, R¹¹ is isopropyl and each YR¹ is —NHCH(Me)COOEt. In a further aspect, R¹¹ is isopropyl; wherein when Y is —O—, then R¹ attached to —O— is independently selected from the group consisting of —H, optionally substituted phenyl, —CH₂OC(O)-tBu, —CH₂OC(O)Et, and —CH₂OC(O)-iPr;

when Y is —NR⁶—, then R¹ is attached to —NR⁶— independently selected from the group consisting of —C(R²)₂COOR³ and —C(R⁴)₂COOR³, or

when one Y—R¹ is —NR¹⁵(R¹⁶) then the other Y—R¹ is —N(R¹⁸)—(CR¹²R¹³)ₙ—C(O)—R¹⁴;

when Y is —O— or —NR⁶—, and at least one Y is —O—, then together R¹ and R¹ are

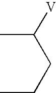

wherein

V is selected from the group consisting of optionally substituted aryl and optionally substituted heteroaryl;

R⁶ is selected from the group consisting of —H and lower alkyl.

In yet a further aspect, R¹¹ is isopropyl; wherein when Y is —O—, then R¹ attached to —O— is independently selected from the group consisting of —H, —CH₂OC(O)-tBu, —CH₂OC(O)Et, and —CH₂OC(O)-iPr; when Y is —NR⁶—, then R¹ is attached to —NR⁶— independently selected from the group consisting of —C(R²)₂COOR³ and —C(R⁴)₂COOR³; and R⁶ is —H. In another aspect, R¹¹ is isopropyl; wherein when Y is —O—, then R¹ attached to —O— is —H; when Y is —NR⁶—, then R¹ attached to —NR⁶— is —C(R²)₂COOR³; and R⁶ is —H. In a further aspect, R¹¹ is isopropyl; wherein when Y is —O—, then R¹ attached to —O— is —H; when Y is —NR⁶—, then R¹ attached to —NR⁶— is —C(R²)₂COOR³; R² is H or methyl; R³ is ethyl or isopropyl; and R⁶ is —H.

In one aspect, R¹¹ is 2-methyl-2-butyl and each YR¹ is —OH. In another aspect, R¹¹ is 2-methyl-2-butyl and each YR¹ is —NHC(Me)₂COOEt. In a further aspect, R¹¹ is 2-methyl-2-butyl and each YR¹ is —NHCH(Me)COOEt. In a further aspect, R¹¹ is 2-methyl-2-butyl; wherein when Y is —O—, then R¹ attached to —O— is independently selected from the group consisting of —H, optionally substituted phenyl, —CH₂OC(O)-tBu, —CH₂OC(O)Et, and —CH₂OC(O)-iPr;

when Y is —NR⁶—, then R¹ is attached to —NR⁶— independently selected from the group consisting of —C(R²)₂COOR³ and —C(R⁴)₂COOR³, or

when one Y—R¹ is —NR¹⁵(R¹⁶) then the other Y—R¹ is —N(R¹⁸)—(CR¹²R¹³)ₙ—C(O)—R¹⁴;

when Y is —O— or —NR⁶—, and at least one Y is —O—, then together R¹ and R¹ are

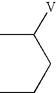

wherein

V is selected from the group consisting of optionally substituted aryl and optionally substituted heteroaryl;

R⁶ is selected from the group consisting of —H and lower alkyl.

In yet a further aspect, R¹¹ is 2-methyl-2-butyl; wherein when Y is —O—, then R¹ attached to —O— is independently selected from the group consisting of —H, —CH₂OC(O)-tBu, —CH₂OC(O)Et, and —CH₂OC(O)-iPr; when Y is —NR⁶—, then R¹ is attached to —NR⁶— independently selected from the group consisting of —C(R²)₂COOR³ and —C(R⁴)₂COOR³; and R⁶ is —H. In another aspect, R¹¹ is 2-methyl-2-butyl; wherein when Y is —O—, then R¹ attached to —O— is —H; when Y is —NR⁶—, then R¹ attached to —NR⁶— is —C(R²)₂COOR³; and R⁶ is —H. In a further aspect, R¹¹ is 2-methyl-2-butyl; wherein when Y is —O—, then R¹ attached to —O— is —H; when Y is —NR⁶—, then R¹ attached to —NR⁶— is —C(R²)₂COOR³; R² is H or methyl; R³ is ethyl or isopropyl; and R⁶ is —H.

Useful values for R¹¹ also include cycloalkyl such as cyclobutyl, cyclopentyl and cyclohexyl, thienyl, such as 2-thienyl, halophenyl, such as 3-fluorophenyl, 4-chlorophenyl, 3-chlorophenyl, 2-chlorophenyl and 4-fluorophenyl, alkylphenyl such as 4-methylphenyl, 3-methylphenyl and 2-methylphenyl, alkoxyphenyl such as 2-methoxyphenyl, 3-methoxyphenyl, 4-methoxyphenyl and 3,4-dimethoxyphenyl, 3,4-methylenedioxyphenyl, pyridyl such as 3-pyridyl, 3-chloro-4-(1-pyrrolidinyl)phenyl, 3-chloro-4-(1-morpholinyl)phenyl, 4-trifluoromethylphenyl, 3-trifluoromethylphenyl, 2-trifluoromethylphenyl, 4-phenylphenyl, naphthyl such as 2-naphthyl, piperidinyl such as 4-piperidinyl, and N,N-dimethylaminophenyl such as 4-(N,N-dimethylamino)phenyl.

In one aspect, the invention comprises a compound of the following formula:

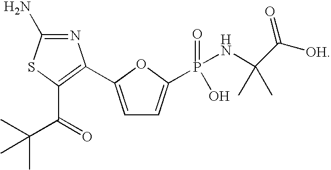

In one aspect, the salt form of a compound of Formula I is selected from the group consisting of methanesulfonate, ethanesulfonate, sulfate, hydrochloride, hydrobromide, acetate, citrate and tartrate.

Prodrugs of the 5-keto compounds of Formula I include compounds of the formula 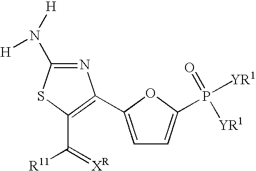
wherein X^{R} is ═S, ═S═O, ═N—R³ or ═N—OR², wherein R² and R³ and R¹ and R¹¹ are defined as above.

N-acetyltransferase (EC 2.3.1.5; NAT) is a Phase II drug-metabolizing enzyme that catalyzes the conjugation of an acetyl group from acetyl-CoA onto an amine, hydrazine or hydroxylamine moiety of an aromatic compound (reviewed in Upton A, Johnson N. Sandy J, Sim E, 2001, Trends Pharma. Sci. 22: 140-146). There are two NAT isozymes in humans, NAT1 and NAT2. The enzymes are polymorphic and have an important place in the history of pharmacogenetics, being first identified as responsible for the polymorphic inactivation of the anti-tubercular drug isoniazid. The genes expressing NAT1 and NAT2 are both located on chromosome 8 and share 87% and 81% nucleotide and amino acid sequence identity, respectively. NAT1 preferentially metabolizes p-aminobenzoate and p-aminosalicylate. Several allelic variants of NAT1 are known. Point mutations in the coding region of NAT1 generally result in reduced enzyme activity. The effect of mutations outside the coding region are controversial with one report indicating elevated activity and two others indicating similar activity. At least 15 different allelic variants of NAT2 have been identified to date, and their frequency in the population provides a molecular explanation for the polymorphic metabolism of model substrates such as sulfamethazine and procainamide. The representation of these mutant alleles differs widely between populations of different ethnic or geographical location, with slow acetylators accounting for 10% and 40-70% of Oriental and Caucasian populations, respectively. NAT2 is more active with heterocyclic amines as substrates than is NAT1. NAT2 is expressed in liver and intestinal epithelium, traditional sites of drug metabolism, whereas NAT1 is more ubiquitously expressed and predominates even in intestinal epithelium (Windmill K F, Gaedigk A, Hall P, et al, 2000, Tox. Sci. 54: 19-29).

N-acetylase activity can markedly influence the clinical pharmacokinetics of drugs. Susceptible drugs administered orally may be acetylated during passage through the intestinal epithelium thus reducing oral bioavailability. Any drug that gains entry to the circulation intact is then subject to further NAT metabolism in the liver or other target tissues, thus further reducing drug exposure. The degree to which drug exposure is altered is expected to exhibit significant interindividual variability as a result of the high frequency of rapid acetylator and slow acetylator phenotypes in the human population. Variable drug exposure and/or the formation of N-acetylated metabolites leads to altered efficacy and tolerability profiles for certain drugs. In patients treated with the antirheumatic drug (and NAT2 substrate) sulfasalazine, for instance, a correlation was found between efficacy and NAT2 genotype/phenotype; significantly shorter drug therapy was required in slow acetylators than in rapid acetylators (Kumagai S, Komada F, Kita T et al, 2004, Pharma. Res. 21:324-329). The ratio of active sulfasalazine metabolite to inactive/N-acetylated metabolite in plasma also correlated with NAT2 activity, with a higher ratio found in slow acetylators. Similarly, in the case of the antitubercular drug isoniazid, pronounced interindividual variation in circulating isoniazid concentration and clearance were observed clinically and these correlated with hereditary differences in acetylator status (Weber W W, Hein D W, Clin. Pharmacokinet. 4:401-422 (1979)). Slow acetylation of isoniazid decreased drug clearance and was linked to an increased risk of certain side effects (e.g. peripheral neuropathy) while efficacy appeared to be largely unaffected. The formation of toxic metabolites via N-acetylation can also be an issue. Batracylin, a heterocyclic amine with antitumor activity, is N-acetylated by NAT2. The acetyl product formed has been implicated in the toxicity of this agent in animals, cells and bacteria (Stevens G J, Payton M, Sim E, McQueen C A, Drug Metab. Dispos. 27:966-971 (1999)).

The structures of the compounds referred to in the description and examples below may be gleaned from the following table.

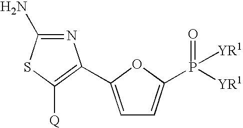

| Compound # | Q | YR¹ |
|---|---|---|
| 1.1 | 2,2-dimethylpropionyl | —OH |
| 1.2 | 2,2-dimethylbutyryl | —OH |
| 1.3 | 2-ethyl-2-methylbutyryl | —OH |
| 1.4 | Acetyl | —OH |
| 1.5 | Benzoyl | —OH |
| 1.6 | cyclohexylcarbonyl | —OH |
| 1.7 | 2-thienylcarbonyl | —OH |
| 1.8 | 3-fluorobenzoyl | —OH |
| 1.9 | 4-chlorobenzoyl | —OH |
| 1.10 | 4-methylbenzoyl | —OH |
| 1.11 | 3-methylbenzoyl | —OH |
| 1.12 | 3-chlorobenzoyl | —OH |
| 1.13 | 2-methylbenzoyl | —OH |
| 1.14 | 2-methoxybenzoyl | —OH |
| 1.15 | 2-chlorobenzoyl | —OH |
| 1.16 | 4-methoxybenzoyl | —OH |
| 1.17 | 3,4-dimethoxybenzoyl | —OH |
| 1.18 | 3-methoxybenzoyl | —OH |
| 1.19 | 3,4-methylenedioxybenzoyl | —OH |
| 1.20 | 3-pyridylcarbonyl | —OH |
| 1.21 | 3-chloro-4-(1- | —OH |
| | pyrrolidinyl) | |
| | benzoyl | |
| 1.22 | 4-fluorobenzoyl | —OH |
| 1.23 | 2-ethylbutyryl | —OH |
| 1.24 | 4-trifluoromethylbenzoyl | —OH |
| 1.25 | 3-chloro-4-(1- | —OH |
| | morpholinyl) | |
| | benzoyl | |
| 1.26 | 3-trifluoromethylbenzoyl | —OH |
| 1.27 | 2-trifluoromethylbenzoyl | —OH |
| 1.28 | 4-phenylbenzoyl | —OH |
| 1.29 | 2-naphthylcarbonyl | —OH |
| 1.30 | cyclopentylcarbonyl | —OH |
| 1.31 | 4-piperidinylbenzoyl | —OH |
| 1.32 | 4-(N,N-dimethylamino)- | —OH |
| | benzoyl | |
| 1.33 | 2-methylbutyryl | —OH |
| 1.34 | cyclobutylcarbonyl | —OH |
| 2.1 | 2,2-dimethylpropionyl | —NHC(Me)₂CO₂Et |
| 2.2 | 2,2-dimethylpropionyl | —NHCH(Me)CO₂Et (S) |
| 2.3 | 2,2-dimethylpropionyl | —NHC(Me)₂CO₂i-Pr |
| 2.4 | 2,2-dimethylpropionyl | —NHCH₂CO₂Et |
| 2.5 | 2-ethyl-2-methylbutyryl | —NHC(Me)₂CO₂Et |
| 2.6 | 2-ethyl-2-methylbutyryl | —NHC(Me)₂CO₂i-Pr |
| 2.7 | 2-ethyl-2-methylbutyryl | —NHCH(Me)CO₂Et (S) |
| 2.8 | 2,2-dimethylbutyryl | —NHC(Me)₂CO₂i-Pr |
| 2.9 | 2,2-dimethylbutyryl | —NHC(Me)₂CO₂Et |
| 2.10 | 2,2-dimethylbutyryl | —NHCH(Me)CO₂Et (S) |
| 2.11 | 2-ethyl-2-methylbutyryl | —NHCH(Me)CO₂i-Pr (S) |
| 2.12 | 2,2-dimethylbutyryl | —NHCH₂CO₂t-Bu |
| 2.13 | 2,2-dimethylpropionyl | —NHCH(Me)CO₂i-Pr (S) |
| 2.14 | 2-methylbenzoyl | —NHCH(Me)CO₂Et (S) |
| 2.15 | 2-methylbenzoyl | —NHC(Me)₂CO₂Et |
| 2.16 | 4-methylbenzoyl | —NHCH(Me)CO₂Et (S) |
| 2.17 | 4-methylbenzoyl | —NHC(Me)₂CO₂Et |
| 2.18 | 3-fluorobenzoyl | —NHCH(Me)CO₂i-Pr (S) |
| 2.19 | 3-fluorobenzoyl | —NHCH(Me)CO₂Et (S) |
| 2.20 | 4-methylbenzoyl | —NHC(Me)₂CO₂i-Pr |
| 2.21 | 2-methylbenzoyl | —NHC(Me)₂CO₂i-Pr |
| 2.22 | 2-methylbenzoyl | —NHCH(Me)CO₂i-Pr (S) |
| 2.23 | 2-ethylbutyryl | —NHC(Me)₂CO₂Et |
| 2.24 | 2-ethylbutyryl | —NHCH(Me)CO₂Et (S) |
| 2.25 | 2-ethylbutyryl | —NHC(Me)₂CO₂i-Pr |
| 2.26 | 2-ethylbutyryl | —NHCH(Me)CO₂i-Pr (S) |
| 2.27 | 3-fluorobenzoyl | —NHC(Me)₂CO₂Et |
| 2.28 | 3-fluorobenzoyl | —NHC(Me)₂CO₂i-Pr |
| 2.29 | cyclobutylcarbonyl | —NHC(Me)₂CO₂Et |
| 2.30 | cyclobutylcarbonyl | —NHCH(Me)CO₂Et (S) |
| 2.31 | cyclobutylcarbonyl | —NHCH(Me)CO₂i-Pr (S) |
| 2.32 | cyclobutylcarbonyl | —NHC(Me)₂CO₂i-Pr |
| 3.1 | 2,2-dimethylpropyl | —OH |
| 3.2 | cyclopentylmethyl | —OH |
| 3.3 | 2,2-dimethylbutyl | —OH |
| 3.4 | 2-propyl | —OH |
| 3.5 | 2-methylbutyl | —OH |
| 3.6 | 2-methylpropyl | —OH |
| 4.1 | 2,2-dimethylpropyl | —NHCH(Me)CO₂Et (S) |
| 4.2 | Phenyl | —NHCH(Me)CO₂Et (S) |
| 4.3 | Cyclohexyl | —NHCH(Me)CO₂i-Pr (S) |
| 4.4 | 2,2-dimethylpropyl | —NHCH₂CO₂Et |
| 4.5 | 2,2,3-trimethylbutyl | —NHCH(Me)CO₂Et (S) |
| 4.6 | 2-methylpropyl | —NHCH(Me)CO₂Et (S) |

As illustrated in Example C, FBPase inhibitors of the 2-amino-thiazole class with C-5 alkyl substitutions (e.g. 3.1, 3.6) were found to be highly susceptible to N-acetylation by human recombinant NAT1, and, where tested, to a lesser extent by NAT2. In addition, prodrugs of these inhibitors (e.g. 4.1, 4.6, see published international patent application WO 01/47935 A2, also published as U.S. patent application publication no. 2002/0173490 A1, incorporated herein by reference in its entirety) were extensively metabolized by human recombinant NAT2 and to a lesser extent by NAT1. These results are in agreement with the known SAR of NAT1, which metabolizes charged substrates, and that of NAT2, which has a preference for uncharged substrates.

Exploration of the SAR of substitutions at the C-5 position resulted in the discovery of a series of potent C5-keto-thiazole inhibitors (e.g. 1.1, 1.2, 1.3; Example A) which surprisingly were not metabolized by NAT1 (or NAT2, where tested). Moreover, the prodrugs of keto-thiazole inhibitors prepared (e.g. 2.1, 2.2, 2.3) were found to be insusceptible to N-acetylation by NAT2 (or NAT1 where tested). Prodrug 2.1 activated readily in liver S9 fractions ((Example D), showed good oral bioavailability (Examples H, I, and L), potent glucose lowering in normal rats (Example G), and sustained, dose-responsive glucose lowering in diabetic rats (Example J).

The insusceptibility of keto-thiazole inhibitors and their prodrugs to metabolism by NATs is expected to confer several key pharmacokinetic, therapeutic, and other advantages. NAT activity is highly expressed in the human intestine (Hickman D, Pope J, Patil S D et al., 1998, Gut 42: 402-409). Compounds that are susceptible to N-acetylation are extensively metabolized during passage across the intestinal wall into the general circulation. This reduces the oral bioavailability of the drug and consequently results in reduced potency. Compound 3.6 and its prodrug form, 4.6, are both susceptible to N-acetylation (Example C). Once acetylated, 4.6 may still be metabolically converted to N-acetyl-3.6. N-acetyl-3.6, however, is a very poor inhibitor of FBPase relative to 3.6 (Example A). The N-acetylation of either 3.6 or 4.6 thus results in drug inactivation. Compound 1.1 and its prodrug form, 2.1, in contrast to 3.6 and 4.6, are insusceptible to N-acetylation by either NAT1 or NAT2 (Example C). The insusceptibility of 1.1 and 2.1 to N-acetylation is likely an important factor in the 1.5-fold increased oral bioavailability of 2.1 relative to 4.6 (Examples H and I). Another important factor may be the decreased hydrophilicity of the 2-amino group that results from the presence of an electron-withdrawing keto group at the 5-position of the thiazole. This difference in oral bioavailability may be more pronounced in certain drug formulations which increase the intestinal transit time and thus the exposure of susceptible drugs to N-acetylase activity. The increased oral bioavailability of 2.1 translates to increased potency in type 2 diabetic patients. Compound 2.1 is consequently administered at a lower dose in patients. This is advantageous with respect to the cost of goods for the manufacturer. The lower dose also translates to a reduced risk of non-specific side effects which may be associated with the administration of FBPase inhibitors at higher doses.

The liver is another key human tissue in which high NAT activity is present (Jenne J W, 1965, J. Clin. Invest. 44: 1992-2002). Following oral administration in prodrug form, FBPase inhibitors distribute at high levels to the liver in vivo (Example E) and exert their pharmacological action (glucose lowering) by inhibiting the pathway of gluconeogenesis in this organ. Susceptibility to NAT results in reduced exposure and a reduced half-life of the active inhibitor. The latter results in a loss of potency and a reduced pharmacodynamic half-life. As illustrated in Example J, the pharmacodynamic half life of 1.1 following administration of 2.1 to the ZDF rat (duration of action>9 h) is significantly longer than that of the N-acetylation-susceptible 3.6 administered in 4.6 prodrug form (duration of action˜3 h).

FBPase inhibitors and their prodrugs that are susceptible to N-acetylation are administered multiple times per day in type 2 diabetics due to lower oral bioavailability and reduced pharmacodynamic half-life. Keto-thiazole FBPase inhibitors and their prodrugs (e.g. 2.1) that are N-acetylation resistant and demonstrate higher oral bioavailability and a longer pharmacodynamic half-life are administered once or at most twice a day in patients. The ease of use and thus the degree of patient compliance is significantly improved for prodrugs of keto-thiazole FBPase inhibitors as a result of the simplified dosing regimen.

N-acetylase activity is highly variable in humans due to genetic polymorphisms; it differs widely between populations of different ethnic or geographical locations (Grant D M, Hughes N C, Janezic S A et al., 1997, Mutat Res. 376: 61-70). Allelic variants of NAT1 are known that reduce enzyme activity (Lin H G, 1998, Pharmacogenetics 8: 269-281), whereas the phenotypes resulting from the hereditary polymorphism of NAT 2 can be divided into slow acetylators, intermediate acetylators, and rapid acetylators (Evans D A P, 1989, Pharmacol. Ther. 42: 157-234). The high variability in N-acetylase activity is readily apparent in a recent survey conducted by Gentest (Woburn, Mass.). The survey comprised an assessment of enzyme activity in liver cytosol obtained from 22 human donors (male and female Caucasian, African American. Asian, and Hispanic subjects). NAT1 activity, assayed using the standard substrate p-aminosalicyclic acid, ranged from 5.8 to 1300 nmoles product/mg protein/min. (average ±SD 176±274). NAT2 activity, assayed using the standard substrate sulfamethazine, ranged from 21-360 nmoles product/mg protein/min. (average ±SD 140±119). Individual values for liver N-acetylase activity are shown in the table below.

| | | NAT1 | |
|---|---|---|---|
| Catalog no.* | Donor profile | activity** | NAT2 activity** |
| 452801 | Female Caucasian | 24 | 340 |
| 452803 | Male Caucasian | 110 | 76 |
| 452806 | Female African American | 180 | 40 |
| 452818 | Male Caucasian | 240 | 170 |
| 452823 | Male Caucasian | 200 | Nd |
| 452827 | Female Caucasian | 24 | 210 |
| 452830 | Female Caucasian | 120 | 39 |
| 452831 | Male Caucasian | 8.2 | 240 |
| 452834 | Male Caucasian | 5.8 | 24 |
| 452835 | Male Caucasian | 290 | 27 |
| 452840 | Female Caucasian | 110 | 340 |
| 452842 | Male Asian | 470 | 200 |
| 452843 | Female Caucasian | 10 | 38 |
| 452847 | Female Caucasian | 1300 | 23 |
| 452856 | Female African American | 89 | 21 |
| 452864 | Female Caucasian | 140 | 360 |
| 452866 | Male Caucasian | 56 | 37 |
| 452870 | Female Caucasian | 58 | 65 |
| 452889 | Female Caucasian | 210 | 210 |
| 452893 | Female Caucasian | 130 | 210 |
| 452895 | Female Hispanic | 44 | 240 |
| 4528112 | Female Caucasian | 58 | 38 |

*Data obtained from the online catalog of Gentest (Woburn, MA; website www.gentest.com)
**Activity is expressed as nmoles product formed/mg of protein/min.

The differences in enzyme activity illustrated above can lead to high inter-individual variability in the metabolism of N-acetylase-susceptible drugs (Example K) and this may influence pharmacokinetics (e.g. oral bioavailability, half-life, Cmax) as well as efficacy. FBPase inhibitors and their prodrugs that are susceptible to N-acetylation demonstrate a variable pharmacological response in type 2 diabetic patients. When these drugs are co-administered with other N-acetylase-susceptible drugs this inter-individual variability is exacerbated as each drug interferes with the metabolism and consequently the pharmacokinetics and pharmacological response of the other. Prodrugs of keto-thiazole FBPase inhibitors (e.g. 2.1) show a uniform pharmacological response in type 2 diabetic patients and a low non-responder rate. Furthermore, they have significantly less potential for drug-drug interactions when co-administered with N-acetylase susceptible drugs.

The formation of N-acetylated metabolites may adversely affect the safety profile of drugs. The metabolites may interact with receptors and/or enzymes thereby altering cellular metabolism/organ function and causing toxicity. In certain cases N-acetylation may lead to the formulation of carcinogenic metabolites (Hein D W, Cancer Epidemiol. Biomarkers Prev. 9:9-42 (2000)). The pharmacokinetics of N-acetylated metabolites is unpredictable; they may accumulate in certain tissues or the circulation due to their low renal or hepatic clearance. Accumulation exacerbates any safety issues associated with these metabolites. Keto-thiazole FBPase inhibitors (e.g. 1.1) and their prodrugs (e.g. 2.1) are not susceptible to N-acetylation. The propensity for safety issues relating to the formation and/or accumulation of N-acetylated metabolites is thus eliminated for drugs such as 2.1 when administered to type 2 diabetic patients.

Exploration of the amino acid moiety of the bisamidate prodrugs (wherein Y—R¹, is an amino acid and Y is NH) of compounds of Formula I revealed that 2-methylalanine as HY—R¹ displays distinct advantages with respect to oral bioavailability and efficiency of prodrug activation. The 2-methylalanine bisamidate prodrug of compound 1.1 showed 3-fold higher oral bioavailability compared to its corresponding L-alanine bisamidate prodrug (Example H: compounds 2.1 and 2.2, oral bioavailability of 30 and 11% respectively). Additionally, 2-methylalanine bisamidate prodrugs are often observed to be more efficiently converted into their corresponding active moieties. For example, assay of in vitro prodrug activation using liver S9 (Example D) showed that compound 2.1 (a 2-methylalanine bisamidate prodrug) was converted to its active moiety 1.6- to 4-fold more rapidly than compound 4.6 (an L-alanine bisamidate prodrug).

Formulations

Compounds of the invention are administered in a total daily dose of 0.01 to 2500 mg. In one aspect the range is about 5 mg to about 500 mg. The dose may be administered in as many divided doses as is convenient.

Compounds of this invention may be used in combination with other pharmaceutical agents. The compounds may be administered as a daily dose or an appropriate fraction of the daily dose (e.g., bid). Administration of the compound may occur at or near the time in which the other pharmaceutical agent is administered or at a different time. The compounds of this invention may be used in a multidrug regimen, also known as combination or ‘cocktail’ therapy, wherein, multiple agents may be administered together, may be administered separately at the same time or at different intervals, or administered sequentially. The compounds of this invention may be administered after a course of treatment by another agent, during a course of therapy with another agent, administered as part of a therapeutic regimen, or may be administered prior to therapy by another agent in a treatment program.

For the purposes of this invention, the compounds may be administered by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters. Intravenous administration is generally preferred.

Pharmaceutically acceptable salts include acetate, adipate, besylate, bromide, camsylate, chloride, citrate, edisylate, estolate, fumarate, gluceptate, gluconate, glucoranate, hippurate, hyclate, hydrobromide, hydrochloride, iodide, isethionate, lactate, lactobionate, maleate, mesylate, methylbromide, methylsulfate, napsylate, nitrate, oleate, palmoate, phosphate, polygalacturonate, stearate, succinate, sulfate, sulfosalicylate, tannate, tartrate, terphthalate, tosylate, and triethiodide.

Pharmaceutical compositions containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the invention contain the active materials in admixture with excipients suitable for the manufacture of aqueous suspensions. Such excipients include a suspending agent, such as sodium carboxymethylcellulose, methylcellulose, ethylcellulose, hydroxypropylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachid oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules of the invention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the invention may also be in the form of oil-in-water emulsions. The oily phase may be a vegetable oil, such as olive oil or arachid oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents.

Syrups and elixirs may be formulated with sweetening agents, such as glycerol, sorbitol or sucrose. Such formulations may also contain a demulcent, a preservative, a flavoring or a coloring agent.

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain 20 to 2000 μmol (approximately 10 to 1000 mg) of active material compounded with an appropriate and convenient amount of carrier material which may vary from about 5 to about 95% of the total compositions. It is preferred that the pharmaceutical composition be prepared which provides easily measurable amounts for administration. For example, an aqueous solution intended for intravenous infusion should contain from about 0.05 to about 50 μmol (approximately 0.025 to 25 mg) of the active ingredient per milliliter of solution in order that infusion of a suitable volume at a rate of about 30 mL/hr can occur.

As noted above, formulations of the present invention suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach. This is particularly advantageous with the compounds of Formula I when such compounds are susceptible to acid hydrolysis.

Formulations suitable for topical administration in the mouth include lozenges comprising the active ingredient in a flavored base, usually sucrose and acacia or tragacanth; pastilles comprising the active ingredient in an inert base such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous isotonic sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Injection solutions and suspensions may be prepared from sterile powders, granules and tablets of the kind previously described.

Formulations suitable for parenteral administration may be administered in a continuous infusion manner via an indwelling pump or via a hospital bag. Continuous infusion includes the infusion by an external pump. The infusions may be done through a Hickman or PICC or any other suitable means of administering a formulation either parenterally or i.v.

Preferred unit dosage formulations are those containing a daily dose or unit, daily sub-dose, or an appropriate fraction thereof, of a drug.

It will be understood, however, that the specific dose level for any particular patient will depend on a variety of factors including the activity of the specific compound employed; the age, body weight, general health, sex and diet of the individual being treated; the time and route of administration; the rate of excretion; other drugs which have previously been administered; and the severity of the particular disease undergoing therapy, as is well understood by those skilled in the art.

Synthesis of the Compounds of the Invention

The compounds in this invention may be prepared by the processes described in the following discussions, as well as relevant published literature procedures that are used by those skilled in the art. It should be understood that the following discussions are provided solely for the purpose of illustration and do not limit the invention which is defined by the claims. Typically the synthesis of a compound of Formula I includes the following general steps (listed in reversed order): (1) Preparation of a prodrug; (2) Deprotection of a phosphonate ester; (3) Modifications of an existing thiazole; (4) Construction of a thiazole; and (5) Preparation of key precursors. Protection and deprotection in the Schemes may be carried out according to the procedures generally known in the art (e.g., “Protecting Groups in Organic Synthesis,” 3rd Edition, Wiley, 1999).

All stereoisomers of the compounds of the instant invention are contemplated, either in admixture or in pure or substantially pure form. The compounds of the present invention can have stereogenic centers at the phosphorus atom and at any of the carbons including any of the R substituents. Consequently, compounds of Formula I can exist in enantiomeric or diastereomeric forms or in mixtures thereof. The processes for preparation can utilize racemates, enantiomers or diastereomers as starting materials. When enantiomeric or diastereomeric products are prepared, they can be separated by conventional methods. For example, chromatography or fractional crystallization can be used to separate diastereomeric mixtures, while derivatives of enantiomeric isomers can be separated via chromatography.

1) Preparation of a Prodrug

Prodrugs can be introduced at different stages of the synthesis. Most often these prodrugs are introduced at the later stage of a synthesis due to the lability of various prodrugs.

Phosphonic acids of Formula I wherein both Ys are O and R¹ is H, which may be in a suitably protected form, can be alkylated with electrophiles such as alkyl halides and alkyl sulfonates under nucleophilic substitution conditions to give phosphonate esters. For example, compounds of Formula I wherein Y is O and R¹ is an acyloxyalkyl group can be prepared by direct alkylation of compounds of Formula I wherein both Ys are O and R¹ is H with an appropriate acyloxyalkyl halide (e.g. Cl, Br, I; Phosphorus Sulfur 1990, 54, 143; Synthesis 1988, 62) in the presence of a suitable base (e.g. pyridine, TEA, diisopropylethylamine) in suitable solvents such as DMF (J. Med. Chem. 1994, 37, 1875). The carboxylate component of these acyloxyalkyl halides includes but is not limited to acetate, propionate, isobutyrate, pivalate, benzoate, carbonate and other carboxylates.

Dimethylformamide dialkyl acetals can also be used for the alkylation of phosphonic acids (Collect. Czech Chem. Commu. 1994, 59, 1853). Compounds of Formula I wherein Y is O and R¹ is a cyclic carbonate, a lactone or a phthalidyl group can also be synthesized by direct alkylation of the free phosphonic acids with appropriate halides in the presence of a suitable base such as NaH or diisopropylethylamine (J. Med. Chem. 1995, 38, 1372; J. Med. Chem. 1994, 37, 1857; J. Pharm. Sci. 1987, 76, 180).

Alternatively, these phosphonate prodrugs can be synthesized by the reactions of the corresponding dichlorophosphonates and an alcohol (Collect Czech Chem. Commun. 1994, 59, 1853). For example, a dichlorophosphonate is reacted with substituted phenols and arylalkyl alcohols in the presence of a base such as pyridine or TEA to give the compounds of Formula I wherein Y is O and R¹ is an aryl group (J. Med. Chem. 1996, 39, 4109; J. Med. Chem. 1995, 38, 1372; J. Med. Chem. 1994, 37, 498) or an arylalkyl group (J. Chem. Soc. Perkin Trans. 1 1992, 38, 2345). The disulfide-containing prodrugs (Antiviral Res. 1993, 22, 155) can be prepared from a dichlorophosphonate and 2-hydroxyethyldisulfide under standard conditions.

Dichlorophosphonates are also useful for the preparation of various phosphonamides as prodrugs. For example, treatment of a dichlorophosphonate with an amine (e.g. an amino acid alkyl ester such as L-alanine ethyl ester) in the presence of a suitable base (e.g. triethylamine, pyridine, etc.) gives the corresponding bisphosphonamide; treatment of a dichlorophosphonate with 1-amino-3-propanol gives a cyclic 1,3-propylphosphonamide; treatment of a chlorophosphonate monophenyl ester with an aminoacid ester in the presence of a suitable base gives a substituted monophenyl monophosphonamidate. Direct couplings of a phosphonic acid with an amine (e.g. an amino acid alkyl ester such as L-alanine ethyl ester) are also reported to give the corresponding bisamidates under Mukaiyama conditions (J. Am. Chem. Soc., 1972, 94, 8528).

Such reactive dichlorophosphonates can be generated from the corresponding phosphonic acids with a chlorinating agent (e.g. thionyl chloride, J. Med. Chem. 1994, 1857; oxalyl chloride, Tetrahedron Lett. 1990, 31, 3261; phosphorous pentachloride, Synthesis 1974, 490). Alternatively, a dichlorophosphonate can be generated from its corresponding disilyl phosphonate esters (Synth. Commu. 1987, 17, 1071) or dialkyl phosphonate esters (Tetrahedron Lett. 1983, 24, 4405; Bull. Soc. Chim. 1993, 130, 485).

It is envisioned that compounds of Formula I can be mixed phosphonate ester (e.g. phenyl and benzyl esters, or phenyl and acyloxyalkyl esters) including the chemically combined mixed esters such as phenyl and benzyl combined prodrugs reported in Bioorg. Med. Chem. Lett. 1997, 7, 99.

The SATE (S-acetyl thioethyl) prodrugs can be synthesized by the coupling reaction of the phosphonic acids of Formula I and S-acyl-2-thioethanol in the presence of DCC, EDCI or PyBOP (J. Med. Chem. 1996, 39, 1981).

Cyclic phosphonate esters of substituted 1,3-propane diols can be synthesized by either reactions of the corresponding dichlorophosphonate with a substituted 1,3-propanediol or coupling reactions using suitable coupling reagents (e.g. DCC, EDCI, PyBOP; Synthesis 1988, 62). The reactive dichlorophosphonate intermediates can be prepared from the corresponding acids and chlorinating agents such as thionyl chloride (J. Med. Chem., 1994, 1857), oxalyl chloride (Tetrahedron Lett., 1990, 31: 3261) and phosphorus pentachloride (Synthesis, 1974, 490). Alternatively, these dichlorophosphonates can also be generated from disilyl esters (Synth. Commun., 1987, 17: 1071) and dialkyl esters (Tetrahedron Lett., 1983, 24: 4405; Bull. Soc. Chim. Fr., 1993, 130: 485).

Alternatively, these cyclic phosphonate esters of substituted 1,3-propane diols are prepared from phosphonic acids by coupling with diols under Mitsunobu reaction conditions (Synthesis 1 (1981); J. Org. Chem. 52:6331 (1992)), and other acid coupling reagents including, but not limited to, carbodiimides (Collect. Czech. Chem. Commun. 59:1853 (1994); Bioorg. Med. Chem. Lett. 2:145 (1992); Tetrahedron Lett. 29:1189 (1988)), and benzotriazolyloxytris-(dimethylamino) phosphonium salts (Tetrahedron Lett. 34, 6743 (1993)).

Phosphonic acids also undergo cyclic prodrug formation with cyclic acetals or cyclic ortho esters of substituted propane-1,3-diols to provide prodrugs as in the case of carboxylic acid esters (Helv. Chim. Acta. 48:1746 (1965)). Alternatively, more reactive cyclic sulfites or sulfates are also suitable coupling precursors to react with phosphonic acid salts. These precursors can be made from the corresponding diols as described in the literature.

Alternatively, cyclic phosphonate esters of substituted 1,3-propane diols can be synthesized by trans esterification reaction with substituted 1,3-propane diol under suitable conditions. Mixed anhydrides of parent phosphonic acids generated in situ under appropriate conditions react with diols to give prodrugs as in the case of carboxylic acid esters (Bull. Chem. Soc. Jpn. 52:1989 (1979)). Aryl esters of phosphonates are also known to undergo transesterification with alkoxy intermediates (Tetrahedron Lett. 38:2597 (1997); Synthesis 968 (1993)).

A suitable prodrug of the 2-amino group of compounds of Formula I can also be prepared according reported procedures (J. Med. Chem., 47: 2393 (2004); Int. J. Antimicrob. Agents, 18: (451 (2001)).

In another aspect, a suitable prodrug of the 5-keto group of compounds of Formula I are also envisioned, which may lead to enhanced pharmaceutical properties such as solubility and stability etc. Therefore, prodrugs of the keto group at the C5-position of the thiazole ring in compounds of formula I can be prepared using conventional synthetic methods. For example, thioketones can be prepared from their corresponding ketones, and this transformation can be conducted either in the early stage of the synthesis or once the thiazole ring is already formed. Reagents suitable for such transformation include Lawesson's reagent under various conditions (Tetrahedron Lett., 42: 6167 (2001); J. Am. Chem. Soc., 125: 9560 (2003)). Furthermore, sulfoxides of thioketones can also be prepared from their corresponding thioketones under oxidative conditions using a suitable oxidant (e.g. mCPBA,); J. Am. Chem. Soc., 125: 12114 (2003)). Imines and oximes and their derivatives are also envisioned as potential prodrugs of the keto group at the C5-position of the thiazole ring for compounds of formula I. Imines and oximes are readily prepared from their corresponding ketones (Larock, Comprehensive organic transformations, VCH, New York, 1989). Moreover, various salt forms of imines and/or oximes can also be prepared such as methanesulfonic acid, hydrogen chloride salts.

One aspect of the present invention provides methods to synthesize and isolate single isomers of prodrugs of phosphonic acids of Formula I. Because phosphorus is a stereogenic atom, formation of a prodrug with a racemic substituted-1,3-propane-diol will produce a mixture of isomers. For example, formation of a prodrug with a racemic 1-(V)-substituted-1,3-propane diol gives a racemic mixture of cis-prodrugs and a racemic mixture of trans-prodrugs. In an other aspect, the use of the enantioenriched substituted-1,3-propane diol with the R-configuration gives enantioenriched R-cis- and R-trans-prodrugs. These compounds can be separated by a combination of column chromatography and/or fractional crystallization.

2) Deprotection of a Phosphonate Ester

Compounds of Formula I wherein R¹ is H may be prepared from phosphonate esters using known phosphate and phosphonate ester cleavage conditions. Silyl halides are generally used to cleave various phosphonate esters, and subsequent mild hydrolysis of the resulting silyl phosphonate esters give the desired phosphonic acids. When required, acid scavengers (e.g. 1,1,1,3,3,3-hexamethyldisilazane, 2,6-lutidine, etc.) can be used for the synthesis of acid labile compounds. Such silyl halides include chlorotrimethylsilane (Rabinowitz, J. Org. Chem., 1963, 28: 2975), and bromotrimethylsilane (McKenna, et al, Tetrahedron Lett., 1977, 155), and iodotrimethylsilane (Blackburn, et al., J. Chem. Soc., Chem. Commun., 1978, 870). Alternately, phosphonate esters can be cleaved under strong acidic conditions (e.g. HBr or HCl: Moffatt, et al, U.S. Pat. No. 3,524,846, 1970). These esters can also be cleaved via dichlorophosphonates, prepared by treating the esters with halogenating agents (e.g. phosphorus pentachloride, thionyl chloride, BBr₃: Pelchowicz et al., J. Chem. Soc., 1961, 238) followed by aqueous hydrolysis to give phosphonic acids. Aryl and benzyl phosphonate esters can be cleaved under hydrogenolysis conditions (Lejczak, et al., Synthesis, 1982, 412; Elliott, et al., J. Med. Chem., 1985, 28: 1208; Baddiley, et al., Nature, 1953, 171: 76) or metal reduction conditions (Shafer, et al., J. Am. Chem. Soc., 1977, 99: 5118). Electrochemical (Shono, et al., J. Org. Chem., 1979, 44: 4508) and pyrolysis (Gupta, et al., Synth. Commun., 1980, 10: 299) conditions have also been used to cleave various phosphonate esters.

(3) Modification of an Existing Thiazole

Although it is advantageous to have the desired substituents present when a thiazole ring is formed, in some cases, the desired substituents are not compatible with subsequent reactions, and therefore modifications of an existing thiazole are envisioned using conventional chemistry (Larock, Comprehensive organic transformations, VCH, New York, 1989; Trost, Comprehensive organic synthesis; Pergamon press, New York, 1991). For example, the 2-amino group of compounds of Formula I can be synthesized for the corresponding 2-bromothiazole analogs using transition metal catalyzed amination reactions. Alternatively, the 2-amino group can be obtained from the corresponding 2-carboxylic acid or its derivatives using conventional chemistry such as Curtius rearrangement and Beckman rearrangement reactions. Substitutions at the 4-position of thiazoles of Formula I can be introduced in various ways, if the desired group is not present when the thiazole is formed. For example, aryl groups are readily coupled onto a thiazole with a suitable C4-leaving group such as a bromo or triflate group using transition metal chemistry such as Stille and Suzuki reactions (Farina et al., Organic Reactions, Vol. 50; Wiley, New York, 1997; Mitchell, Synthesis, 1992, 808; Suzuki, Pure App. Chem., 1991, 63, 419). It is also possible that the keto group at the 5-position of compounds of Formula I may also be introduced once the thiazole is formed.

For example, the conventional acylation reactions (e.g. Friedel-Crafts reactions) can be used to introduce a keto group onto the 5-position of an unsubstituted thiazole; lithiation of a C5-unsubstituted thiazole followed by reaction with a suitable carbonyl derivative such as Weinreb's amide, or addition to an aldehyde followed by oxidation of the resulting alcohol will also give 5-ketothiazole analogs. Alternatively, transition metal chemistry can also be used to introduce a keto group to the 5-position of a thiazole. For example, a thiazole-5-stannyl derivative is reacted with a halide under carbon monoxide atmosphere to give 5-ketothiazole analogs, while couplings of organotin derivatives with acyl halides have often been reported to give ketone derivatives.

(4) Construction of a Thiazole

Aminothiazoles useful for the present invention can be readily prepared using well described ring-forming reactions (Metzger, Thiazole and its derivatives, part 1 and part 2; Wiley & Sons, New York, 1979). Cyclization reactions of thiourea and alpha-halocarbonyl compounds (such as alpha-haloketones, alpha-haloaldehydes) are particularly useful for the construction of an aminothiazole ring system. For example, cyclization reactions between thiourea and 5-diethylphosphono-2-[(2-bromo-1,3-dioxo)alkyl]furans are useful for the synthesis of compounds of Formula I wherein R¹¹ is an alkyl group. In this case, two aminothiazole regioisomers may be formed; acquisition of the desired regioisomer may be controlled by appropriate selection of conditions for both the cyclization reaction and isolation of the product.

Alpha-halocarbonyl compounds are readily accessible via conventional reactions (Larock, Comprehensive organic transformations, VCH, New York, 1989). Ketones can be halogenated using various halogenating reagents (e.g. NBS, CuBr₂, SO₂Cl₂); some examples are given in the following section.

(5) Preparation of Various Precursors Useful for Cyclization Reactions

A. Preparation of General Key Intermediates

Intermediates required for the synthesis of compounds in the present invention are generally prepared using either an existing method in the literature or a modification of an existing method. Syntheses of some of the intermediates useful for the synthesis of compounds in the present invention are described herein.

Various aryl phosphonate dialkyl esters are particularly useful for the synthesis of compounds of Formula I. For example, compounds of Formula I can be prepared from a variety of furanyl precursors. 5-Dialkylphosphono-2-furancarbonyl compounds (e.g. 5-diethylphosphono-2-furaldehyde, 5-diethylphosphono-furan-2-yl ketones) are well suited for the synthesis of compounds of Formula I. These intermediates are prepared from furan or furan derivatives using conventional chemistry such as lithiation reactions, protection of carbonyl groups and deprotection of carbonyl groups. For example, lithiation of furan using known methods (Gschwend Org. React. 1979, 26: 1) followed by addition of phosphorylating agents (e.g. ClPO₃R₂) gives 2-dialkylphosphono-furans (e.g. 2-diethylphosphonofuran). This method can also be applied to a 2-substituted furan such as 2-furoic acid to give a 5-dialkylphosphono-2-substituted furan such as 5-diethylphosphono-2-furoic acid. Alternatively, other methods such as transition metal catalyzed reactions of aryl halides or triflates (Balthazar et al., J. Org. Chem., 1980, 45: 5425; Petrakis et al., J. Am. Chem. Soc., 1987, 109: 2831; Lu et al., Synthesis, 1987, 726) are used to prepare aryl phosphonates.

A second lithiation step can be used to incorporate a second group on the furan-2-yl phosphonate dialkyl ester such as an aldehyde group, a trialkylstannyl, a keto group or a halo group, although other methods known to generate these functionalities (e.g. aldehydes) can be envisioned as well. For example, Vilsmeier-Haack reaction or Reimar-Teimann reactions can be used for aldehyde synthesis, while Friedel-Crafts reactions can be used to prepare keto-furan derivatives. In the second lithiation step, the lithiated furan ring is treated with reagents that either directly generate the desired functional group (e.g. for an aldehyde using DMF, HCO₂R, etc.) or with reagents that lead to a group that is subsequently transformed into the desired functional group using known chemistry (e.g. alcohols, esters, nitriles, alkenes can be transformed into aldehydes). For example, lithiation of a 2-dialkylphosphonofuran (e.g. 2-diethylphosphonofuran) under normal conditions (e.g. LDA in THF) followed by trapping of the thus generated anion with an electrophile (e.g. tributyltin chloride or iodine) produces a 5-functionalized-2-dialkylphosphonofuran (e.g. 5-tributylstannyl-2-diethylphosphonofuran or 5-iodo-2-diethylphosphonofuran). It is also envisioned that the sequence of these reactions can be reversed, i.e. the aldehyde moiety can be incorporated first followed by the phosphorylation reaction. The order of the reaction will be dependent on reaction conditions and protecting groups. Prior to the phosphorylation, it is also envisioned that it may be advantageous to protect some of these functional groups using a number of well-known methods (e.g. protection of aldehydes as acetals, aminals; protection of ketones as ketals). The protected functional group is then unmasked after phosphorylation. (Protective groups in Organic Synthesis, Greene, T. W., 1991, Wiley, New York). For example, protection of 2-furaldehyde as 1,3-propanediol acetal followed by a lithiation step (using for example LDA) and trapping the anion with a dialkyl chlorophosphate (e.g. diethyl chlorophosphate), and subsequent deprotection of the acetal functionality under normal deprotection conditions produces the 5-dialkylphosphono-2-furaldehyde (e.g. 5-diethylphosphono-2-furaldehyde). Another example is the preparation of 5-keto-2-dialkylphosphonofurans which encompass the following steps: acylations of furan under Friedel-Crafts reaction conditions give 2-ketofuran, subsequent protection of the ketone as ketals (e.g. 1,3-propanediol cyclic ketal) followed by a lithiation step as described above gives the 5-dialkylphosphono-2-furanketone with the ketone being protected as a 1,3-propanediol cyclic ketal, and final deprotection of the ketal under, for example, acidic conditions gives 2-keto-5-dialkylphosphonofurans (e.g. 2-acetyl-5-diethylphosphonofuran). Alternatively, 2-ketofurans can be synthesized via a palladium catalyzed reaction between 2-trialkylstannylfurans (e.g. 2-tributylstannylfuran) and an acyl chloride (e.g. acetyl chloride, isobutyryl chloride). It is advantageous to have the phosphonate moiety present in the 2-trialkylstannylfurans (e.g. 2-tributylstannyl-5-diethylphosphonofuran). 2-Keto-5-dialkylphosphonofurans can also be prepared from a 5-dialkylphosphono-2-furoic acid (e.g. 5-diethylphosphono-2-furoic acid) by conversion of the acid to the corresponding acyl chloride or a Weinreb's amide and followed by additions of a Grignard reagent.

Some of the above described intermediates can also be used for the synthesis of other useful intermediates. For example, a 2-keto-5-dialkylphosphonofuran can be further converted to a 1,3-dicarbonyl derivative such as a 5-(1,3-dioxo-alkyl)furan-2-yl phosphonate dialkyl ester, which is further converted to a 5-(2-halo-1,3-dioxo-alkyl)furan-2-yl phosphonate dialkyl ester that is useful for the reaction with a thioamide (e.g. thiourea) to give thiazole analogs.

It is conceivable that when applicable the above described synthetic methods can be adapted for parallel synthesis either on solid phase or in solution to provide rapid SAR (structure activity relationship) exploration of FBPase inhibitors encompassed in the current invention, provided method development for these reactions is successful.

B. Preparation of 1,3-Diols

Various methods can be used to prepare 1,3-propanediols such as 1-substituted, 2-substituted, 1,2- or 1,3-annulated 1,3-propanediols.

1. 1-Substituted 1,3-propanediols

1,3-Propanediols useful in the synthesis of compounds in the present invention can be prepared using various synthetic methods. As described in Scheme 10, additions of an aryl Grignard to a 1-hydroxy-propan-3-al give 1-aryl-substituted 1,3-propanediols (path a). This method is suitable for the conversion of various aryl halides to 1-arylsubstituted-1,3-propanediols (J. Org. Chem. 1988, 53, 911). Conversions of aryl halides to 1-substituted 1,3-propanediols can also be achieved using Heck reactions (e.g. couplings with a 1,3-diox-4-ene) followed by reductions and subsequent hydrolysis reactions (Tetrahedron Lett. 1992, 33, 6845). Various aromatic aldehydes can also be converted to 1-substituted-1,3-propanediols using alkenyl Grignard addition reactions followed by hydroboration-oxidation reactions (path b).

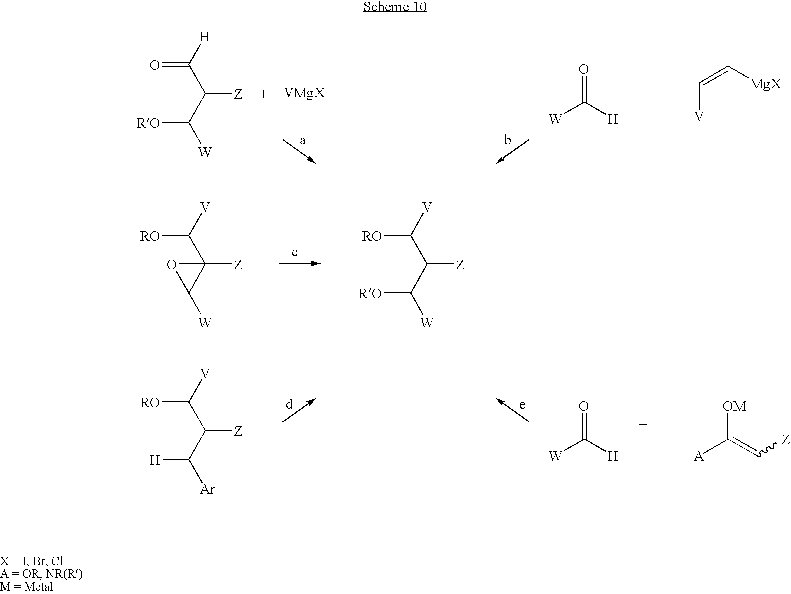

Aldol reactions between an enolate (e.g. lithium, boron, tin enolates) of a carboxylic acid derivative (e.g. tert-butyl acetate) and an aldehyde (e.g. the Evans's aldol reactions) are especially useful for the asymmetric synthesis of enantioenriched 1,3-propanediols. For example, reaction of a metal enolate of t-butyl acetate with an aromatic aldehyde followed by reduction of the ester (path e) gives a 1,3-propanediol (J. Org. Chem. 1990, 55 4744). Alternatively, epoxidation of cinnamyl alcohols using known methods (e.g. Sharpless epoxidations and other asymmetric epoxidation reactions) followed by reduction reactions (e.g. using Red-Al) give various 1,3-propanediols (path c). Enantioenriched 1,3-propanediols can be obtained via asymmetric reduction reactions (e.g. enantioselective borane reductions) of 3-hydroxy-ketones (Tetrahedron Lett. 1997, 38 761). Alternatively, resolution of racemic 1,3-propanediols using various methods (e.g. enzymatic or chemical methods) can also give enantioenriched 1,3-propanediol. Propan-3-ols with a 1-heteroaryl substituent (e.g. a pyridyl, a quinolinyl or an isoquinolinyl) can be oxygenated to give 1-substituted 1,3-propanediols using N-oxide formation reactions followed by a rearrangement reaction in acetic anhydride conditions (path d) (Tetrahedron 1981, 37, 1871).

2. 2-Substituted 1,3-propanediols

A variety of 2-substituted 1,3-propanediols useful for the synthesis of compounds of Formula I can be prepared from various other 1,3-propanediols (e.g. 2-(hydroxymethyl)-1,3-propanediols) using conventional chemistry (Comprehensive Organic Transformations, VCH, New York, 1989). For example, as described in Scheme 11, reductions of a trialkoxycarbonylmethane under known conditions give a triol via complete reduction (path a) or a bis(hydroxymethyl)acetic acid via selective hydrolysis of one of the ester groups followed by reduction of the remaining two other ester groups. Nitrotriols are also known to give triols via reductive elimination (path b) (Synthesis 1987, 8, 742). Furthermore, a 2-(hydroxymethyl)-1,3-propanediol can be converted to a mono acylated derivative (e.g. acetyl, methoxycarbonyl) using an acyl chloride or an alkyl chloroformate (e.g. acetyl chloride or methyl chloroformate) (path d) using known chemistry (Protective Groups In Organic Synthesis; Wiley, New York, 1990). Other functional group manipulations can also be used to prepare 1,3-propanediols such as oxidation of one the hydroxymethyl groups in a 2-(hydroxymethyl)-1,3-propanediol to an aldehyde followed by addition reactions with an aryl Grignard (path c). Aldehydes can also be converted to alkyl amines via reductive amination reactions (path e).

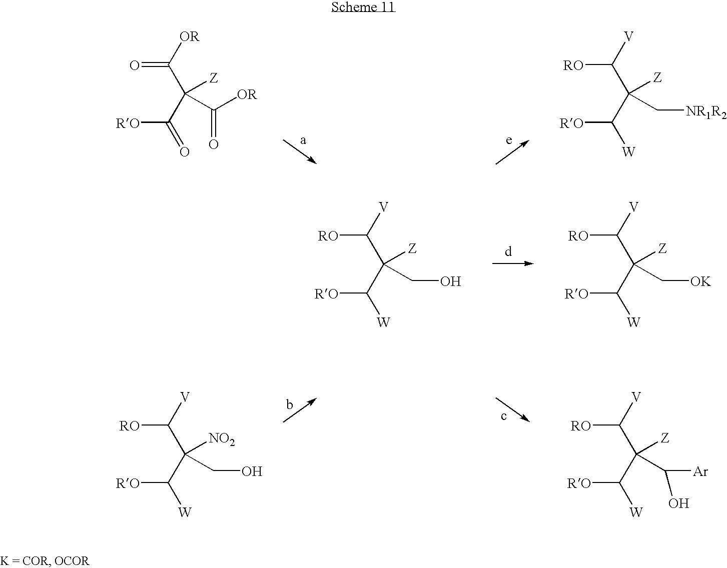

3. Annulated 1,3-propane diols

Compounds of Formula I wherein V and Z or V and W are connected by four carbons to form a ring can be prepared from a 1,3-cyclohexanediol. For example, cis, cis-1,3,5-cyclohexanetriol can be modified to give various other 1,3,5-cyclohexanetriols which are useful for the preparations of compounds of Formula I wherein R¹¹ and R¹¹ together are

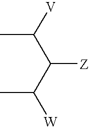

wherein together V and W are connected via 3 atoms to form a cyclic group containing 6 carbon atoms substituted with a hydroxy group. It is envisioned that these modifications can be performed either before or after formation of a cyclic phosphonate 1,3-propanediol ester. Various 1,3-cyclohexanediols can also be prepared using Diels-Alder reactions (e.g. using a pyrone as the diene: Tetrahedron Lett. 1991, 32, 5295). 2-Hydroxymethylcyclohexanols and 2-hydroxymethylcyclopentanols are useful for the preparations of compounds of Formula I wherein R¹¹ and R¹¹ together are

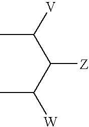

wherein together V and Z are connected via 2 or 3 atoms to form a cyclic group containing 5 or 6 carbon atoms. 1,3-Cyclohexanediol derivatives are also prepared via other cycloaddition reaction methodologies. For example, cycloadducts from the cycloadditon reactions of a nitrile oxide and an olefin can be converted to a 2-ketoethanol derivative which can be further converted to a 1,3-propanediol (including 1,3-cyclohexanediol, 2-hydroxymethylcyclohexanol and 2-hydroxymethylcyclopentanol) using known chemistry (J. Am. Chem. Soc. 107:6023 (1985)). Alternatively, precursors to 1,3-cyclohexanediol can be made from quinic acid (Tetrahedron Lett. 32:547 (1991)).

### EXAMPLES

The compounds used in this invention and their preparation can be understood further by the Examples, which illustrate some of the processes by which these compounds are prepared. These Examples should not however be construed as specifically limiting the invention, and variations of the compounds, now known or later developed, are considered to fall within the scope of the present invention as hereinafter claimed.

### Example 1

### Preparation of 5-[2-amino-5-(keto)thiazole-4-yl]furan-2-phosphonic acids

The synthesis of {5-[2-amino-5-(2,2-dimethylpropionyl)thiazol-4-yl]-furan-2-yl}phosphonic acid (1.1) is given to exemplify the general synthesis of this type of compound. 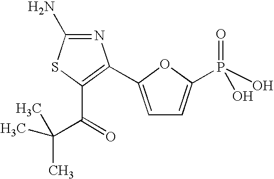
Step A

A solution of 2-furoic acid (1 mmol) in THF was added to a THF solution of LDA (lithium diisopropylamide, 2 mmole) at −78° C. and the resulting solution was stirred at −78° C. After 1 h the reaction mixture was treated with diethyl chlorophosphate (1.2 mmol), stirred at −78° C. for 1 h and at 25° C. for 12 h. The reaction mixture was quenched with saturated ammonium chloride. Extraction and chromatography gave 5-diethylphosphono-2-furoic acid as a yellow solid.

Step B

A solution of 5-diethylphosphono-2-furoic acid (1 mmole) and O-methyl-N-methylhydroxylamide HCl salt (1.3 mmole) in DMF was treated with triethylamine (2.2 mmole) and benzotriazol-1-yl-oxytripyrrolidinophosphonium hexafluorophosphate (PyBOP, 1.2 mmole) at 25° C. After 12 h, the reaction was subject to extraction and chromatography to give 5-diethylphosphono-2-(N-methyl-N-methoxy)furancarboxamide as a solid.

Step C

A solution of pinacolone (1.4 mmole) in THF was cooled to −78° C. and treated with n-BuLi (1.5 mmole). After 1 h, to the reaction was added a solution of 5-diethylphosphono-2-(N-methyl-N-methoxy)furancarboxamide (1 mmole) in THF and stirred at −78° C. for 1 h and at 25° C. for 12 h. The reaction was quenched with saturated ammonium chloride and subjected to extraction and chromatography to give 5-diethylphosphono-2-[1-(4,4-dimethyl-1,3-dioxo)pentyl]furan as an oil.

Step D

A solution of 5-diethylphosphono-2-[1-(4,4-dimethyl-1,3-dioxo)pentyl]furan (1 mmole) in carbon tetrachloride and ethanol was treated with copper (II) bromide (1.6 mmole) at 25° C. After heating at 70° C. for 3 h the reaction was cooled to 25° C. and subjected to extraction and chromatography to give 5-diethylphosphono-2-[1-(2-bromo-4,4-dimethyl-1,3-dioxo)pentyl]furan as an oil.

Step E

A solution of 5-diethylphosphono-2-[1-(2-bromo-4,4-dimethyl-1,3-dioxo)-pentyl]furan (1 mmole) in ethyl acetate and ethanol was treated with thiourea (1.8 mmole) at 25° C. After heating at 70° C. for 3 h the reaction was cooled to 25° C. and subjected to extraction and chromatography to give {5-[2-Amino-5-(2,2-dimethyl-propionyl)-thiazol-4-yl]-furan-2-yl}-phosphonic acid diethyl ester as a solid.

Step F

A solution of {5-[2-amino-5-(2,2-dimethyl-propionyl)thiazol-4-yl]furan-2-yl}phosphonic acid diethyl ester (1 mmole) in methylene chloride was treated with TMSBr (10 mmole) at 25° C. After 12 h the reaction was evaporated to dryness and the residue was suspended in acetone-water to give a yellow solid. The solid was collected via filtration and dried under vacuum to give {5-[2-amino-5-(2,2-dimethyl-propionyl)thiazol-4-yl]-furan-2-yl}phosphonic acid (1.1) as a solid. Mp>220° C. Anal. calcd. for C₁₂H₁₅N₂O₅PS: C, 43.64; H, 4.58; N, 8.48. Found: C, 43.47; H, 4.64; N, 8.55. According to the above procedures or in some cases with minor modifications of these procedures using conventional chemistry the following compounds were prepared:

(1.2) {5-[2-amino-5-(2,2-dimethyl-butyryl)thiazol-4-yl]furan-2-yl}phosphonic acid. Mp>220° C. Anal. calcd. for C₁₃H₁₇N₂O₅PS: C, 45.35; H, 4.98; N, 8.14. Found: C, 45.13; H, 5.33; N, 8.00.

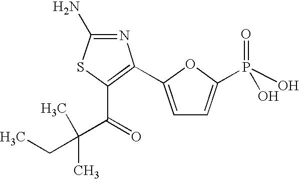

(1.3) [5-(2-amino-5-(2-ethyl-2-methyl-butyryl)thiazol-4-yl)furan-2-yl]phosphonic acid. Mp 202-205° C. Anal. calcd. for C₁₄H₁₉N₂O₅PS+0.2H₂O: C, 46.46; H, 5.40; N, 7.74. Found: C, 5.31; N, 7.77.

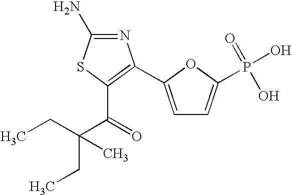

(1.4) [5-(2-amino-5-acetylthiazol-4-yl)furan-2-yl]phosphonic acid. Mp>207-212° C. Anal. calcd. for C₉H₉N₂O₅PS+0.2H₂O: C, 37.04; H, 3.25; N, 9.60. Found: C, 37.14; H, 3.54; N, 9.32.

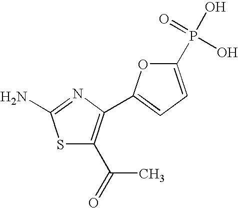

(1.5) [5-(2-amino-5-benzoylthiazol-4-yl)furan-2-yl]phosphonic acid. Mp>210° C. Anal. calcd. for C₁₄H₁₁N₂O₅PS: C, 48.00; H, 3.17; N, 8.00. Found: C, 47.63; H, 2.88; N, 7.84.

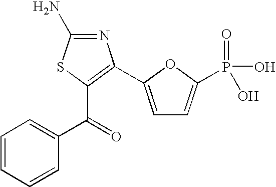

(1.6) [5-(2-amino-5-cyclohexylcarbonyl-thiazol-4-yl)furan-2-yl]-phosphonic acid. Anal. calcd. for C₁₄H₁₇N₂O₅PS+1.3H₂O: C, 44.28; H, 5.20; N, 7.38. Found: C, 44.14; H, 5.02; N, 7.21.

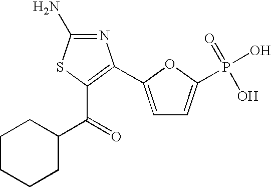

(1.7) [5-(2-amino-5-(2-thienylcarbonyl)thiazol-4-yl)furan-2-yl]-phosphonic acid. Anal. calcd. for C₁₂H₉N₂O₅PS₂: C, 40.45; H, 2.55; N, 7.86. Found: C, 40.23; H, 2.28; N, 7.84.

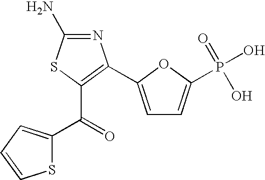

(1.8) [5-(2-amino-5-(3-fluorobenzoyl)thiazol-4-yl)furan-2-yl]-phosphonic acid. Anal. calcd. for C₁₄H₁₀N₂O₅ PFS+0.5MeOH: C, 45.32; H, 3.15; N, 7.29. Found: C, 45.61; H, 3.52; N, 7.09.

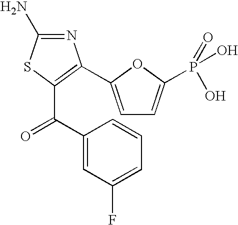

(1.9) [5-(2-amino-5-(4-chlorobenzoyl)thiazol-4-yl)furan-2-yl]-phosphonic acid. Anal. calcd. for C₁₄H₁₀N₂O₅PSCl: C, 43.71; H, 2.62; N, 7.28. Found: C, 43.47; H, 2.76; N, 7.18.

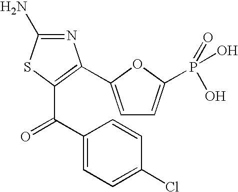

(1.10) [5-(2-amino-5-(4-methylbenzoyl)thiazol-4-yl)furan-2-yl]-phosphonic acid. Anal. calcd. for C₁₅H₁₃N₂O₅PS+0.3H₂O: C, 48.73; H, 3.71; N, 7.58. Found: C, 48.75; H, 3.64; N, 7.55.

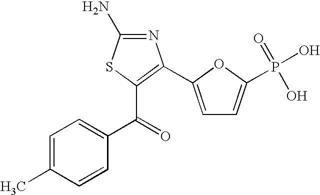

(1.11) [5-(2-amino-5-(3-methylbenzoyl)thiazol-4-yl)furan-2-yl]-phosphonic acid. Anal. calcd. for C₁₅H₁₃N₂O₅PS+0.35H₂O: C, 48.61; H, 3.73; N, 7.56. Found: C, 48.63; H, 3.53; N, 7.61.

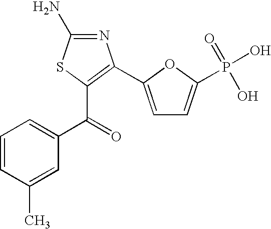

(1.12) [5-(2-amino-5-(3-chlorobenzoyl)thiazol-4-yl)furan-2-yl]-phosphonic acid. Anal. calcd. for C₁₄H₁₀N₂O₅PSCl+0.2H₂O+0.1EtOAc: C, 43.55; H, 2.84; N, 7.05. Found: C, 43.34; H, 3.00; N, 6.89.

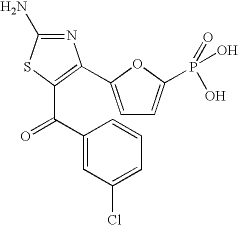

(1.13) [5-(2-amino-5-(2-methylbenzoyl)thiazol-4-yl)furan-2-yl]-phosphonic acid. Anal. calcd. for C₁₅H₁₃N₂O₅PS+0.05 HBr: C, 48.91; H, 3.57; N, 7.60. Found: C, 48.88; H, 3.22; N, 7.20.

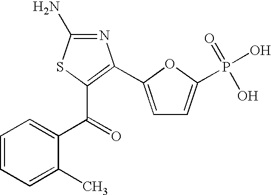

(1.14) [5-(2-amino-5-(2-methoxybenzoyl)thiazol-4-yl)furan-2-yl]-phosphonic acid. Anal. calcd. for C₁₅H₁₃N₂O₆PS+1.5H₂O: C, 44.23; H, 3.96; N, 6.88. Found: C, 44.26; H, 3.84; N, 6.87.

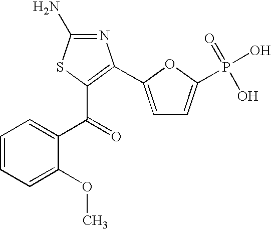

(1.15) [5-(2-amino-5-(2-chlorobenzoyl)thiazol-4-yl)furan-2-yl]-phosphonic acid. Anal. calcd. for C₁₄H₁₀N₂O₅PSCl: C, 43.71; H, 2.62; N, 7.28. Found: C, 43.33; H, 3.00; N, 6.92.

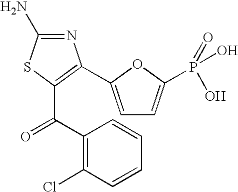

(1.16) [5-(2-amino-5-(4-methoxybenzoyl)thiazol-4-yl)furan-2-yl]-phosphonic acid. Anal. calcd. for C₁₅H₁₃N₂O₆PS+0.25H₂O: C, 46.82; H, 3.54; N, 7.28. Found: C, 46.95; H, 3.80; N, 6.99.

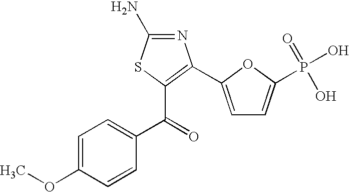

(1.17) [5-(2-amino-5-(3,4-dimethoxybenzoyl)thiazol-4-yl)furan-2-yl]-phosphonic acid. Anal. calcd. for C₁₆H₁₅N₂O₇PS: C, 47.67; H, 4.44; N, 6.56. Found: C, 46.83; H, 3.68;

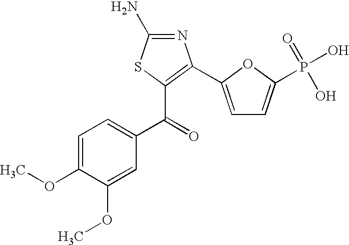

(1.18) [5-(2-amino-5-(3-methoxybenzoyl)thiazol-4-yl)furan-2-yl]-phosphonic acid. Anal. calcd. for C₁₅H₁₃N₂O₆PS: C, 47.37; H, 3.45; N, 7.37. Found: C, 48.46; H, 3.83; N, 7.68.

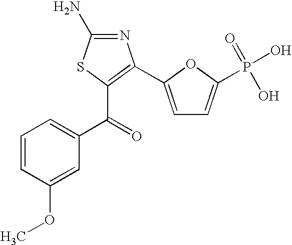

(1.19) [5-(2-amino-5-(3,4-methylenedioxybenzoyl)thiazol-4-yl)-furan-2-yl]phosphonic acid. Anal. calcd. for C₁₅H₁₁N₂O₇PS: C, 45.69; H, 2.81; N, 7.10. Found: C, 45.32; H, 3.20; N, 6.94.

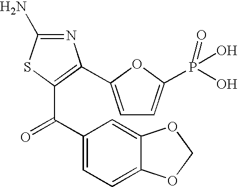

(1.20) [5-(2-amino-5-(3-pyridylcarbonyl)thiazol-4-yl)furan-2-yl]-phosphonic acid. Anal. calcd. for C₁₃H₁₀N₃O₅PS+1.5H₂O: C, 41.27; H, 3.46; N, 11.11. Found: C, 41.37; H, 3.36; N, 11.06.

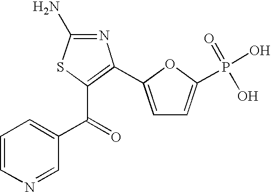

(1.21) [5-(2-amino-5-(3-chloro-4-(1-pyrrolidinyl)benzoyl)-thiazol-4-yl)furan-2-yl]-phosphonic acid. Anal. calcd. for C₁₈H₁₇N₃O₅PSCl+0.8H₂O: C, 46.17; H, 4.00; N, 8.97. Found: C, 46.16; H, 4.16; N, 8.86.

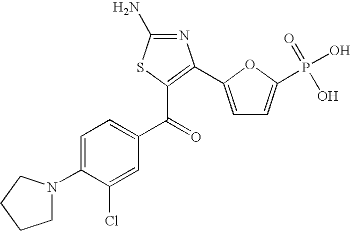

(1.22) [5-(2-amino-5-(4-fluorobenzoyl)thiazol-4-yl)furan-2-yl]-phosphonic acid. Anal. calcd. for C₁₄H₁₀N₂O₅PSF+1.0H₂O+0.2HBr: C, 41.78; H, 3.06; N, 6.96. Found: C, 41.92; H, 3.48; N, 6.83.

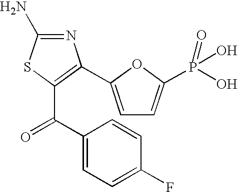

(1.23) [5-(2-amino-5-(2-ethylbutyryl)thiazol-4-yl)furan-2-yl]-phosphonic acid. Anal. calcd. for C₁₃H₁₇N₂O₅PS: C, 45.35; H, 4.98; N, 8.14. Found: C, 44.96; H, 5.08; N, 7.83.

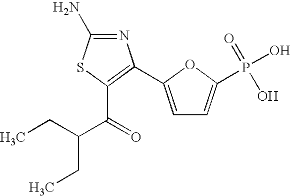

(1.24) [5-(2-amino-5-(4-trifluoromethylbenzoyl)thiazol-4-yl)-furan-2-yl]phosphonic acid. Anal. calcd. for C₁₅H₁₀N₂O₅PSF₃: C, 43.07; H, 2.41; N, 6.70. Found: C, 42.82; H, 2.80; N, 6.54.

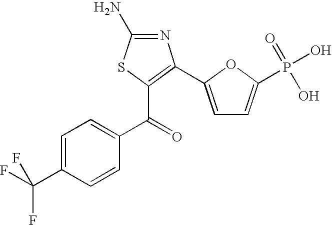

(1.25) [5-(2-amino-5-(3-chloro-4-(1-morpholinyl)benzoyl)-thiazol-4-yl)furan-2-yl]phosphonic acid. ¹H NMR (CD₃OD), δ 7.72, 7.7, 7.2, 6.92, 3.8, 3.12 ppm.

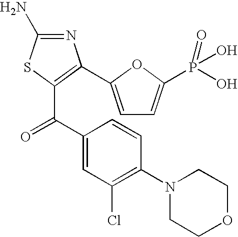

(1.26) [5-(2-amino-5-(3-trifluoromethylbenzoyl)thiazol-4-yl)-furan-2-yl]phosphonic acid. Anal. calcd. for C₁₅H₁₀N₂O₅PSF₃: C, 43.07; H, 2.41; N, 6.70. Found: C, 43.46; H, 2.80; N, 6.45.

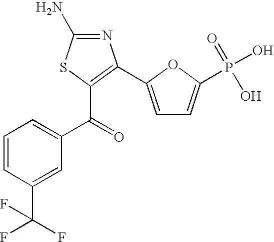

(1.27) [5-(2-amino-5-(2-trifluoromethylbenzoyl)thiazol-4-yl)-furan-2-yl]phosphonic acid. Anal. calcd. for C₁₅H₁₀N₂O₅PSF₃+0.5H₂O: C, 42.16; H, 2.59; N, 6.56. Found: C, 42.42; H, 3.23; N, 6.31.

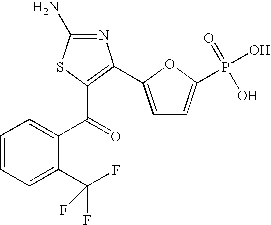

(1.28) [5-(2-amino-5-(4-phenylbenzoyl)thiazol-4-yl)furan-2-yl]-phosphonic acid. Anal. calcd. for C₂₀H₁₅N₂O₅PS: C, 56.34; H, 3.55; N, 6.57. Found: C, 56.11; H, 3.75; N, 6.38.

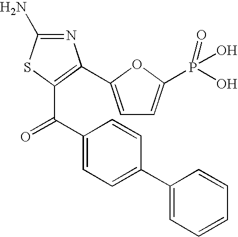

(1.29) [5-(2-amino-5-(2-naphthylcarbonyl)thiazol-4-yl)furan-2-yl]-phosphonic acid. Anal. calcd. for C₁₈H₁₃N₂O₅PS+0.7H₂O: C, 52.35; H, 3.51; N, 6.78. Found: C, 52.15; H, 3.55; N, 6.45.

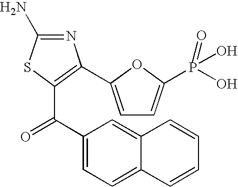

(1.30) [5-(2-amino-5-cyclopentylcarbonylthiazol-4-yl)furan-2-yl]-phosphonic acid. Anal. calcd. for C₁₃H₁₅N₂O₅PS+1.2H₂O: C, 42.90; H, 4.82; N, 7.70. Found: C, 43.04; H, 5.19; N, 7.51.

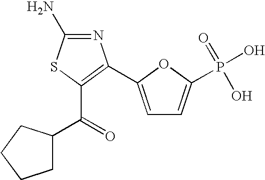

(1.31) [5-(2-amino-5-(4-piperidinylbenzoyl)thiazol-4-yl)furan-2-yl]-phosphonic acid. Anal. calcd. for C₁₉H₂₀N₃O₅PS+0.2 HBr: C, 50.76; H, 4.53; N, 9.35. Found: C, 50.63; H, 4.65; N, 9.21.

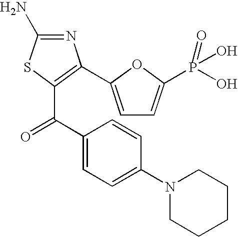

(1.32) [5-(2-amino-5-(4-(N,N-dimethylamino)benzoyl)thiazol-4-yl)-furan-2-yl]phosphonic acid. Anal. calcd. for C₁₆H₁₆N₃O₅PS: C, 48.86; H, 4.10; N, 10.68. Found: C, 46.14; H, 5.46; N, 9.02.

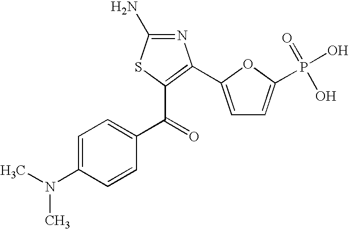

(1.33) [5-(2-amino-5-(2-methylbutyryl)thiazol-4-yl)furan-2-yl]-phosphonic acid. Anal. calcd. for C₁₂H₁₅N₂O₅PS: C, 43.64; H, 4.58; N, 8.48. Found: C, 43.47; H, 4.85; N, 8.29.

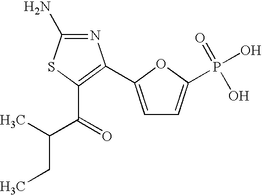

(1.34) [5-(2-amino-5-cyclobutylcarbonylthiazol-4-yl)furan-2-yl]-phosphonic acid. Anal. calcd. for C₁₂H₁₃N₂O₅PS+0.2H₂O: C, 43.43; H, 4.07; N, 8.44. Found: C, 43.49; H, 4.20; N, 8.28.

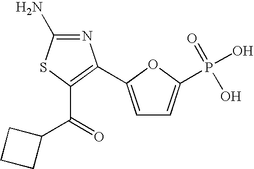

### Example 2

### Preparation of Phosphoramides as Prodrugs

Step A. A solution of {5-[2-amino-5-(2,2-dimethyl-propionyl)thiazol-4-yl]furan-2-yl}phosphonic acid (1.1) (1 mmole), DMF (1.2 mmole) and oxalyl chloride (4 mmole) in 1,2-dichloroethane was heated at 50° C. for 2 h. The reaction solution was evaporated to dryness and the residue was redissolved in 1,2-dichloroethane. After cooling to 0° C., 2-methylalanine ethyl ester (3.5 mmole) and N,N-diethylisopropylamine (3.5 mmole) were added. After stirring at 25° C. for 12 h, the reaction was subjected to extraction and chromatography to give 2-(dimethylaminomethyleneamino)-5-(2,2-dimethylpropionyl)-4-{2-[5-(N,N′-2-ethoxycarbonylprop-2-yl)-phosphon-amido]-furanyl}thiazole.

Step B. A solution of 2-(dimethylamino-methyleneamino)-5-(2,2-dimethylpropionyl)-4-{[5-(N,N′-2-ethoxycarbonylprop-2-yl)phosphonamido]furan-2-yl}thiazole (1 mmole) in acetic acid and isopropanol was heated to 85° C. After 12 h the reaction was subjected to extraction and chromatography to give 2-amino-5-(2,2-dimethylpropionyl)-4-{[5-(N,N′-(2-ethoxy-carbonylprop-2-yl)phosphonamido]furan-2-yl}thiazole (2.1) as a yellow solid. Mp 149-152° C. Anal. Calcd for C₂₄H₃₇N₄O₇PS: C, 51.79; H, 6.70; N, 10.07. Found: C, 51.39; H, 6.51; N, 10.26.

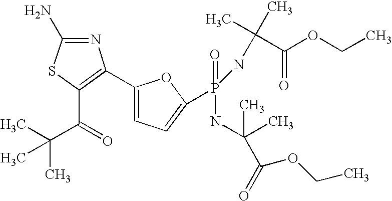

The following compounds were prepared according to the above described procedures or in some cases with minor modifications of these procedures using conventional chemistry:

(2.2) 2-amino-5-(2,2-dimethylpropionyl)-4-{[5-(N,N′-((S)-1-ethoxycarbonyl)-ethyl)phosphonamido]furan-2-yl}thiazole. Foam. Anal. Calcd for C₂₂H₃₃N₄O₇PS+0.4H₂O: C, 49.32; H, 6.36; N, 10.46. Found: C, 49.17; H, 6.56; N, 10.61.

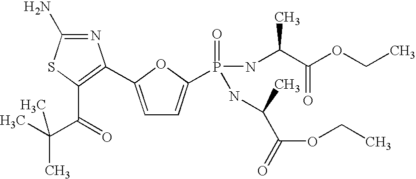

(2.3) 2-amino-5-(2,2-dimethylpropionyl)-4-{[5-(N,N′-(2-isopropyloxy-carbonyl-prop-2-yl)phosphonamido]furan-2-yl}thiazole. Foam. Anal. Calcd for C₂₆H₄₁N₄O₇PS: C, 53.41; H, 7.07; N, 9.58. Found: C, 53.20; H, 6.81; N, 9.36.

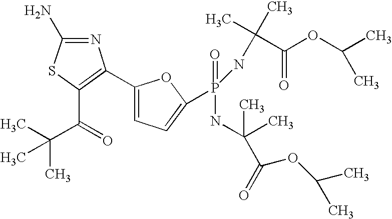

(2.4) 2-amino-5-(2,2-dimethylpropionyl)-4-{[5-[(N,N′-ethoxycarbonylmethyl)-phosphonamido]furan-2-yl}thiazole. Mp 86-88° C. Anal. Calcd for C₂₀H₂₉N₄O₇PS: C, 48.00; H, 5.84; N: 11.19. Found: C, 47.88; H, 5.93; N: 11.16.

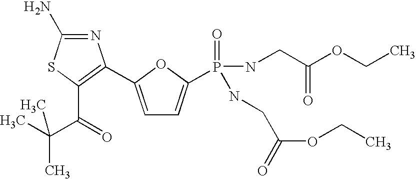

(2.5) 2-amino-5-(2-ethyl-2-methylbutyryl)-4-{[5-(N,N′-(2-ethoxycar-bonyl-prop-2-yl)phosphonamido]furan-2-yl}thiazole. Mp 157-160° C. Anal. Calcd for C₂₆H₄₁N₄O₇PS+0.25H₂O: C, 53.00; H, 7.10; N, 9.51. Found: C, 53.18; H, 6.70; N, 9.11.

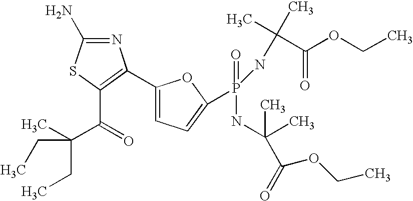

(2.6) 2-amino-5-(2-ethyl-2-methylbutyryl)-4-{[5-(N,N′-(2-isopropyloxy-carbonyl-prop-2-yl)phosphonamido]furan-2-yl}thiazole. Mp 160-164° C. Anal. Calcd for C₂₈H₄₅N₄O₇PS+0.18H₂O: C, 54.60; H, 7.42; N, 9.10. Found: C, 54.99; H, 7.23; N, 8.67.

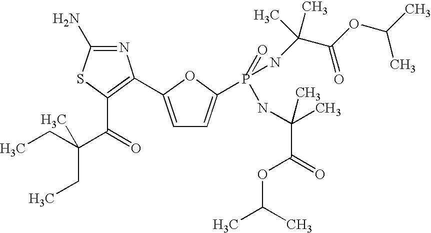

(2.7) 2-amino-5-(2-ethyl-2-methylbutyryl)-4-{[5-(N,N′-((S)-1-ethoxycar-bonyl)ethyl)phosphonamido]furan-2-yl}thiazole. Foam. Anal. Calcd for C₂₄H₃₇N₄O₇PS: C, 51.79; H, 6.70; N, 10.07. Found: C, 51.84; H, 6.78; N, 9.76.

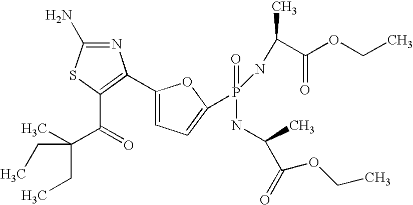

(2.8) 2-amino-5-(2,2-dimethylbutyryl)-4-{[5-(N,N′-(2-isopropyloxycar-bonyl-prop-2-yl)phosphonamido]furan-2-yl}thiazole. Foam. Anal. Calcd for C₂₇H₄₃N₄O₇PS: C, 54.17; H, 7.24; N, 9.36. Found: C, 53.92; H, 7.38; N, 9.11.

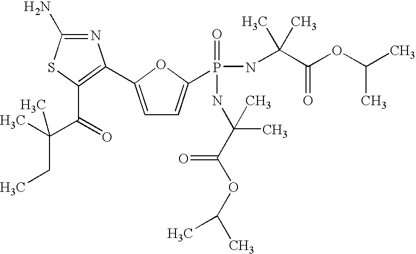

(2.9) 2-amino-5-(2,2-dimethylbutyryl)-4-{[5-(N,N′-(2-ethoxycarbonyl-prop-2-yl)phosphonamido]furan-2-yl}thiazole. Foam. Anal. Calcd for C₂₅H₃₉N₄O₇PS: C, 52.62; H, 6.89; N, 9.82. Found: C, 52.54; H, 6.50; N, 10.12.

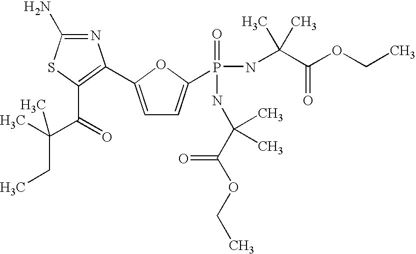

(2.10) 2-amino-5-(2,2-dimethylbutyryl)-4-{[5-(N,N′-((S)-1-ethoxycarbonyl)-ethyl)phosphonamido]furan-2-yl}thiazole. Foam. Anal. Calcd for C₂₃H₃₅N₄O₇PS: C, 50.91; H, 6.50; N, 10.33. Found: C, 50.56; H, 6.88; N, 10.47.

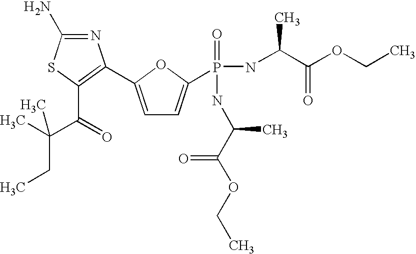

(2.11) 2-amino-5-(2-ethyl-2-methylbutyryl)-4-{[5-(N,N′-((S)-1-isopropyloxy-carbonyl)-ethyl)phosphonamido]furan-2-yl}thiazole. Foam. Anal. Calcd for C₂₆H₄₁N₄O₇PS+0.6H₂O: C, 52.44; H, 7.14; N, 9.41. Found: C, 52.31; H, 6.21; N, 9.21.

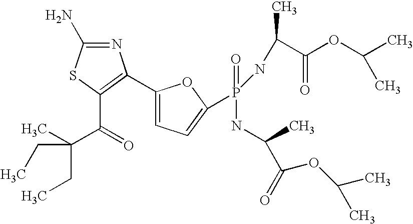

(2.12) 2-amino-5-(2,2-dimethylbutyryl)-4-{[5-(N,N′-tert-butyloxycarbonyl-methyl)phosphonamido]furan-2-yl}thiazole. Foam. Anal. Calcd for C₂₅H₃₉N₄O₇PS+0.4 EtOAc: C, 52.70; H, 6.99; N, 9.38. Found: C, 52.32; H, 6.86; N, 9.61.

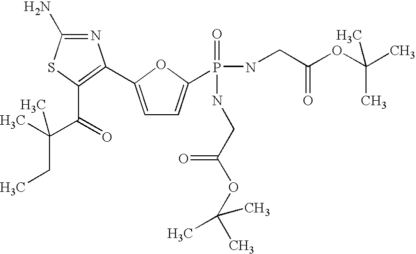

(2.13) 2-amino-5-(2,2-dimethylpropionyl)-4-{[5-(N,N′-((S)-1-isopropyl-oxycarbonyl)ethyl)-phosphonamido]furan-2-yl}thiazole. Foam. Anal. Calcd for C₂₄H₃₇N₄O₇PS+0.1H₂O: C, 51.62; H, 6.71; N, 10.03. Found: C, 51.30; H, 6.97; N, 10.29.

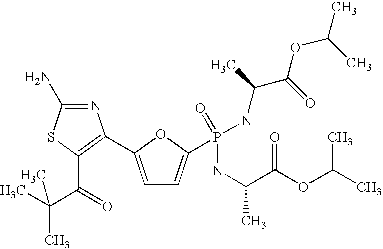

(2.14) 2-amino-5-(2-methylbenzoyl)-4-{[5-(N,N′-((S)-1-ethyl-oxycarbonyl)ethyl)-phosphonamido]furan-2-yl}thiazole. Foam. Anal. Calcd for C₂₅H₃₁N₄O₇PS: C, 53.37; H, 5.55; N, 9.96. Found: C, 53.33; H, 5.58; N, 9.85.

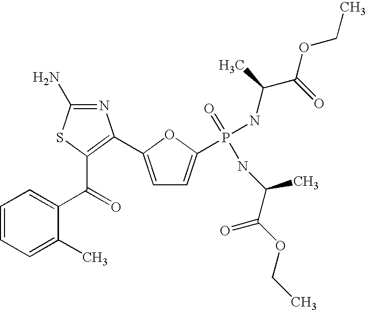

(2.15) 2-amino-5-(2-methylbenzoyl)-4-{[5-(N,N′-(2-ethoxycarbonyl-prop-2-yl)phosphonamido]furan-2-yl}thiazole. Foam. Anal. Calcd for C₂₇H₃₅N₄O₇PS: C, 54.91; H, 5.97; N, 9.49. Found: C, 54.64; H, 5.85; N, 9.48.

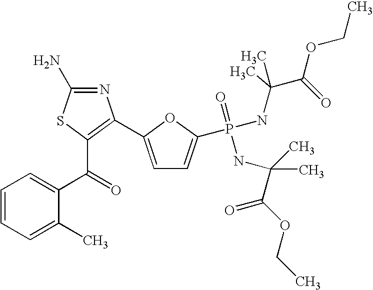

(2.16) 2-amino-5-(4-methylbenzoyl)-4-{[5-(N,N′-((S)-1-ethyl-oxycarbonyl)ethyl)-phosphonamido]furan-2-yl}thiazole. Foam. Anal. Calcd for C₂₅H₃₁N₄O₇PS: C, 53.37; H, 5.55; N, 9.96. Found: C, 53.02; H, 5.52; N, 9.89.

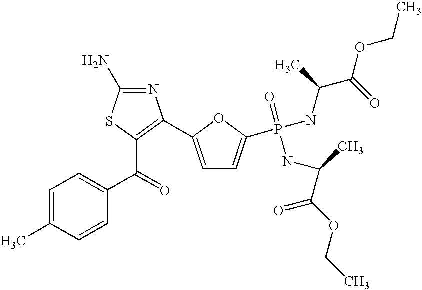

(2.17) 2-amino-5-(4-methylbenzoyl)-4-{[5-(N,N′-(2-ethoxycarbonyl-prop-2-yl)phosphonamido]furan-2-yl}thiazole. Foam. Anal. Calcd for C₂₇H₃₅N₄O₇PS: C, 54.91; H, 5.97; N, 9.49. Found: C, 54.51; H, 5.93; N, 9.35.

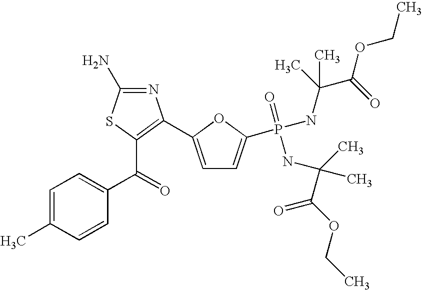

(2.18) 2-amino-5-(3-fluorobenzoyl)-4-{[5-(N,N′-((S)-1-isopropyl-oxycarbonyl)ethyl)-phosphonamido]furan-2-yl}thiazole. Foam. Anal. Calcd for C₂₆H₃₂N₄O₇PSF+0.2 CH₂Cl₂: C, 51.45; H, 5.34; N, 9.16. Found: C, 51.30; H, 5.25; N, 8.97.

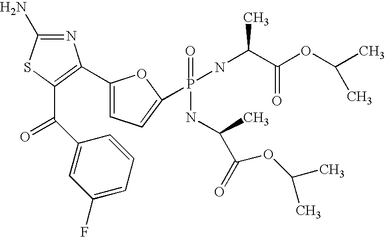

(2.19) 2-amino-5-(3-fluorobenzoyl)-4-{[5-(N,N′-((S)-1-ethyloxy-carbonyl)ethyl)-phosphonamido]furan-2-yl}thiazole. Foam. Anal. Calcd for C₂₆H₃₂N₄O₇PSF+0.4 CH₂Cl₂: C, 48.80; H, 4.83; N, 9.33. Found: C, 48.81; H, 4.51; N, 8.92.

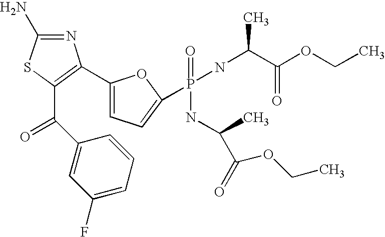

(2.20) 2-amino-5-(4-methylbenzoyl)-4-{[5-(N,N′-(2-isopropyloxycarbonyl-prop-2-yl)phosphonamido]furan-2-yl}thiazole. Foam. Anal. Calcd for C₂₉H₃₉N₄O₇PS: C, 56.30; H, 6.35; N, 9.06. Found: C, 55.96; H, 6.08; N, 9.11.

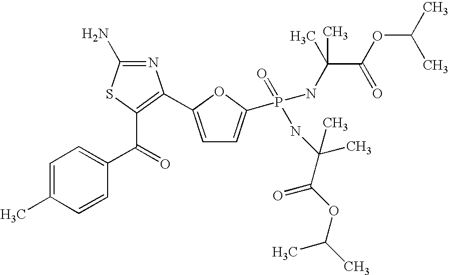

(2.21) 2-amino-5-(2-methylbenzoyl)-4-{[5-(N,N′-(2-isopropyloxycarbonyl-prop-2-yl)phosphonamido]furan-2-yl}thiazole. Foam. Anal. Calcd for C₂₉H₃₉N₄O₇PS: C, 56.30; H, 6.35; N, 9.06. Found: C, 55.90; H, 6.21; N, 9.08.

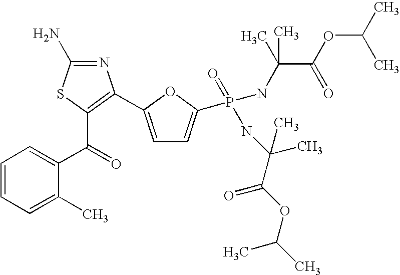

(2.22) 2-amino-5-(2-methylbenzoyl)-4-{[5-(N,N′-((S)-1-isopropyl-oxycarbonyl)ethyl)-phosphonamido]furan-2-yl}thiazole. Foam. Anal. Calcd for C₂₇H₃₅N₄O₇PSF: C, 54.91; H, 5.97; N, 9.49. Found: C, 54.85; H, 6.10; N, 9.55.

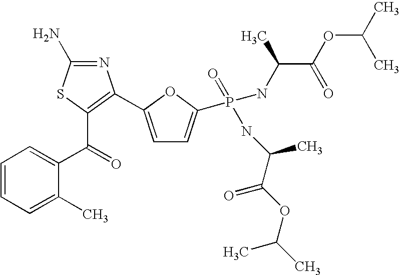

(2.23) 2-amino-5-(2-ethylbutyryl)-4-{[5-(N,N′-(2-ethyloxycarbonyl-prop-2-yl)phosphonamido]furan-2-yl}thiazole. Foam. Anal. Calcd for C₂₅H₃₉N₄O₇PS: C, 52.62; H, 6.89; N, 9.82. Found: C, 42.28; H, 5.74; N, 7.82.

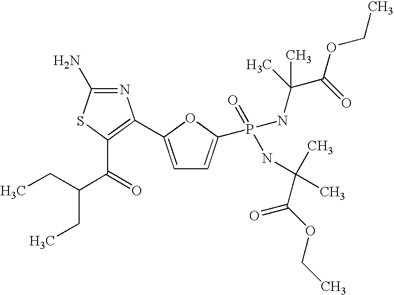

(2.24) 2-amino-5-(2-ethylbutyryl)-4-{[5-(N,N′-((S)-1′-ethyl-oxycarbonyl)ethyl)-phosphonamido]furan-2-yl}thiazole. Foam. ¹H NMR (CDCl₃), δ 8.15, 7.01, 4.18, 4.00, 2.60, 1.75, 1.52, 1.42, 1.22, 0.82 ppm.

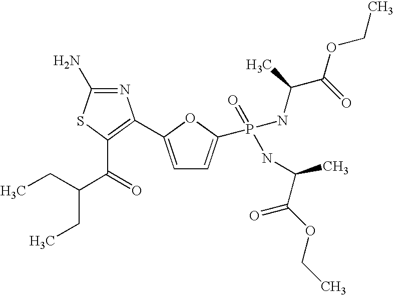

(2.25) 2-amino-5-(2-ethylbutyryl)-4-{[5-(N,N′-(2-isopropyloxycarbonyl-prop-2-yl)phosphonamido]furan-2-yl}thiazole. Foam. Anal. Calcd for C₂₇H₄₃N₄O₇PS: C, 54.17; H, 7.24; N, 9.36. Found: C, 53.99; H, 7.35; N, 9.45.

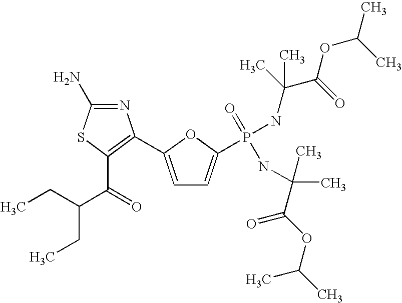

(2.26) 2-amino-5-(2-ethylbutyryl)-4-{[5-(N,N′-((S)-1-isopropyl-oxycarbonyl)ethyl)-phosphonamido]furan-2-yl}thiazole. Foam. Anal. Calcd for C₂₅H₃₉N₄O₇PS: C, 52.62; H, 6.89; N, 9.82. Found: C, 52.29; H, 7.12; N, 9.79.

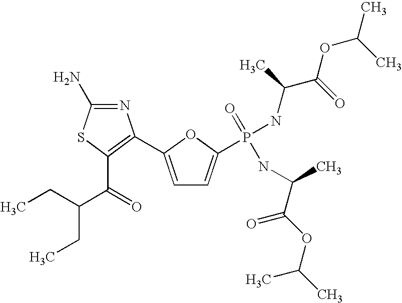

(2.27) 2-amino-5-(3-fluorobenzoyl)-4-{[5-(N,N′-(2-ethyloxycarbonyl-prop-2-yl)phosphonamido]furan-2-yl}thiazole. Foam. Anal. Calcd for C₂₆H₃₂N₄O₇PSF+0.1 CH₂Cl₂: C, 51.98; H, 5.38; N, 9.29. Found: C, 51.60; H, 5.03; N, 9.31.

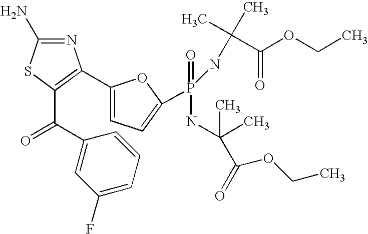

(2.28) 2-amino-5-(3-fluorobenzoyl)-4-{[5-(N,N′-(2-isopropyloxycarbonyl-prop-2-yl)phosphonamido]furan-2-yl}thiazole. Foam. Anal. Calcd for C₂₆H₃₂N₄O₇P SF: C, 54.01; H, 5.83; N, 9.00. Found: C, 53.92; H, 5.62; N, 8.76.

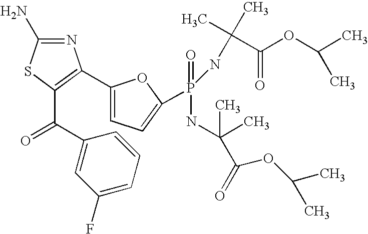

(2.29) 2-amino-5-cyclobutylcarbonyl-4-{[5-(N,N′-(2-ethyloxycarbonyl-prop-2-yl)phosphonamido]furan-2-yl}thiazole. Foam. Anal. Calcd for C₂₄H₃₅N₄O₇PS: C, 51.98; H, 6.36; N, 10.10. Found: C, 51.63; H, 5.98; N, 9.93.

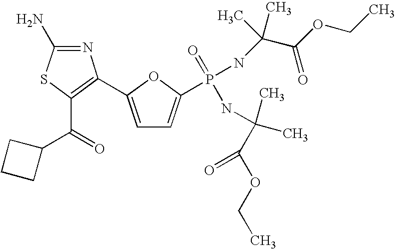

(2.30) 2-amino-5-cyclobutylcarbonyl-4-{[5-(N,N′-((S)-1-ethyl-oxycarbonyl)ethyl)-phosphonamido]furan-2-yl}thiazole. Foam. Anal. Calcd for C₂₂H₃₁N₄O₇PS+0.2 EtOAc: C, 50.29; H, 6.01; N, 10.38. Found: C, 50.39; H, 5.74; N, 10.00.

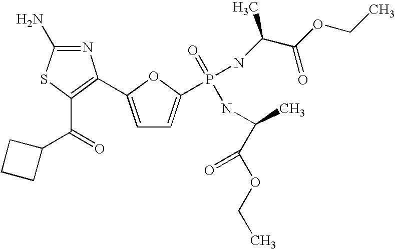

(2.31) 2-amino-5-cyclobutylcarbonyl-4-{[5-(N,N′-((S)-1-isopropyl-oxycarbonyl)ethyl)-phosphonamido]furan-2-yl}thiazole. Foam. Anal. Calcd for C₂₄H₃₅N₄O₇PS: C, 51.98; H, 6.36; N, 10.10. Found: C, 51.59; H, 6.03; N, 9.78.

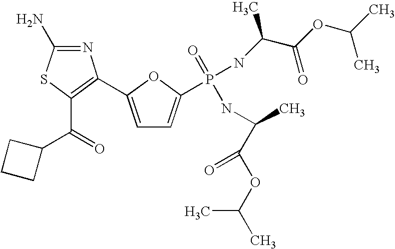

(2.32) 2-amino-5-cyclobutylcarbonyl-4-{[5-(N,N′-(2-isopropyloxycarbonyl-prop-2-yl)phosphonamido]furan-2-yl}thiazole. Foam. Anal. Calcd for C₂₆H₃₉N₄O₇PS: C, 53.60; H, 6.75; N, 9.62. Found: C, 53.47; H, 6.38; N, 9.56.

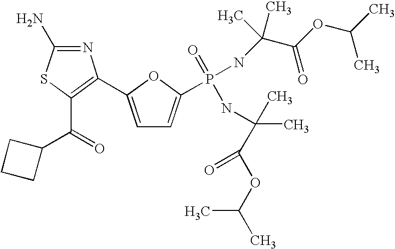

### Example 5

### Preparation of Mixed Phosphonate Esters and Phosphoramides as Prodrugs

Step A. A solution of {5-[2-amino-5-(2,2-dimethyl-propionyl)thiazol-4-yl]furan-2-yl}phosphonic acid (1.1) (1 mmole) and thionyl chloride (4 mmole) in 1,2-dichloroethane was heated at 50° C. for 2 h. The reaction solution was evaporated to dryness and the residue was redissolved in 1,2-dichloroethane. After cooling to 0° C., glycolate ethyl ester (0.9 mmole) and N,N-diethylisopropylamine (3.5 mmole) were added. After 1 h, 2-methylalanine ethyl ester (2 mmole) was added. After stirring at 25° C. for 12 h, the reaction was subjected to extraction and chromatography to give 2-amino-5-(2,2-dimethylpropionyl)-4-{2-[5-(N-(2-ethoxycarbonylprop-2-yl)-O-(ethoxycarbonylmethyl)monophosphonamido]furanyl}thiazole (5.2). Foam. Anal. Calcd for C₂₂H₃₂N₃O₈PS+0.1 MeCN: C, 49.97; H, 6.10; N, 8.14. Found: C, 50.34; H, 5.98; N, 8.30.

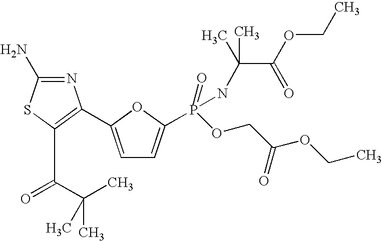

The following compounds were prepared according to the above described procedures or in some cases with minor modifications of these procedures using conventional chemistry:

2-amino-5-(2-ethylbutyryl)-4-{2-[5-(N-((S)-1-ethoxycarbonyl)ethyl)-O-(3,4-ethylenedioxyphenyl)monophosphonamido]furanyl}thiazole (5.1). Foam. Anal. Calcd for C₂₅H₃₀N₃O₈PS+0.1 MeOH: C, 53.19; H, 5.41; N, 7.41. Found: C, 52.89; H, 5.22; N, 7.82.

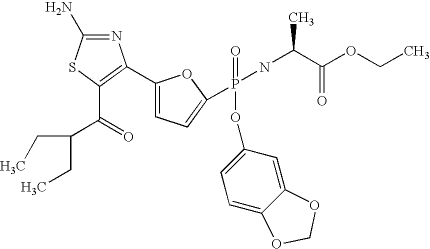

2-amino-5-(2,2-dimethylpropionyl)-4-{2-[5-(N-(2-isopropyloxy-carbonylprop-2-yl)-O-(3,4-ethylenedioxyphenyl)monophosphonamido]furanyl}thiazole (5.3). Foam. Anal. Calcd for C₂₅H₃₀N₃O₈PS: C, 53.28; H, 5.37; N, 7.46. Found: C, 53.12; H, 5.59; N, 7.57.

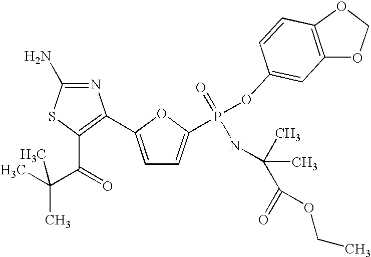

2-amino-5-cyclobutylcarbonyl-4-{2-[5-(N-(2-ethoxycarbonyl-prop-2-yl)-O-(ethoxycarbonylmethyl)monophosphonamido]furanyl}thiazole (5.4). Foam. Anal. Calcd for C₂₂H₃₀N₃O₈PS: C, 50.09; H, 5.73; N, 7.97. Found: C, 49.77; H, 5.85; N, 7.86.

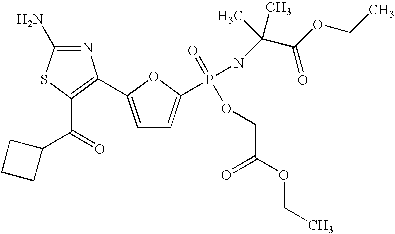

2-amino-5-(2,2-dimethylpropionyl)-4-{2-[5-(N-(2-ethoxycarbonyl-prop-2-yl)-O-(benzyloxycarbonylmethyl)monophosphonamido]furanyl}-thiazole (5.5). Foam. Anal. Calcd for C₂₇H₃₄N₃O₈PS+0.2H₂O: C, 54.48; H, 5.83; N, 7.06. Found: C, 54.18; H, 6.15; N, 7.02.

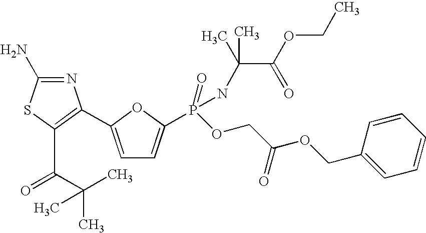

2-amino-5-(2,2-dimethylpropionyl)-4-{2-[5-(N-((S)-1-isopropyloxy-carbonyl)ethyl)-O-(ethoxycarbonylmethyl)monophosphonamido]furanyl}-thiazole (5.6). Foam. Anal. Calcd for C₂₂H₃₂N₃O₈PS+0.3H₂O: C, 49.40; H, 6.14; N, 7.85. Found: C, 49.04; H, 6.43; N, 7.57.

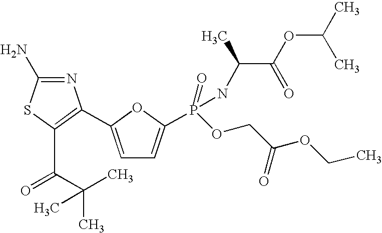

2-amino-5-(2,2-dimethylpropionyl)-4-{2-[5-(N-(2-isopropyloxy-carbonylprop-2-yl)-O-(ethoxycarbonylmethyl)monophosphonamido]furanyl}-thiazole (5.7). Foam. Anal. Calcd for C₂₃H₃₄N₃O₈PS+0.1 CH₂Cl₂: C, 50.26; H, 6.24; N, 7.61. Found: C, 49.96; H, 5.93; N, 7.55.

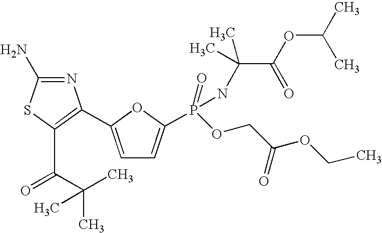

2-amino-5-(2,2-dimethylpropionyl)-4-{2-[5-(N-((S)-1-ethoxy-carbonyl)ethyl)-O-(ethoxycarbonylmethyl)monophosphonamido]furanyl}-thiazole (5.8). Foam. Anal. Calcd for C₂₁H₃₀N₃O₈PS: C, 48.93; H, 5.87; N, 8.15. Found: C, 48.62; H, 5.63; N, 8.11.

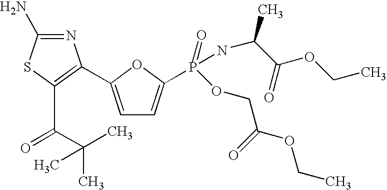

2-amino-5-(2,2-dimethylpropionyl)-4-{2-[5-(N-(1-ethoxycarbonyl-prop-2-yl)-O-(((S)-1-ethoxycarbonyl)ethyl)monophosphonamido]furanyl}-thiazole (5.9). Foam. Anal. Calcd for C₂₃H₃₄N₃O₈PS: C, 50.82; H, 6.30; N, 7.73. Found: C, 50.54; H, 5.93; N, 7.68.

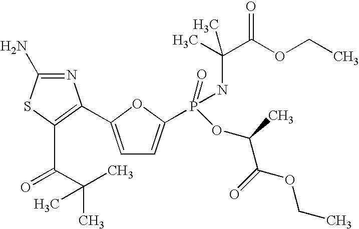

2-amino-5-(2,2-dimethylpropionyl)-4-{2-[5-(N-(1-ethoxycarbonyl-prop-2-yl)-O-(((S)-1-isopropyloxycarbonyl)ethyl)monophosphonamido]furanyl}-thiazole (5.10). Foam. Anal. Calcd for C₂₄H₃₆N₃O₈PS: C, 51.70; H, 6.51; N, 7.54. Found: C, 51.32; H, 6.17; N, 7.59.

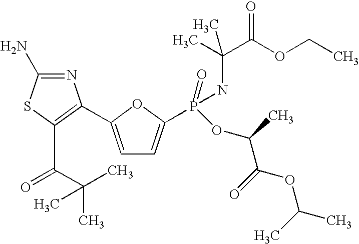

2-amino-5-(2,2-dimethylpropionyl)-4-{2-[5-(N-(ethoxycarbonyl-methyl)-O-(ethoxycarbonylmethyl)monophosphonamido]furanyl}thiazole (5.11). Foam. Anal. Calcd for C₂₀H₂₈N₃O₈PS+0.2 CH₂Cl₂: C, 46.79; H, 5.52; N, 8.10. Found: C, 46.90; H, 5.67; N, 7.83.

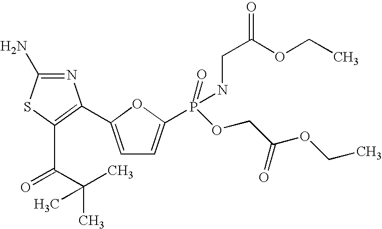

2-amino-5-(2-ethylbutyryl)-4-{2-[5-(N-(2-ethoxycarbonyl-prop-2-yl)-O-(ethoxycarbonylmethyl)monophosphonamido]furanyl}thiazole (5.12). Foam. Anal. Calcd for C₂₃H₃₄N₃O₈PS: C, 50.82; H, 6.30; N, 7.73. Found: C, 52.18; H, 6.23; N, 7.67.

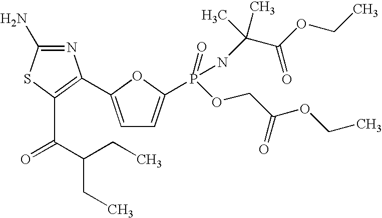

### Example 6

### Preparation of Sate Phosphonate Esters as Prodrugs

Step A. A solution of {5-[2-amino-5-(2,2-dimethyl-propionyl)thiazol-4-yl]furan-2-yl}phosphonic acid (1.1) (1 mmole) and thionyl chloride (4 mmole) in 1,2-dichloroethane was heated at 50° C. for 2 h. The reaction solution was evaporated to dryness and the residue was redissolved in 1,2-dichloroethane. After cooling to 0° C., S-acetyl-2-thioethanol (prepared according to literature procedures, 3 mmole) and N,N-diethylisopropylamine (3.5 mmole) were added. After stirring at 25° C. for 12 h, the reaction was subjected to extraction and chromatography to give 2-amino-5-(2-ethylbutyryl)-4-{[5-(O,O′-bis(S-acetyl-2-thioethyl)-phosphono]furan-2-yl}-thiazole (6.1) as a foam. Anal. Calcd for C₂₁H₂₉N₂O₇PS₃: C, 45.97; H, 5.33; N, 5.11. Found: C, 46.08; H, 5.52; N, 5.20.

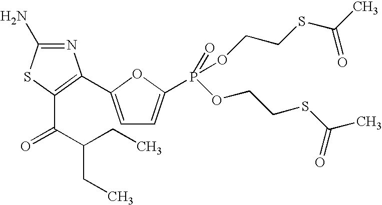

The following compounds were prepared according to the above described procedures or in some cases with minor modifications of these procedures using conventional chemistry:

2-amino-5-(2-ethylbutyryl)-4-{[5-(O,O′-bis(S-benzoyl-2-thioethyl)-phosphono]furan-2-yl}thiazole (6.2). Foam. Anal. Calcd for C₃₁H₃₃N₂O₇PS₃+0.1 MeOH: C, 55.36; H, 4.96; N, 4.35. Found: C, 55.76; H, 5.36; N, 4.51.

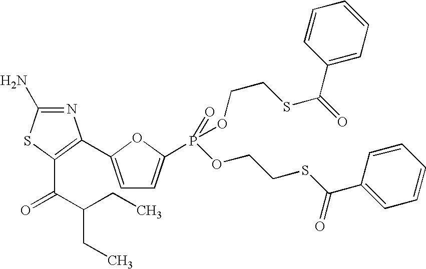

2-amino-5-cyclobutylcarbonyl-4-{[5-(O,O′-bis(S-acetyl-2-thioethyl)-phosphono]furan-2-yl}thiazole (6.3). Foam. Anal. Calcd for C₂₀H₂₅N₂O₇PS₃: C, 45.10; H, 4.73; N, 5.26. Found: C, 44.93; H, 5.08; N, 5.55.

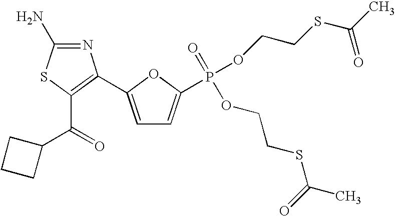

2-amino-5-(2,2-dimethyl-propionyl)-4-{[5-(O,O′-bis(S-propionyl-2-thioethyl)phosphono]furan-2-yl}thiazole (6.4). Foam. Anal. Calcd for C₂₂H₃₁N₂O₇PS₃: C, 46.96; H, 5.55; N, 4.98. Found: C, 46.86; H, 5.16; N, 5.23.

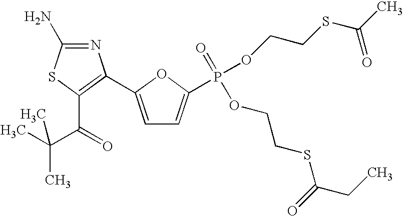

2-amino-5-(2,2-dimethyl-propionyl)-4-{[5-(O,O′-bis(S-benzoyl-2-thioethyl)phosphono]furan-2-yl}thiazole (6.5). Foam. Anal. Calcd for C₃₀H₃₁N₂O₇PS₃: C, 54.70; H, 4.74; N, 4.25. Found: C, 52.99; H, 4.89; N, 4.16.

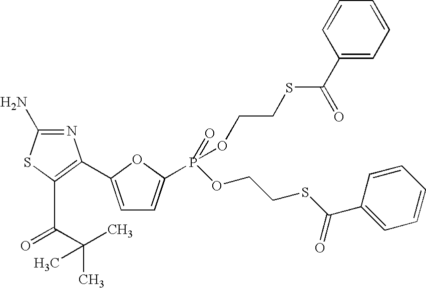

2-amino-5-(2,2-dimethyl-propionyl)-4-{[5-(O,O′-bis(S-ethoxy-carbonyl-2-thioethyl)phosphono]furan-2-yl}thiazole (6.6). Foam. Anal. Calcd for C₂₂H₃₁N₂O₉PS₃: C, 44.44; H, 5.25; N, 4.71. Found: C, 44.08; H, 5.59; N, 4.67.

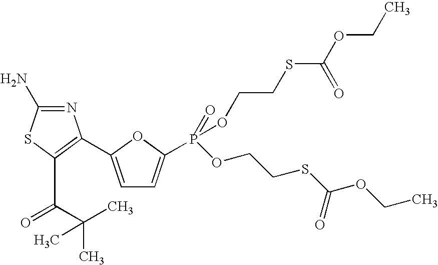

2-amino-5-(2,2-dimethyl-propionyl)-4-{[5-(O,O′-bis(S-isopropyloxy-carbonyl-2-thioethyl)phosphono]furan-2-yl}thiazole (6.7). Foam. Anal. Calcd for C₂₄H₃₅N₂O₉PS₃+0.4H₂O: C, 45.76; H, 5.73; N, 4.45. Found: C, 45.43; H, 5.88; N, 4.52.

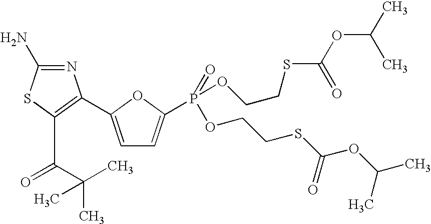

2-amino-5-(2,2-dimethyl-propionyl)-4-{[5-(O,O′-bis((2-acetylthio)-cyclohexyl)phosphono]furan-2-yl}thiazole (6.8). Foam. Anal. Calcd for C₂₈H₃₉N₂O₇PS₃: C, 52.32; H, 6.12; N, 4.36. Found: C, 51.96; H, 5.85; N, 4.48.

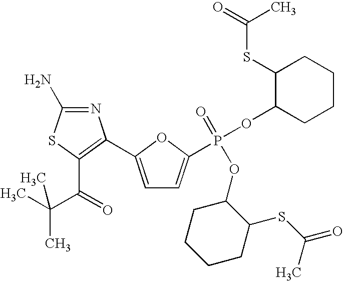

### Example 7

### Preparation of Phosphonate 1,3-propandiol Cyclic Esters as Prodrugs

Step A. A solution of {5-[2-amino-5-(2,2-dimethyl-propionyl)thiazol-4-yl]furan-2-yl}phosphonic acid (1.1) (1 mmole) and thionyl chloride (4 mmole) in 1,2-dichloroethane was heated at 50° C. for 2 h. The reaction solution was evaporated to dryness and the residue was redissolved in 1,2-dichloroethane. After cooling to 0° C., 1-(3-chlorophenyl)-1,3-propandiol (1.5 mmole) and N,N-diethylisopropylamine (3.5 mmole) were added. After stirring at 25° C. for 12 h, the reaction was subjected to extraction and chromatography to give (cis)-2-amino-5-(2,2-dimethylpropionyl)-4-{[5-(4-(3-chlorophenyl)-2-oxo-1,3,2-phosphorinan-2-yl)]furan-2-yl}thiazole (7.1) as a yellow foam. Anal. Calcd for C₂₁H₂₂N₂O₅PSCl+0.2H₂O: C, 52.06; H, 4.66; N, 5.78. Found: C, 51.67; H, 5.00; N, 5.66.

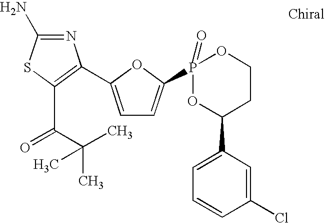

The following compounds were prepared according to the above described procedures or in some cases with minor modifications of these procedures using conventional chemistry:

(trans)-2-amino-5-(2,2-dimethylpropionyl)-4-{[5-(4-(3-chlorophenyl)-2-oxo-1,3,2-phosphorinan-2-yl)]furan-2-yl}thiazole (7.2). Foam. Anal. Calcd for C₂₁H₂₂N₂O₅PSCl+0.2H₂O: C, 52.06; H, 4.66; N, 5.78. Found: C, 51.76; H, 5.00; N, 5.41.

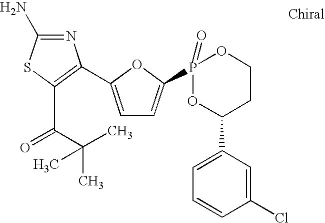

(trans)-2-amino-5-(2,2-dimethylpropionyl)-4-{[5-(4-(4-pyridyl)-2-oxo-1,3,2-phosphorinan-2-yl)]furan-2-yl}thiazole (7.3). Foam. Anal. Calcd for C₂₀H₂₂N₃O₅PS+0.45H₂O+0.15 EtOAc: C, 52.78; H, 5.18; N, 8.96. Found: C, 52.80; H, 5.16; N, 9.03.

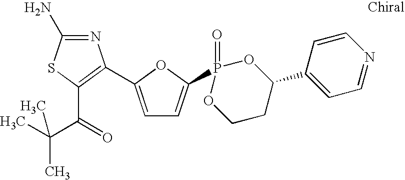

(cis)-2-amino-5-(2,2-dimethylpropionyl)-4-{[5-(4-(4-pyridyl)-2-oxo-1,3,2-phosphorinan-2-yl)]furan-2-yl}thiazole (7.4). Foam. Anal. Calcd for C₂₀H₂₂N₃O₅PS+0.8H₂O+0.1 EtOAc: C, 52.06; H, 5.23; N, 8.93. Found: C, 51.80; H, 5.13; N, 9.06.

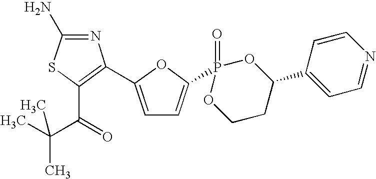

### Example 8

### Preparation of Phosphonate Acyloxyalkyl and Alkyloxycarbonyloxyalkyl Esters as Prodrugs

Step A. A mixture of {5-[2-amino-5-(2,2-dimethyl-propionyl)thiazol-4-yl]furan-2-yl}phosphonic acid (1.1) (1 mmole) and Hunig's base (N,N-diisopropylethylamine) (4 mmole) in acetonitrile was treated with POM-I (pivolate iodomethyl ester, which was prepared following literature procedures) at 25° C. for 24 h. The reaction was subjected to extraction and chromatography to give 2-amino-5-(2,2-dimethylpropionyl)-4-{[5-(O,O′-bis(pivalyloxymethyl)phosphono)]furan-2-yl}thiazole (8.4) as an off-white solid. Anal. Calcd for C₂₄H₃₅N₂O₉PS: C, 51.61; H, 6.32; N, 5.01. Found: C, 51.65; H, 6.15; N, 5.22.

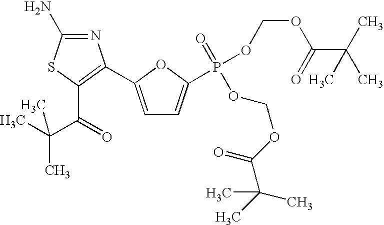

The following compounds were prepared according to the above described procedures or in some cases with minor modifications of these procedures using conventional chemistry:

2-amino-5-cyclobutylcarbonyl-4-{[5-(O,O′-bis(pivalyloxymethyl)-phosphono)]furan-2-yl}thiazole (8.1). Yellow solid. Anal. Calcd for C₂₄H₃₃N₂O₉PS: C, 51.79; H, 5.98; N, 5.03. Found: C, 51.83; H, 6.14; N, 5.03.

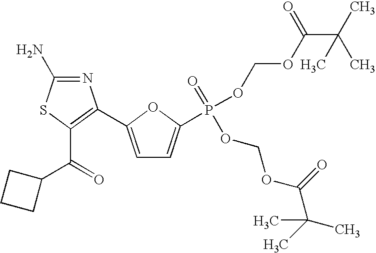

2-amino-5-(2-ethylbutyryl)-4-{[5-(O,O′-bis(pivalyloxymethyl)-phosphono)]-furan-2-yl}thiazole (8.2). Foam. Anal. Calcd for C₂₅H₃₇N₂O₉PS: C, 52.44; H, 6.51; N, 4.89. Found: C, 52.37; H, 6.55; N, 4.99.

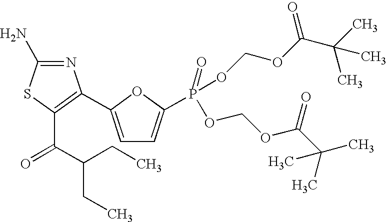

2-amino-5-(2,2-dimethylpropionyl)-4-{[5-(O,O′-bis(ethoxycarbonyl-oxymethyl)phosphono)]furan-2-yl}thiazole (8.3). Yellow oil. Anal. Calcd for C₂₀H₂₇N₂O₁₁PS 0.2 CH₂Cl₂: C, 44.00; H, 5.01; N, 5.08. Found: C, 44.09; H, 5.07; N, 5.24.

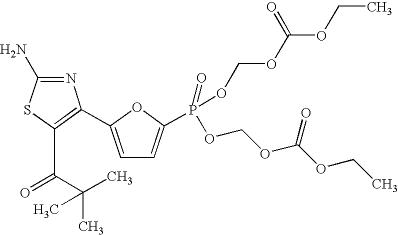

### Example 9

### Preparation of Monophosphoramides

Step A. A solution of {5-[2-amino-5-(2,2-dimethyl-propionyl)thiazol-4-yl]furan-2-yl}phosphonic acid (1.1) (1 mmole), DMF (1.1 mmole) and oxalyl chloride (3.2 mmole) in dichloromethane was heated at 50° C. for 2 h. The reaction solution was evaporated to dryness and the residue was redissolved in dichloromethane and cooled to 0° C. In another flask, a suspension of 2-methylalanine ethyl ester hydrogen chloride salt (1 mmole) in dichloromethane was treated with N,N-diethylisopropylamine (6 mmole). After 15 min, this solution was added to the initial dichloridate solution cooled at 0° C., and stirred at 25° C. for 2 h. Ethanol (10 mmole) was added and the reaction solution was stirred at 25° C. for 12 h. The reaction was subjected to extraction and chromatography to give 2-(dimethylamino-methyleneamino)-5-(2,2-dimethyl-propionyl)-4-{2-[5-(N-(2-ethoxycarbonylprop-2-yl)-O-ethyl)monophosphonamido]furanyl}thiazole.

Step B. A solution of 2-(dimethylamino-methyleneamino)-5-(2,2-dimethyl-propionyl)-4-{2-[5-(N-(2-ethoxycarbonylprop-2-yl)-O-ethyl)monophosphonamido]-furanyl}thiazole (1 mmole) in ethanol was treated with acetic acid (20 mmole) and heated to reflux for 12 h. The reaction was evaporated to dryness and the residue was subjected to extraction and chromatography to give 2-amino-5-(2,2-dimethylpropionyl)-4-{2-[5-(N-(2-ethoxycarbonylprop-2-yl)-O-ethyl)monophosphonamido]furanyl}thiazole.

Step C. A solution of 2-amino-5-(2,2-dimethyl-propionyl)-4-{2-[5-(N-(2-ethoxycarbonylprop-2-yl)-O-ethyl)monophosphonamido]furanyl}thiazole (1 mmole) in ethanol-water was treated with lithium hydroxide (20 mmole) and stirred at 25° C. for 12 h. The pH of the reaction was adjusted to 5.4 and extracted with dichloromethane. The aqueous phase was then adjusted to pH 11 and evaporated to dryness. The solid was dissolved in water, filtered and the filtrate was diluted with acetone to give a yellow solid. The solid was collected via filtration and dried to give 2-amino-5-(2,2-dimethylpropionyl)-4-{2-[5-(N-(2-carboxylprop-2-yl)monophosphonamido]furanyl}thiazole dilithium salt (9.1). Pale yellow powder. Anal. Calcd for C₁₆H₂₀N₃O₆PSLi₂+3H₂O: C, 39.93; H, 5.44; N, 8.73; Li: 2.88. Found: C, 39.91; H, 5.08; N, 8.52; Li: 3.03.

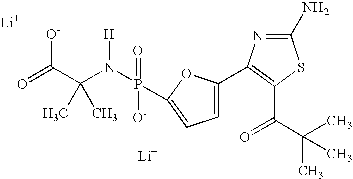

### Example 10

### Preparation of Monophosphoramides

Step A. A solution of {5-[2-amino-5-(2,2-dimethyl-propionyl)thiazol-4-yl]furan-2-yl}phosphonic acid (1.1) (1 mmole), DMF (1.1 mmole) and oxalyl chloride (3.2 mmole) in dichloromethane was heated at 50° C. for 2 h. The reaction solution was evaporated to dryness and the residue was redissolved in dichloromethane and cooled to 0° C. In another flask, a suspension of 2-methylalanine ethyl ester hydrogen chloride salt (1 mmole) in dichloromethane was treated with N,N-diethylisopropylamine (6 mmole). After 15 min, this solution was added to the initial dichloridate solution cooled at 0° C., and stirred at 25° C. for 2 h. Benzyl alcohol (2 mmole) was added and the reaction solution was stirred at 25° C. for 12 h. The reaction was subjected to extraction and chromatography to give 2-(dimethylamino-methyleneamino)-5-(2,2-dimethyl-propionyl)-4-{2-[5-(N-(2-ethoxycarbonyl-prop-2-yl)-O-benzyl)monophosphonamido]furanyl}thiazole.

Step B. A solution of 2-(dimethylamino-methyleneamino)-5-(2,2-dimethyl-propionyl)-4-{2-[5-(N-(2-ethoxycarbonylprop-2-yl)-O-benzyl)monophosphonamido]-furanyl}thiazole (1 mmole) in ethanol was treated with acetic acid (20 mmole) and heated to reflux for 12 h. The reaction was evaporated to dryness and the residue was subjected to extraction and chromatography to give 2-amino-5-(2,2-dimethylpropionyl)-4-{2-[5-(N-(2-ethoxycarbonylprop-2-yl)-O-benzyl)monophosphonamido]furanyl}-thiazole.

Step C. A solution of 2-amino-5-(2,2-dimethyl-propionyl)-4-{2-[5-(N-(2-ethoxycarbonylprop-2-yl)-O-benzyl)monophosphonamido]furanyl}thiazole (0.057 mmole) and triethylamide (0.17 mmole) in ethanol was treated with palladium on carbon (10%) (6 mg) and stirred at 25° C. under 1 atmosphere of hydrogen for 12 h. The reaction mixture was filtered through a celite pad and the filtrate was evaporated to give a 2-amino-5-(2,2-dimethylpropionyl)-4-{2-[5-(N-(2-ethoxycarbonylprop-2-yl)mono-phosphonamido]-furanyl}thiazole triethylamine salt (10.1) as a yellow foam. Pale yellow powder. Anal. Calcd for C₁₈H₂₆N₃O₆PS+1.3H₂O+1 TEA: C, 50.74; H, 7.74; N, 9.86. Found: C, 50.81; H, 7.85; N, 9.64.

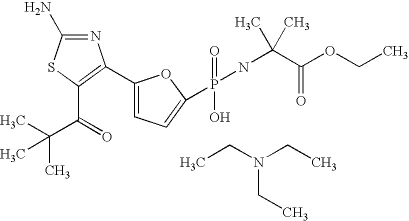

In a similar manner, 2-amino-5-(2,2-dimethyl-propionyl)-4-{2-[5-(N-(2-benzyloxycarbonylprop-2-yl)-O-benzyl)monophosphonamido]furanyl}-thiazole was prepared by using 2-methylalanine benzyl ester hydrogen chloride salt for step A, and following steps B and C 2-amino-5-(2,2-dimethylpropionyl)-4-{2-[5-(N-(2-carboxylprop-2-yl)monophosphonamido]furanyl}thiazole triethylamine salt (10.2) as a yellow foam.

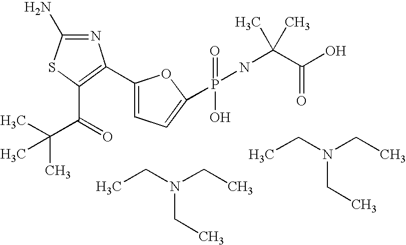

### Example 11

### Preparation of Salt Forms of Bisamidates

Step A. A solution of 2-amino-5-(2,2-dimethylpropionyl)-4-{[5-(N,N′-(2-ethoxycarbonylprop-2-yl)phosphonamido]furan-2-yl}thiazole (2.1) (1 mmole) in ethanol was treated with methanesulfonic acid (1.1 mmole) at 25° C. for 1 h. The reaction solution was evaporated to dryness and the residue was treated with acetone to give a precipitate, which was collected via filtration and dried to give 2-amino-5-(2,2-dimethylpropionyl)-4-{[5-(N,N′-(2-ethoxycarbonylprop-2-yl)-phosphonamido]furan-2-yl}thiazole methanesulfonic acid salt (11.1) as a white solid. Anal. Calcd for C₂₄H₃₇N₄O₇PS+C₁H₄O₃S+0.1C₃H₆O: C, 46.14; H, 6.37; N, 8.51; S: 9.74. Found: C, 46.84; H, 6.41; N, 8.54; S: 9.99.

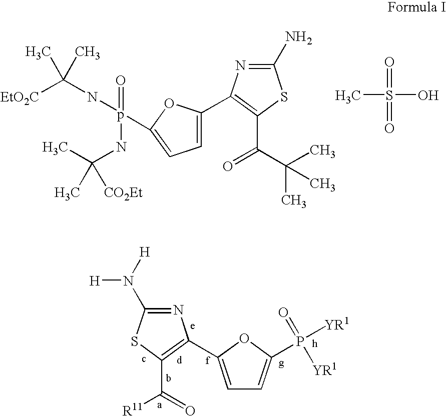

Compounds of Formula I can be derived from formation of bonds a-g in any order. For example, in Schemes 1 and 3, the key bond forming steps, in order are: d, g, c/e, h, wherein the bonds a, b and f are present in the commercially available starting materials. In Scheme 4, bond f is formed last, whereas in Scheme 5, bond g is formed last. Scheme 6 involves early formation of bond g, whereas in Scheme 7, bond e is formed followed by bond d, using a Mannich reaction.

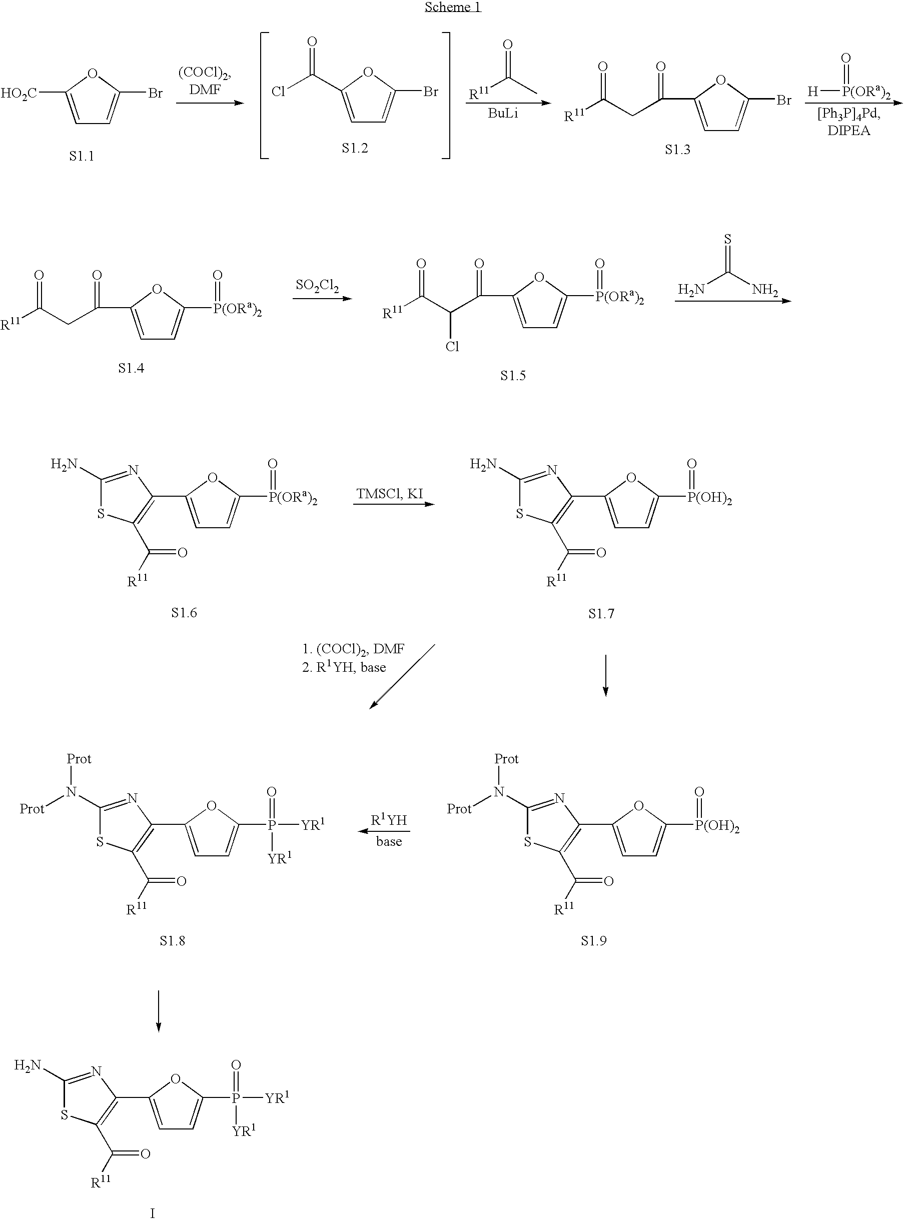

Compounds of Formula I can be prepared by the synthetic scheme shown in Scheme 1. 5-Bromo-2-furoic acid S1.1 is converted to the acid chloride S1.2 with oxalyl chloride or other suitable reagent. The acid chloride S1.2 is condensed with the anion of a methyl, R¹¹ ketone, where R¹¹ is alkyl, aryl or a heterocyclic group, to form diketone S1.3. The bromofuran diketone S1.3 is phosphonylated with dialkylphosphite or diarylphosphite to the diketone S1.4 using a suitable transition metal catalyst complex such as tetrakis(triphenylphosphine)palladium(0). The diketone S1.4 is halogenated with a suitable reagent such as bromine or sulfuryl chloride to provide crude halodiketone S1.5 as a thick oil. The halodiketone S1.5 is condensed with thiourea to provide thiazole S1.6. The dialkylphosphonate or diarylphosphonate functionality of S1.6 is deprotected a using suitable reagent such as a trimethylsilyl halide, sodium hydroxide or mineral acid in an alcohol to provide the phosphonic acid S1.7.

In one synthetic pathway, the phosphonic acid S1.7 is converted to an amidine-protected phosphonodichloridate using suitable reagents such as oxalyl chloride with a dialkylformamide, or thionyl chloride. The phosphonodichoridate is treated with a suitable primary or secondary amine and a suitable acid-scavenging base such as triethylamine or diisopropylethylamine (DIPEA), to provide the crude bis-amidate S1.8 (Prot-N(Prot)- is R—N(R′)—C(H)═N— wherein R and R′ are independently C₁₋₄ alkyl). The amidine protecting group is removed with a suitable reagent such as acetic acid in ethanol to form the product I.

The synthetic pathway described in the previous paragraph illustrates the formation of S1.8 from S1.7 in which the same reagent(s)—oxalyl chloride/dimethylformamide—serves both to activate the phosphonic ester moiety and to protect the exocyclic amino group of the compound of Formula S1.7 as an amidine, i.e., the activation of the phosphonic acid moiety of S1.7 and the protection of the exocyclic amino moiety of S1.7 are concurrent. This pathway is not favored when in the desired compound of Formula I, the moiety —YR¹ is of the acyloxyalkyl type. However, in another synthetic pathway illustrated in Scheme 1, the exocyclic nitrogen of S1.7 is first protected with a suitable amino-protecting group to form phosphonic acid S1.9. The phosphonic acid S1.9 is then activated, and treated as described for the phosphonodichoridate in the preceding paragraph. The protecting group is removed with a suitable reagent to form the product I. This pathway is favored for making compounds of Formula I wherein —YR¹ is of the acyloxyalkyl type, but is also suitable for the entire scope of —YR¹ as that moiety is defined above.

In another aspect, the phosphonic acid S1.7 can be directly transformed to a compound of Formula I. In this aspect, the phosphonic acid S1.7 is activated as described above, and then treated with a suitable primary or secondary amine and a suitable acid-scavenging base as described above.

More generally, compounds of Formula I can be prepared by the following method. A compound of Formula C1.1:

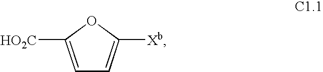

wherein X^{b} is halo,

is converted into a compound of Formula C1.2:

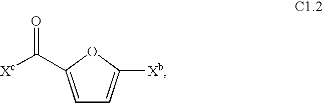

wherein X^{c} is halo.

Useful values of X^{b} include F, Cl, Br and I. More useful values of X^{b} include I and Br, particularly Br.

Useful values of X^{c} include F, Cl, Br and I. More useful values of X^{c} include Cl and Br, particularly Cl.

Reagents useful for effecting this conversion are known in the art (see, e.g., R. C. Larock, Comprehensive Organic Transformations, 2d ed., John Wiley & Sons: New York (1999)) and include oxalyl chloride, thionyl chloride, POCl₃, PCl₃, PCl₅, oxalyl bromide, thionyl bromide, PBr₃, PBr₅, BBr₃—Al₂O₃, SeF₄/pyridine, I₂/H₂SiI₂ and the like. The reaction can be carried out in a suitable solvent, such as DMF, carbon tetrachloride, chloroform and the like, at a suitable temperature, such as from 0° C. to about 80° C.

A compound of formula R¹¹—C(O)—CH₃, wherein R¹¹ is defined as above, is deprotonated to form an anion, and the anion is reacted with the compound of Formula C1.2 to form a compound of Formula C1.3:

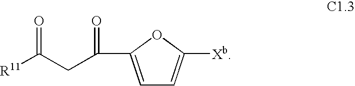

Bases useful for the deprotonation are known in the art and include n-butyllithium, t-butyllithium, potassium tert-butoxide, sodium bis(trimethylsilyl)amide, lithium diisopropylamide (LDA) and the like. The deprotonation can be carried out in a suitable solvent, such as tetrahydrofuran (THF), dimethylsulfoxide (DMSO), dimethylformamide (DMF), dimethylacetamide (DMA) and the like, at a suitable temperature, such as from about 0° C. to about −78° C.

The compound of Formula C1.3 is phosphonylated with a compound of formula H—P(O)(OR^{a})₂, wherein R^{a} is C₁₋₄ alkyl, to form a compound of Formula C1.4:

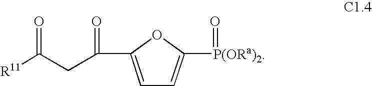

Useful values of R^{a} include methyl, ethyl, propyl, isopropyl, butyl, isobutyl, s-butyl and t-butyl. More useful values of R^{a} include methyl, ethyl, isopropyl and t-butyl.

The phosphonylation is carried out, e.g., with a transition metal catalyst in the presence of a base.

Transition metal catalysts useful for this phosphonylation include palladium catalysts such as [Ph₃P]₄Pd, Cl₂[Ph₃P]₂Pd, Pd(OAc)₂/P(OiPr)₃, Pd₂(dba)₃/BINAP and the like.

Bases useful in this phosphonylation include non-nucleophilic amine bases such as diisopropylethylamine, triethylamine, dimethylaminopyridine and the like, and inorganic bases such as sodium bicarbonate, potassium carbonate.

The compound of Formula C1.4 is halogenated to form a compound of Formula C1.5:

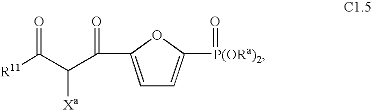

wherein X^{a} is halo.

Useful values of X^{a} include F, Cl, Br and I. More useful values of X^{a} include Cl and Br, particularly Cl.

Reagents useful for effecting the halogenation are known in the art and include sulfuryl chloride, thionyl chloride, thionyl bromide, LDA/(PhSO₂)₂NF, base/CH₃CO₂F, base/I₂, bromine/base and the like. [The reaction can be carried out in a suitable solvent, such as dichloromethane, carbon tetrachloride, chloroform, DMF and the like, at a suitable temperature, such as from 0° C. to about 80° C.

The compound of Formula C1.5 is reacted with thiourea to form a compound of Formula C1.6:

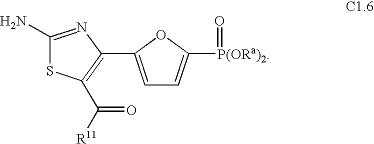

The reaction can be carried out in a suitable solvent, such as ethyl acetate, isopropanol, ethanol and the like, at a suitable temperature, such as from about 0° C. to about 90° C.

The compound of Formula C1.6 is deprotected to form a compound of Formula C1.7:

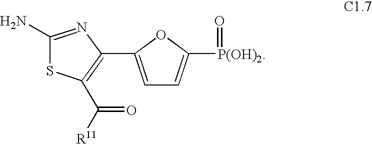

Reagents useful for deprotecting compounds of Formula C1.6 are known in the art and include TMSCl/KI, TMSBr/KI or TMSI/KI, followed by mild hydrolysis of the resulting silyl phosphonate ester; HCl; HBr; forming the dichlorophosphonate via a halogenating agent such as PCl₅, SOCl₂, etc. followed by aqueous hydrolysis; hydrolysis in the presence of acid such as HBr and HBr—AcOH; and base promoted hydrolysis such as sodium hydroxide or potassium hydroxide in ethylene glycol at the appropriate temperature. The deprotection reaction can be carried out in a suitable solvent, such as acetonitrile, methylene chloride, chloroform and the like, at a suitable temperature, such as from about 20° C. to about 200° C.

The compound of Formula C1.7 is activated, and the activated compound of Formula C1.7 is reacted with a compound of formula R¹YH, wherein R¹ and Y are defined as above, in the presence of an acid scavenger, to form a compound of Formula C1.8: 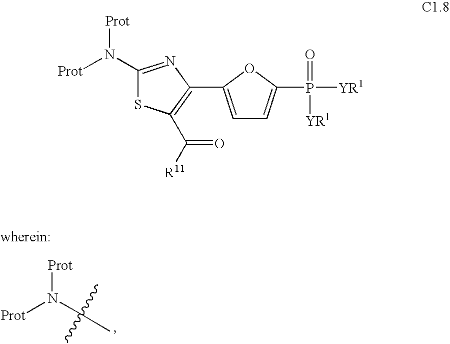
is a protected amino group,

wherein, as discussed above, the exocyclic amino group of the compound of Formula C1.7 is protected before the activation or concurrently with the activation. In the former case, a compound of Formula C1.9:

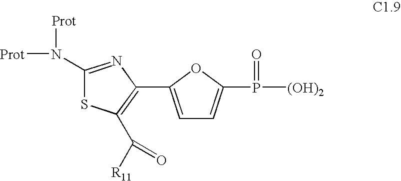

is formed. This compound is then activated and reacted as described above.

The moiety Prot-N(Prot)- is an amino group protected with any group suitable for protecting amines. Examples of useful protecting groups, their formation and their removal are found in Protective groups in Organic Synthesis, Greene, T. W., 1991, Wiley, New York, which is incorporated by reference herein in its entirety, and include. Useful protecting groups include carbamates such as Boc and Cbz, and dialkyl amidines, i.e., where Prot-N(Prot)- is R—N(R′)—C(H)═N— wherein R and R′ are independently C₁₋₄ alkyl.

By “activating” the compound of Formula C1.7 is meant transforming it into a compound that will react with a compound of formula R¹YH to form a compound of Formula C1.8. Suitable methods of activating phosphonic acids are known in the art and include, e.g., converting the compound of Formula C1.7 into its corresponding phosphonodichloridate using, e.g., oxalyl chloride/dialkylformamide, thionyl chloride, thionyl chloride/dialkylformamide and phosphoryl chloride. Activation with, e.g., oxalyl chloride/dialkylformamide can be carried out in a suitable solvent, such as dichloromethane, 1,2-dichloroethane, chloroform and the like, at a suitable temperature, such as from about 25° C. to about 70° C.

The reaction of a compound of formula R¹YH to form a compound of Formula C1.8 can be carried out in a suitable solvent, such as dichloromethane, 1,2-dichloroethane, chloroform, acetonitrile, DMF, THF and the like, at a suitable temperature, such as from about −20° C. to about 60° C.

Suitable acid scavengers are known in the art and include non-nucleophilic bases such as triethylamine, diisopropylethylamine, dimethylaminopyridine, tetramethylethylenediamine, 2,6-lutidine and the like.

The compound of Formula C1.8 is deprotected to form the compound of Formula I. The deprotection can be carried out under suitable deprotection conditions, such as with acetic acid in isopropanol and the like, at a suitable temperature, such as from about 25° C. to about 100° C.

Alternatively, the compound of Formula I can be formed directly from a compound of Formula C1.7 without protecting the exocyclic amino moiety. The compound of Formula C1.7 is activated as described above, then treated with a compound of formula R¹YH, wherein R¹ and Y are defined as above, in the presence of an acid scavenger, as described above.

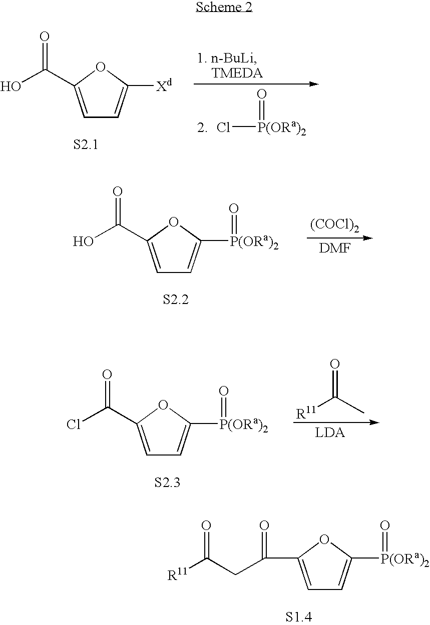

An alternate approach to prepare diketone S1.4 of Scheme 1 is shown in Scheme 2. Starting from 2-furoic acid or 5-bromo-2-furoic acid the phosphonate functionality is incorporated through a metalation at the 5-position with a suitable base such as butyllithium and a suitable complexing agent such as tetramethylethylenediamine, and subsequent addition to a phosphate ester or halide to form carboxylic acid S2.2. The carboxylic acid S2.2 is converted to the acid chloride S2.3 with a suitable reagent such as oxalyl chloride. The acid chloride S2.3 is condensed with the anion of a methyl, R¹¹ ketone, where R¹¹ is alkyl, aryl or a heterocyclic group, to form diketone S1.4.

More generally, compounds of Formula C1.4 can be prepared by the following method. A compound of Formula C2.1:

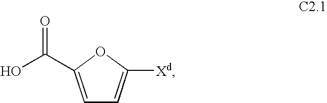

(1) wherein X^{d} is hydrogen, is reacted with a base, or (2) wherein X^{d} is halo, is reacted with a metalizing agent, to form a dianion, and the dianion is reacted with a compound of formula X′—P(O)(OR^{a})₂, wherein R^{a} is defined as above, and X′ is halo or —OR′ wherein R′ is C₁₋₄ alkyl or —P(O)(OR^{a})₂, to form a compound of Formula C2.2:

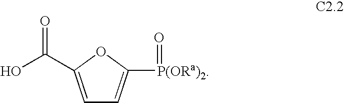

Useful values of X^{d} include H, F, Cl, Br and I. More useful values of X^{d} include H, I and Br, particularly Br.

When X′ is halo, useful values of X′ include F, Cl, Br and I. More useful values of X′ include Cl and Br, particularly Cl.

When X′ is —OR′, useful values of R′ include methyl, ethyl, propyl, isopropyl, butyl, isobutyl, s-butyl and t-butyl. When X′ is —OR′, more useful values of R′ include methyl, ethyl, isopropyl and t-butyl.

Bases and metalating agents useful in forming the dianion are known in the art and include n-butyllithium, t-butyllithium, lithiumdiisopropylamide (LDA) and the like.

The reaction of a compound of Formula C2.1 with a base or metalating agent can be carried out in a suitable solvent, such as dimethylsulfoxide (DMSO), THF, dimethylformamide (DMF), dimethylacetamide (DMA) and the like, at a suitable temperature, such as from about −78° C. to about 0° C. This reaction is optionally carried out in the presence of a complexing agent such as TMEDA.

The compound of Formula C2.2 is converted into a compound of Formula C2.3:

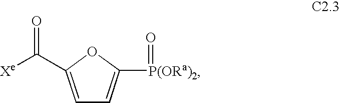

wherein X^{e} is halo.

Useful values of X^{e} include F, Cl, Br and I. More useful values of X^{e} include Cl and Br, particularly Cl.

Reagents useful for effecting this conversion are known in the art and include oxalyl chloride, oxalyl chloride/DMF, thionyl chloride, PCl₃, PCl₅, oxalyl bromide, thionyl bromide, PBr₃, PBr₅, BBr₃—Al₂O₃, SeF₄/pyridine, I₂/H₂SiI₂ and the like. The reaction can be carried out in a suitable solvent, such as dichloromethane, DMF, carbon tetrachloride, chloroform and the like, at a suitable temperature, such as from about 20° C. to about 80° C.

A compound of formula R¹¹—C(O)—CH₃, wherein R¹¹ is defined as above, is deprotonated to form an anion, and the anion is reacted with the compound of Formula C2.3.

Bases useful for the deprotonation are known in the art and include lithium diisopropylamide (LDA), n-butyl lithium, potassium tert-butoxide and the like. The deprotonation can be carried out in a suitable solvent, such as THF, dimethylsulfoxide (DMSO), dimethylformamide (DMF), dimethylacetamide (DMA) and the like, at a suitable temperature, such as from about −78° C. to about 0° C.

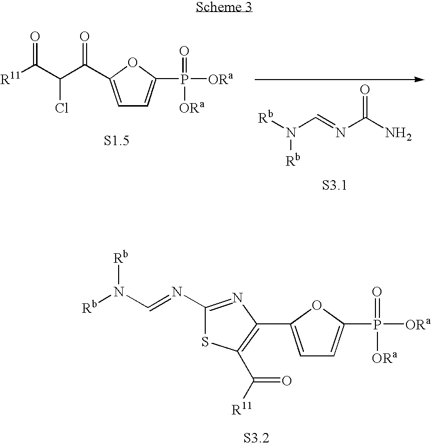

As shown in Scheme 3, cyclization of intermediate S1.5 (see Scheme 1) to thiazole S3.2 using a monofunctionalized thiourea S3.1 (e.g., R^{b} is alkyl) is envisioned where a higher ratio of the desired regioisomer to the undesired regioisomer by-product could be effected, and/or the resulting thiazole is isolated where the exocyclic nitrogen is protected. Thiazole S3.2 can then be deprotected to form a compound of Formula S1.9 and carried on to the compound of Formula I as discussed above for Scheme 1.

More generally, compounds of Formula C1.9 can be made as follows. A compound of Formula C1.5:

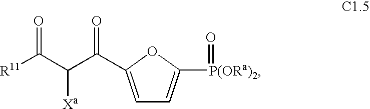

is condensed with a compound of C3.1:

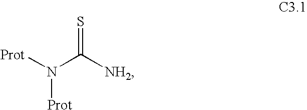

wherein R¹¹, X^{a} and R^{a} are defined as above for compounds of Formula C1.5, and 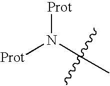
is a protected amino group,

to form a compound of Formula C3.2:

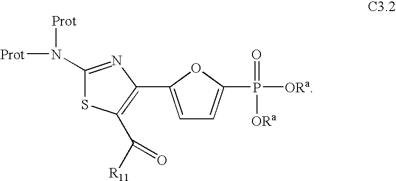

The reaction can be carried out in a suitable solvent, such as THF, ethyl acetate, ethanol, isopropanol and the like, at a suitable temperature, such as from about 0° C. to about 90° C.

Protecting groups useful for protection of the amino moiety of thiourea are known in the art and include dialkylformamidines, particularly di(C₁₋₄)alkylformamidines and the like.

The phosphate ester of Formula C3.2 is then deprotected to form a compound of

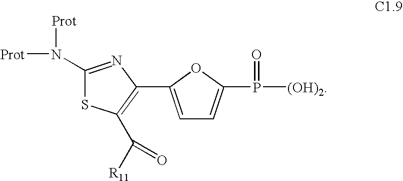

Reagents useful for deprotecting the phosphate ester of Formula C3.2 are known in the art and include those discussed above in connection with the deprotection of compounds of Formula C1.6.

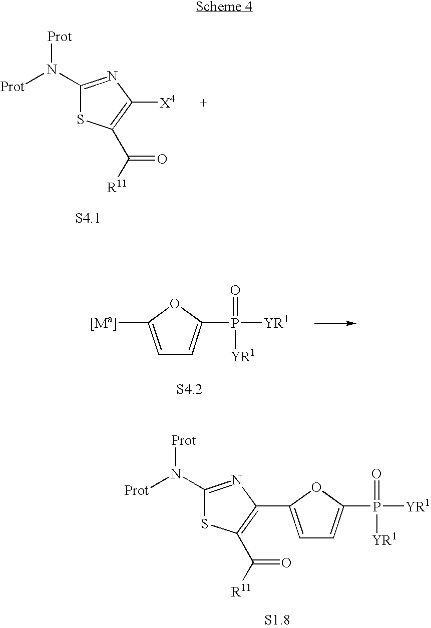

Convergent routes to compounds of Formula I are envisioned that will proceed through the thiazole-furan bond formation of suitably activated thiazole and furan components as shown in Scheme 4. The 2-furanphosphonate (Y is O) or bis-amidate (Y is NH)S4.2, suitably activated as, e.g., a boronic acid (Ma is B(OH)₂) or a metalated species (M is lithium, zinc, trialkyltin, or the like), may be coupled to a 4-halothiazole S4.1, where the exocyclic nitrogen is protected or unprotected (—N(Prot)₂ is —NH₂ or a protected amino group).

More generally, compounds of Formula C1.8 can be prepared as follows. A compound of Formula C4.1:

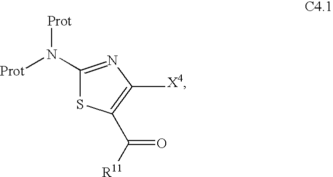

wherein R¹¹ is defined as above, X⁴ is halo, alkylsulfonyloxy or arylsulfonyloxy, and 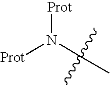
is a protected amino group,

is coupled to a compound of Formula C4.2:

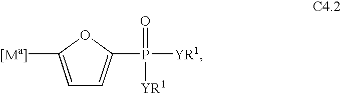

wherein Y and R¹ are defined as above, and M^{a} is —B(OH)₂, lithium, zinc, palladium, nickel or trialkyltin.

When M^{a} is palladium or nickel, the palladium or nickel atoms are suitably coordinated with ligands. Ligands suitable for use in this coupling are known in the art and include ligands such as PPh₃, dba (dibenzylidene acetone), BINAP, P(O-iPr)₃ (triisopropylphosphite), P(t-Bu)₃ and the like.

When X⁴ is halo, useful values of X⁴ include F, Cl, Br and I. More useful values of X⁴ include Cl and Br, particularly Cl.

When X⁴ is alkylsulfonyloxy or arylsulfonyloxy, useful values of X⁴ include methanesulfonyloxy, trifluoromethanesulfonyloxy and p-toluenesulfonyloxy.

The reaction can be carried out in a suitable solvent, such as dimethylsulfoxide (DMSO), dimethylformamide (DMF), dimethylacetamide (DMA), at a suitable temperature, such as from about −50° C. to about −78° C. (e.g., when Ma is lithium), or from about −25° C. to about 20° C. (e.g., when Ma is palladium).

The compound of Formula C1.8 is carried on to the compound of Formula I as discussed above for Scheme 1. Alternatively, the coupling can be carried out wherein the exocyclic nitrogen of the thiazole moiety is unprotected, i.e., a compound of Formula C4.1 in which −Prot is hydrogen. This coupling results in the formation of the compound of Formula I.

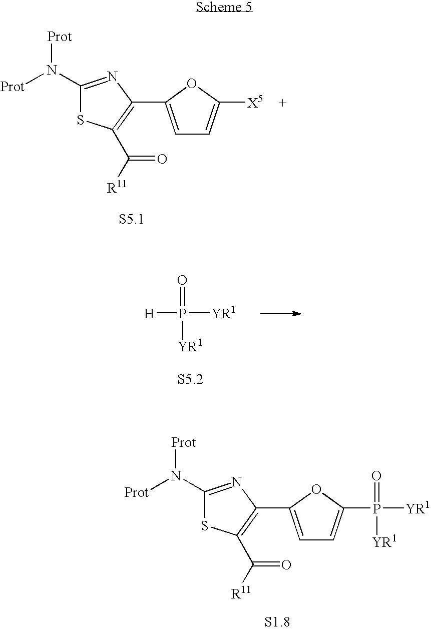

Convergent routes to compounds of Formula I are envisioned that will proceed through the furan-phosphorus bond formation as shown in Scheme 5. A suitable 2-halofuran-5-(4-thiazole) S5.1 may be coupled to a phosphonoamidite (Y is NH) or phosphite (Y is O) S5.2 via a transition metal-catalyzed coupling.

More generally, compounds of Formula C1.8 can be prepared as follows. A compound of Formula C5.1: 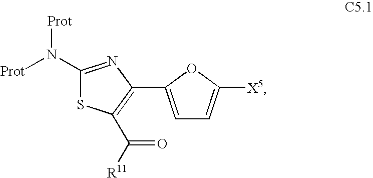
wherein R¹¹ is defined as above, X⁵ is halo and 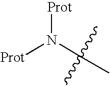
is a protected amino group,

is coupled, as described above, to a compound of formula C5.2:

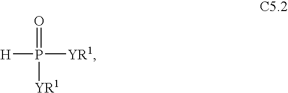

wherein Y and R¹ are defined as above.

Useful values of X⁵ include F, Cl, Br and I. More useful values of X⁵ include Cl, Br and I, particularly Cl and Br.

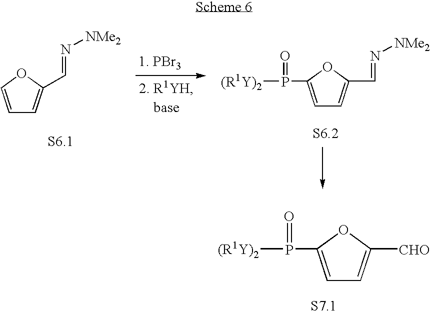

Schemes 6 and 7 together illustrate a pathway to compounds of Formula I in which bond g is formed early in the synthesis. After this bond formation, furanaldehyde is deprotected and used in a Mannich reaction as shown in Scheme 7.

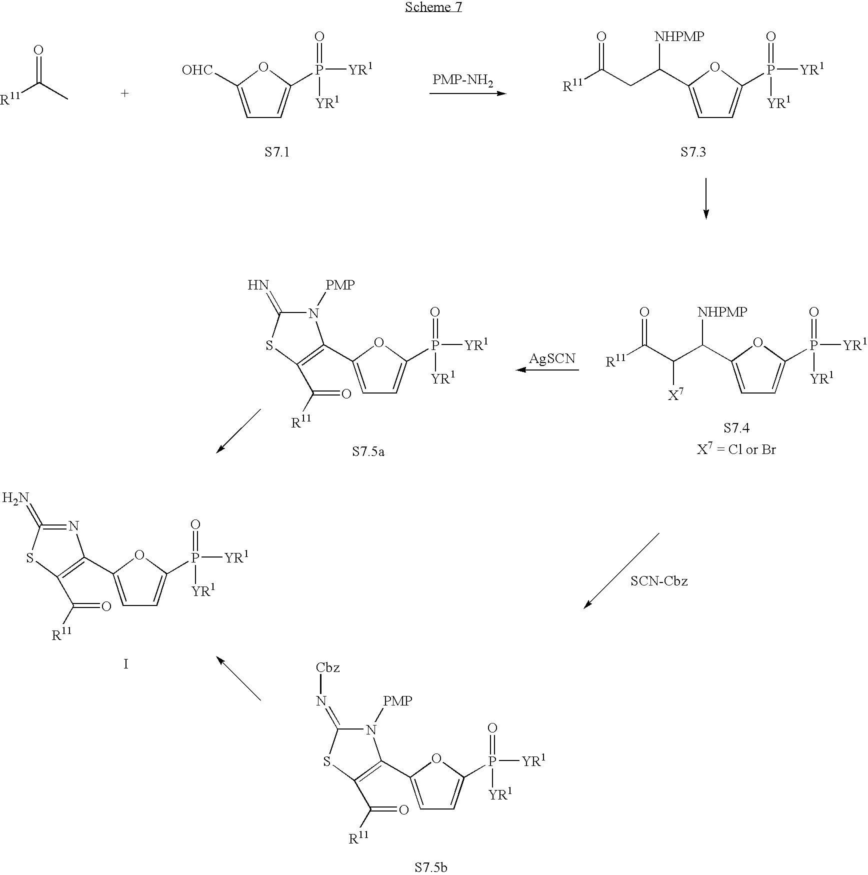

In Scheme 7, the phosphonylated furanaldehyde S7.1 undergoes a Mannich reaction with the methyl, R¹¹ ketone and a suitable nitrogen source such as para-methoxyaniline to form S7.3. After halogenation to form S7.4, the compound of Formula I can be obtained either by reaction with a suitably protected (such as with Cbz) isothiocyanate to form S7.5b followed by deprotection to complete the thiazole ring, or by reaction with a suitable thiocyanate (such as AgSCN) to form S7.5a followed by deprotection.

More generally, compounds of Formula I can be prepared by the following steps. A compound of Formula C6.1:

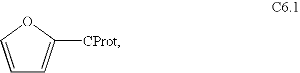

wherein CProt is a suitably protected aldehyde, is phosphonylated to form a compound of Formula C6.2:

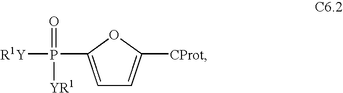

wherein Y and R¹ are defined as above.

Methods of phosphonylating are known in the art and include treatment with PBr₃ followed by R¹YH and base, or anion formation using a suitable base such as n-butyl lithium followed by reaction with an activated phosphorus compound such as Cl—PO(YR¹)₂.

These reactions can be carried out in suitable solvents such as methylene chloride, chloroform, THF and the like, at suitable temperatures such as from −78° C. to 60° C.

Examples of useful protecting groups for aldehydes, their formation and their removal may be found in Greene, supra, and include hydrazones, acetals and aminals. The compound of Formula C6.2 is deprotected to form a compound of Formula C7.1.

The compound of Formula C7.1, a compound of formula R¹¹—C(O)—CH₃, wherein R¹¹ is defined as above, and ammonia and/or an ammonium salt are condensed in a Mannich reaction. The amino group of the resulting product is protected to form a compound of Formula C7.3:

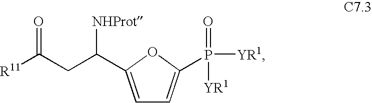

wherein Prot″ is a protecting group.

Conditions for carrying out Mannich reactions are known in the art. Suitable solvents therefor include aqueous ethanol and DMSO, and suitable acids such as HCl, sulfonic acid and proline with temperatures ranging from about 0° C. to about 100° C. Ammonium salts useful for this reaction include salts of p-methoxyaniline.

The compound of Formula C7.3 is converted into a compound of Formula C7.4:

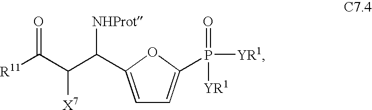

wherein X⁷ is halo.

Useful values of X⁷ include F, Cl, Br and I, particularly Cl and Br.

Reagents useful for effecting this conversion are known in the art and include sulfuryl chloride and Br₂. The conversion can be carried out in a suitable solvent such as CH₂Cl₂, CHCl₃, THF and the like, at a suitable temperature such as from about 0° C. to about 60° C.

The compound of Formula C7.4 is reacted with a compound of formula SCN-Prot′, wherein Prot′ is a protecting group, to form a compound of Formula C7.5:

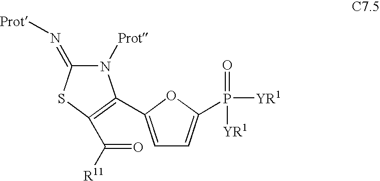

The reaction can be carried out in a suitable solvent such as include ethanol, isopropanol, CH₃CN, THF, DMF and the like, at a suitable temperature such as from about 25° C. to about 100° C.

The compound of Formula C7.5 is deprotected to form the compound of Formula I.

Throughout, each of N-Prot′ and N-Prot″ is independently a nitrogen atom protected with any group suitable for protecting the nitrogen atom of the particular functional group. Examples of useful protecting groups (such as Boc and Cbz), their formation and their removal (with reagents such as TFA, HCl, H₂ and H₂/Pd—C) may be found in Greene, supra. More useful protecting groups include carbamates such as Boc and Cbz. Also useful are protecting groups such as para-methoxyphenyl.

Alternatively, the compound of Formula C7.4 is reacted with a compound of formula M^{e}SCN, wherein M^{e} is a monocation, to form a compound of Formula C7.5 wherein Prot′ is hydrogen. The reaction can be carried out in a suitable solvent such as ethanol, isopropanol, CH₃CN, THF, DMF and the like, at a suitable reaction temperature such as from about 25° C. to about 100° C. The compound of Formula C7.5 is deprotected to form the compound of Formula I.

Useful values of M^{e} include monocations such as Ag⁺, K⁺ and Na⁺. More useful values of M^{e} include Ag⁺.

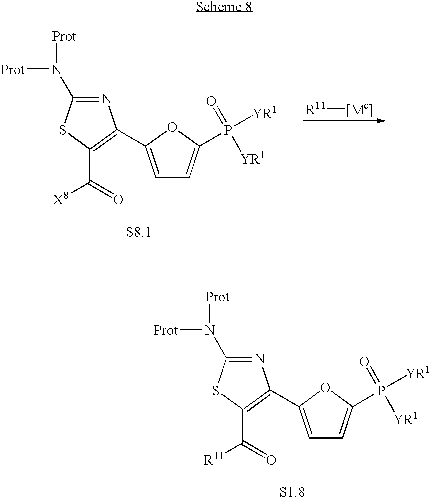

An approach involving formation of bond a last is pictured in Scheme 8. In this case, X⁸ is a suitable leaving group such as a halide or methoxy(methyl)amide, and M^{c} is a metal such as Li or Mg.

More generally, compounds of Formula C1.8 can be prepared by the following steps. A compound of formula C8.1:

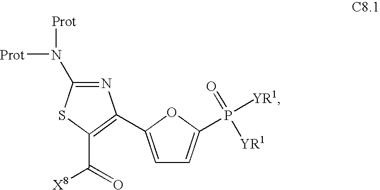

wherein Y and R¹ are defined as above, X⁸ is a leaving group, and 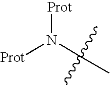
is a protected amino group,

is reacted with a compound of formula R¹¹-[M^{c}], wherein R¹¹ is defined as above, and M^{c} is a metal selected from the group consisting of lithium, magnesium and copper.

Useful values of X⁸ include F, Cl, Br and I, particularly Cl and Br; —N(Me)-OMe; and C₁₋₄ alkoxy, particularly methoxy and ethoxy.

Useful values of M^{c} include lithium, magnesium, zinc and copper, particularly lithium and magnesium. Where M^{c} is magnesium, the magnesium atom will be divalent, i.e., M^{c} will be in the form of, e.g., MgCl or MgBr. Where M^{c} is copper, the reactant is CuR¹¹—X(ligand) or CuR¹¹ (CuI). Ligands suitable for this reaction are known in the art.

The reaction can be carried out in a suitable solvent, such as THF, ethanol, dioxane, DME, toluene and the like, at a suitable temperature, such as from about 0° C. to about −78° C.

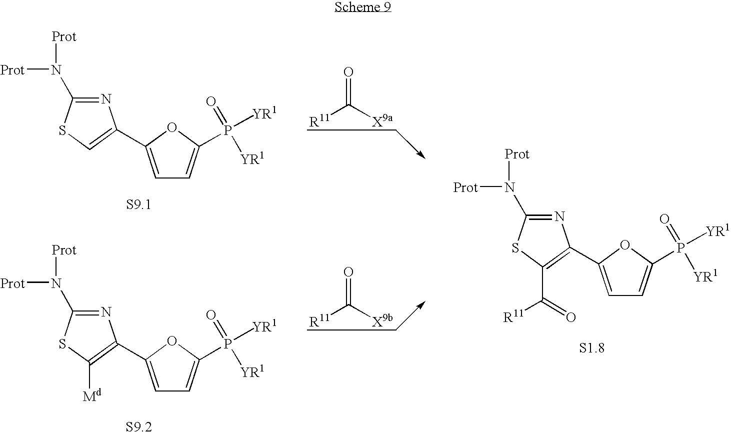

An approach involving formation of bond b last is shown in Scheme 9. In this case, the group R¹¹—C(O)— may be introduced by a Friedel-Crafts-type reaction with the electron-rich thiazole. Alternatively, a metalated version of the thiazole (e.g., M=Li) could be reacted with a suitable acylating agent R¹¹—C(O)—X.

More generally, compounds of Formula C1.8 can be prepared by the following steps. A compound of Formula C9.1:

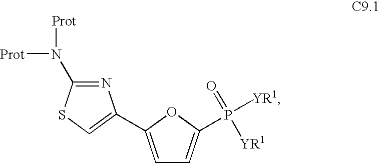

wherein Y and R¹ are defined as above, and 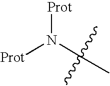
is a protected amino group,

is acylated with a compound of formula R¹¹—C(O)—X^{9a}, wherein R¹¹ is defined as above, and X^{9a} is halo, —O—C(O)—R¹¹, or alkylsulfonyloxy or arylsulfonyloxy.

When X^{9a} is halo, useful values of X^{9a} include F, Cl, Br and I, particularly Cl and Br.

When X^{9a} is alkylsulfonyloxy or arylsulfonyloxy, useful values of X^{9a} include methanesulfonyloxy, trifluoromethanesulfonyloxy and p-toluenesulfonyloxy.

More useful values of X^{9a} include halo.

The reaction can be carried out in a suitable solvent, such as methylene chloride, chloroform, carbon tetrachloride and the like, at a suitable temperature, such as from about 0° C. to about 50° C.

Alternatively, a compound of Formula C9.2:

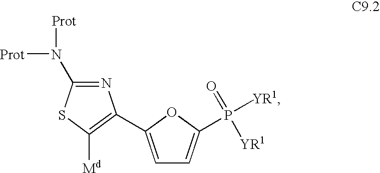

wherein Y and R¹ are defined as above, M^{d} is a metal selected from the group consisting of lithium, magnesium, zinc and copper, and 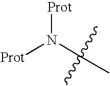
is a protected amino group,

is coupled to a compound of formula R¹¹—C(O)—X^{9b}, wherein R¹¹ is defined as above, and X^{9b} is halo, —O—C(O)—R¹, or alkylsulfonyloxy or arylsulfonyloxy.

When X^{9b} is halo, useful values of X^{9b} include F, Cl, Br and I, particularly Cl and Br.

When X^{9b} is alkylsulfonyloxy or arylsulfonyloxy, useful values of X^{9b} include methanesulfonyloxy, trifluoromethanesulfonyloxy and p-toluenesulfonyloxy.

More useful values of X^{9b} include halo.

The reaction can be carried out in a suitable solvent, such as THF, ether, DME, dioxane and toluene and the like, at a suitable temperature, such as from about 0° C. to about −78° C.

Examples of use of the method of the invention includes the following. It will be understood that these examples are exemplary and that the method of the invention is not limited solely to these examples.

For the purposes of clarity and brevity, chemical compounds are referred to by synthetic Example number in the biological examples below.

Besides the following Examples, assays that may be useful for identifying compounds which inhibit gluconeogenesis include the following animal models of diabetes:

i. Animals with pancreatic beta-cells destroyed by specific chemical cytotoxins such as Alloxan or Streptozotocin (e.g. the Streptozotocin-treated mouse, rat, dog, and monkey). Kodama, H., Fujita, M., Yamaguchi, I., Japanese Journal of Pharmacology 66:331-336 (1994) (mouse); Youn, J. H., Kim, J. K., Buchanan, T. A., Diabetes 43:564-571 (1994) (rat); Le Marchand, Y., Loten, E. G., Assimacopoulos-Jannet, F., et al., Diabetes 27:1182-88 (1978) (dog); and Pitkin, R. M., Reynolds, W. A., Diabetes 19:70-85 (1970) (monkey).

ii. Mutant mice such as the C57BL/Ks db/db, C57BL/Ks ob/ob, and C57BL/6J ob/ob strains from Jackson Laboratory, Bar Harbor, and others such as Yellow Obese, T-KK, and New Zealand Obese. Coleman, D. L., Hummel, K. P., Diabetologia 3:238-248 (1967) (C57BL/Ks db/db); Coleman, D. L., Diabetologia 14:141-148 (1978) (C57BL/6J ob/ob); Wolff, G. L., Pitot, H. C., Genetics 73:109-123 (1973) (Yellow Obese); Dulin, W. E., Wyse, B. M., Diabetologia 6:317-323 (1970) (T-KK); and Bielschowsky, M., Bielschowsky, F. Proceedings of the University of Otago Medical School 31:29-31 (1953) (New Zealand Obese).

iii. Mutant rats such as the Zucker fa/fa Rat rendered diabetic with Streptozotocin or Dexamethasone, the Zucker Diabetic Fatty Rat, and the Wistar Kyoto Fatty Rat. Stolz, K. J., Martin, R. J. Journal of Nutrition 112:997-1002 (1982) (Streptozotocin); Ogawa, A., Johnson, J. H., Ohnbeda, M., McAllister, C. T., Inman, L., Alam, T., Unger, R. H., The Journal of Clinical Investigation 90:497-504 (1992) (Dexamethasone); Clark, J. B., Palmer, C. J., Shaw, W. N., Proceedings of the Society for Experimental Biology and Medicine 173:68-75 (1983) (Zucker Diabetic Fatty Rat); and Idida, H., Shino, A., Matsuo, T., et al., Diabetes 30:1045-1050 (1981) (Wistar Kyoto Fatty Rat).

iv. Animals with spontaneous diabetes such as the Chinese Hamster, the Guinea Pig, the New Zealand White Rabbit, and non-human primates such as the Rhesus monkey and Squirrel monkey. Gerritsen, G. C., Connel, M. A., Blanks, M. C., Proceedings of the Nutrition Society 40:237 245 (1981) (Chinese Hamster); Lang, C. M., Munger, B. L., Diabetes 25:434-443 (1976) (Guinea Pig); Conaway, H. H., Brown, C. J., Sanders, L. L. et al., Journal of Heredity 71:179-186 (1980) (New Zealand White Rabbit); Hansen, B. C., Bodkin, M. L., Diabetologia 29:713-719 (1986) (Rhesus monkey); and Davidson, I. W., Lang, C. M., Blackwell, W. L., Diabetes 16:395-401 (1967) (Squirrel monkey).

V. Animals with nutritionally induced diabetes such as the Sand Rat, the Spiny Mouse, the Mongolian Gerbil, and the Cohen Sucrose-Induced Diabetic Rat. Schmidt-Nielsen, K., Hainess, H. B., Hackel, D. B., Science 143:689-690 (1964) (Sand Rat); Gonet, A. E., Stauffacher, W., Pictet, R., et al., Diabetologia 1:162-171 (1965) (Spiny Mouse); Boquist, L., Diabetologia 8:274-282 (1972) (Mongolian Gerbil); and Cohen, A. M., Teitebaum, A., Saliternik, R., Metabolism 21:235-240 (1972) (Cohen Sucrose-Induced Diabetic Rat).

vi. Any other animal with one of the following or a combination of the following characteristics resulting from a genetic predisposition, genetic engineering, selective breeding, or chemical or nutritional induction: impaired glucose tolerance, insulin resistance, hyperglycemia, obesity, accelerated gluconeogenesis, increased hepatic glucose output.

### BIOLOGICAL EXAMPLES

Examples of use of the method of the invention include the following. It will be understood that these examples are exemplary and that the method of the invention is not limited solely to these examples.

For the purposes of clarity and brevity, chemical compounds are referred to as synthetic example numbers in the biological examples below.

### Example A

### Inhibition of Human Liver FBPase

E. coli strain BL21 transformed with a human liver FBPase-encoding plasmid was obtained from Dr. M. R. El-Maghrabi at the State University of New York at Stony Brook. The enzyme was typically purified from 10 liters of recombinant E. coli culture as described (M. Gidh-Jain et al., The Journal of Biological Chemistry 269:27732-27738 (1994)). Enzymatic activity was measured spectrophotometrically in reactions that coupled the formation of product (fructose 6-phosphate) to the reduction of dimethylthiazoldiphenyltetrazolium bromide (MTT) via NADP⁺ and phenazine methosulfate (PMS), using phosphoglucose isomerase and glucose 6-phosphate dehydrogenase as the coupling enzymes. Reaction mixtures (200 μl) were made up in 96-well microtitre plates, and consisted of 50 mM Tris-HCl, pH 7.4, 100 mM KCl, 5 mM EGTA, 2 mM MgCl₂, 0.2 mM NADP, 1 mg/ml BSA, 1 mM MTT, 0.6 mM PMS, 1 unit/ml phosphoglucose isomerase, 2 units/ml glucose 6-phosphate dehydrogenase, and 0.150 mM substrate (fructose 1,6-bisphosphate). Inhibitor concentrations were varied from 0.01 μM to 10 μM. Reactions were started by the addition of 0.002 units of pure hlFBPase, and were monitored for 7 min. at 590 nm in a Molecular Devices Plate Reader (37° C.).

The table below provides the IC₅₀ values for several compounds prepared. The IC₅₀ for AMP, one of the physiological regulators of the enzyme, was 1 μM under these conditions. Prodrugs and their metabolic intermediates (monoamidates, N-acetylated phosphonic acids) were poorly active in this assay. Many of the compounds profiled showed significantly greater potency than AMP (up to >80-fold).

| Compound # | IC₅₀ (hlFBPase), μM |
|---|---|
| 1.1 | 0.031 |
| N-acetyl-1.1 | 2.31 |
| 1.2 | 0.025 |
| 1.3 | 0.018 |
| 1.4 | 0.066 |
| 1.5 | 0.056 |
| 1.6 | 0.040 |
| 1.7 | 0.041 |
| 1.8 | 0.037 |
| 1.9 | 0.086 |
| 1.10 | 0.048 |
| 1.11 | 0.036 |
| 1.12 | 0.073 |
| 1.13 | 0.048 |
| 1.14 | 0.118 |
| 1.16 | 0.061 |
| 1.17 | 0.094 |
| 1.18 | 0.085 |
| 1.19 | 0.053 |
| 1.20 | 10 |
| 1.21 | 0.054 |
| 1.22 | 0.033 |
| 1.23 | 0.009 |
| 1.24 | 0.099 |
| 1.25 | 0.095 |
| 1.26 | 0.119 |
| 1.27 | 0.111 |
| 1.28 | 0.057 |
| 1.29 | 0.06 |
| 1.30 | 0.014 |
| 1.31 | 0.073 |
| 1.32 | 0.105 |
| 1.33 | 0.011 |
| 1.34 | 0.016 |
| 2.1 | >100 |
| 3.1 | 0.012 |
| 3.6 | 0.025 |
| N-acetyl-3.6 | >10 |
| 4.6 | >100 |

### Example B

### Inhibition of Rat Liver FBPase

Rat liver FBPase was obtained by homogenizing freshly isolated rat liver in 100 mM Tris-HCl buffer, pH 7.4, containing 1 mM EGTA, and 10% glycerol. The homogenate was clarified by centrifugation, and the 45-75% ammonium sulfate fraction prepared. This fraction was redissolved in the homogenization buffer and desalted on a PD-10 gel filtration column (Biorad) eluted with same. This partially purified fraction was used for enzyme assays. Rat FBPase were assayed as described for human liver FBPase in Example A. Generally, as reflected below by the higher IC₅₀ values, the rat liver enzyme is less sensitive to inhibition by the compounds tested than the human liver enzyme.

| Compound # | IC₅₀ (rlFBPase), μM |
|---|---|
| 1.1 | 0.189 |
| 3.1 | 0.092 |
| 3.6 | 0.14 |

### Example C

### N-acetylation by Recombinant Human NAT1 and NAT2

Insect cell-expressed human NAT1 and NAT2 and control insect cytosol were obtained from BD Gentest (Bedford, Mass.). Compounds (100 μM) were incubated in 0.25 mL of NAT reaction cocktail consisting of 25 mM potassium phosphate pH 7.4 (at 25° C.), 1 mM EDTA, 1 mM DTT, 0.5 mM acetyl CoA, 5 mM acetyl-DL-carnitine, 20 u/mL acetyltransferase and either NAT1, NAT2 or control insect cytosol (0.1 mg/mL). Reactions were performed in an Eppendorf Thermomixer (37° C., 120 min.). At 0 and 120 min., 100 μl of each reaction was removed and added to a clean 1.7 mL tube containing 150 μl of 100% methanol. The tubes were vortexed and centrifuged at 14,000 rpm for 10 min. in an Eppendorf microcentrifuge (room temperature, 5 min., 14,000 rpm). The supernatants were analyzed by HPLC (Agilent 1100 series) using a Phenosphere C18 column (5 micron, 150×4.6 mm). The column was equilibrated with 20 mM potassium phosphate pH 4.5 or pH 6.2 (at 25° C.) and eluted with a linear gradient to 80% acetonitrile. The percent conversion of the compounds was calculated from the following equation: area of N-acyl product divided by (area of compound+area of N-acyl product), multiplied by 100.

Several of the compounds prepared had low or undetectable rates of N-acetylation (see table below). N-acetylation is a measure of metabolic stability. The intestine (site of drug absorption) and the liver (potential site of drug metabolism and clearance) are known to express N-acetylase activity. N-acetylation of the free phosphonic acids (the active moieties) generally results in a loss of potency. N-acetylation of compound 1.1 to N-acetyl-1.1, for instance, resulted in a 74.5-fold rightward shift in potency in the FBPase assay (Example A). Phosphonic acids (e.g. 1.2) that do not undergo N-acetylation have a longer half-life in the liver (the main site in which glucose is produced in the body via gluconeogenesis). N-acetylation of prodrugs results in the formation of a species that is converted to the N-acetylated, less active form of the phosphonic acid FBPase inhibitor in liver.

% Conversion

| Compound # | NAT1 | NAT2 |
|---|---|---|
| 1.1 | 0 | 0 |
| 1.2 | 0 | |
| 1.3 | 0 | |
| 2.1 | 0 | 0 |
| 2.2 | | 0 |
| 2.3 | | 0 |
| 3.1 | 22.4 | |
| 3.2 | 22.3 | |
| 3.3 | 4.6 | |
| 3.6 | 51 | 5.9 |
| 3.4 | 14.0 | |
| 3.5 | 14.2 | |
| 4.1 | 3.1 | 11.6 |
| 4.2 | | 24.9 |
| 4.3 | | 28 |
| 4.4 | | 100 |
| 4.5 | | 16.5 |
| 4.6 | 6.3 | 62.4 |

### Example D

### Prodrug Conversion to Active Moiety in Liver S9

Compounds were incubated at 100 μM in 1.0 mL of rat, dog, monkey or human liver S9 cocktail in an Eppendorf Thermomixer (37° C., 120 min.). At 0, 5, 15, 30, 60, and 120 min., aliquots (100 μL) of each reaction were removed and extracted in methanol as described in Example C. Conversion of 4.6 and 2.1 to 3.6 and 1.1, respectively, was assessed by reverse phase HPLC as described in Example C. Synthetic standards prepared in the appropriate methanol-extracted liver S9 fraction were used to generate calibration curves. Conversion rates were calculated from the initial, linear portion of time versus concentration curves.

As shown in the table below, the rate of conversion of 2.1 to the active moiety (1.1) was 1.6- to 4-fold more rapid than the conversion of 4.6 to 3.6 in the liver S9 fractions of the four species examined. A higher rate of prodrug conversion in the liver leads to higher exposure of liver to the active moiety. High liver exposure is expected to be associated with improved inhibition of gluconeogenesis and glucose lowering in type 2 diabetics.

| | Conversion to active moiety, nmoles/min/mg protein | | | |
|---|---|---|---|---|
| | Rat S9 | Dog S9 | Monkey S9 | Human S9 |
| 4.6 | 0.014 ± 0.007 | 0.008 ± 0.001 | 0.016 ± 0.004 | 0.006 ± 0.000 |
| 2.1 | 0.023 ± 007 | 0.032 ± 0.001 | 0.037 ± 0.008 | 0.010 ± 0.000 |

### Example E

### Liver Levels of Active Moiety Following Oral Prodrug Administration

Compound 4.6 and compound 2.1 were administered to Sprague-Dawley rats (250-300 g; n=3/group) via oral gavage in a polyethylene glycol-400 formulation at a dose of 30 mg/kg. At 3 h following drug administration, the animals were anesthetized and liver biopsies were taken. The liver samples were homogenized in 10% perchloric acid, neutralized, and analyzed for compound 3.6 or compound 1.1 concentration by reverse phase HPLC as described in Example C.

Compound 2.1 generated significantly higher levels of its respective active moiety in liver (13.5±2.4 nmoles/g) than did 4.6 (5.9±1.1 nmoles/g). This may be a consequence of improved distribution of compound 2.1 to liver (possibly due to improved bioavailability) and/or the higher rate of conversion of compound 2.1 to 1.1 in liver (Example D). Higher levels of active moiety in liver following treatment with compound 2.1 will result in more profound glucose lowering in type 2 diabetics.

### Example F

### Monoamidate Intermediate Levels in Plasma Following Oral Administration of Prodrugs to the Rat

Compounds 4.6 and 2.1 were administered at a dose of 30 mg/kg (suspension formulation in 0.1% carboxymethylcellulose) by oral gavage to 18-h fasted, Sprague-Dawley rats (250-300 g; n=3/group) instrumented with tail vein catheters. At various time points up to 24 h following drug administration, blood samples were taken from the tail vein and plasma was prepared by centrifugation (Eppendorf Microfuge, 14,000 rpm, 2 min, room temperature). Plasma samples were analyzed for the active moiety generated (3.6 and 1.1 were analyzed for 4.6 and 2.1, respectively) as well as for the formation of the monoamidate intermediate in prodrug conversion. Analysis was by HPLC as described in Example C for 4.6 and by LC-MS/MS as described in Example 1 for 2.1. The area under the curve (AUC) for the active moieties and monoamidate intermediates was determined by trapezoidal summation of the plasma concentration-time profile to the last measurable time point using WinNonLin version 1.1 software (Scientific Consulting, Inc., Cary, N.C.).

Following compound 4.6 administration, the AUC values for 3.6 and the monoamidate intermediate were 9.45±1.76 and 5.36±1.32 mg*kg/L, respectively. Following 2.1 administration, the AUC values for 1.1 and the corresponding monoamidate intermediate were 6.4 and 1.56±0.66 mg*kg/L, respectively. The ratios of active moiety to monoamidate intermediate for 4.6 and 2.1 are thus 1.8 and 4.1, respectively.

The reduced AUC of monoamidate intermediate in plasma suggests that 2.1 is converted more efficiently to active moiety in vivo than is 4.6. This is likely a reflection of the higher rate of 2.1 conversion in liver relative to 4.6 (Examples D and E). Although no known toxicities are associated with the intermediates in vivo, reduced exposure to intermediate may provide an advantage to 2.1 in terms of long term safety in type 2 diabetics. Drug intermediates can potentially build up in vivo when clearance pathways are saturated. This may lead to the accumulation of intermediates to high levels. High intermediate levels increase the likelihood of undesirable interactions with physiological processes.

### Example G

### Glucose Lowering Following Oral Administration to the Fasted, Normal Rat

Compounds were administered by oral gavage to 18-h fasted, Sprague-Dawley rats (250-300 g, n=3/4/group) at a dose of 10 mg/kg. Phosphonic acids (active moieties) were prepared in deionized water, and the solution adjusted to neutrality with sodium hydroxide. Prodrugs were dissolved in polyethylene glycol (mw 400). Blood samples were taken via tail vein nick immediately prior to dosing and at 1 h intervals thereafter. Blood glucose was analyzed by means of a HemoCue glucose analyzer (HemoCue Inc., Mission Viejo, Calif.). The table below indicates the maximum % glucose lowering achieved relative to control animals dosed with saline.

| Compound # | % Glucose Lowering | Time point, h |
|---|---|---|
| 2.1 | 68 | 3 |
| 2.2 | 25 | 3 |
| 2.3 | 27 | 1.5 |
| 2.4 | 22 | 3 |
| 2.5 | 15 | 1.5 |
| 2.6 | 12 | 3 |
| 2.7 | 10 | 3 |
| 2.8 | 14 | 1.5 |
| 2.9 | 24 | 1.5 |
| 2.10 | 12 | 5 |
| 2.11 | 13 | 5 |
| 2.12 | 10 | 1.5 |
| 2.14 | 20 | 1.5 |
| 2.15 | 8 | 1.5 |
| 2.16 | 6 | 1.5 |
| 2.17 | 10 | 1.5 |
| 2.18 | 13 | 1.5 |
| 2.19 | 16 | 1.5 |
| 2.20 | 6 | 1.5 |
| 2.21 | 9 | 1.5 |
| 2.22 | 9 | 1.5 |
| 2.23 | 12 | 1.5 |
| 2.24 | 19 | 1.5 |
| 2.25 | 10 | 3.0 |
| 2.26 | 16 | 1.5 |
| 2.27 | 6 | 1.5 |
| 2.28 | 10 | 1.5 |
| 2.29 | 12 | 3.0 |
| 5.1 | 8 | 1.5 |
| 5.3 | 51 | 3.0 |
| 5.4 | 16 | 1.5 |
| 5.5 | 20 | 1.5 |
| 5.6 | 4 | 3.0 |
| 5.7 | 25 | 1.5 |
| 5.8 | 10 | 3.0 |
| 5.9 | 10 | 1.5 |
| 5.10 | 4 | 3.0 |
| 5.12 | 38 | 3.0 |
| 6.2 | 3 | 5.0 |
| 6.4 | 14 | 5.0 |
| 6.5 | 9 | 3.0 |
| 6.6 | 4 | 3.0 |
| 6.8 | 1 | 5.0 |
| 7.1 | 70 | 3.0 |
| 7.2 | 3 | 1.5 |
| 8.2 | 10 | 1.5 |
| 8.3 | 24 | 1.5 |
| 8.4 | 6 | 5.0 |

### Example H

### Estimation of the Oral Bioavailability Based on Urinary Excretion

Phosphonic acids were dissolved in water, and the solution adjusted to neutrality with sodium hydroxide. Prodrugs were dissolved in 10% ethanol/90% polyethlene glycol (mw 400). Compound was administered by oral gavage to 18-h fasted Sprague-Dawley rats (220-250 g) at doses ranging from 10-50 mg/kg. The rats were subsequently placed in metabolic cages and urine was collected for 24 h. The quantity of phosphonic acid (active moiety) excreted into urine was determined by HPLC analysis as described in Example C. In a separate study, urinary recovery was determined following intravenous (tail vein) administration of compound (in the case of the prodrugs, the appropriate parent phosphonic acid was administered i.v.). The percentage oral bioavailability was estimated by comparison of the recovery of compound in urine 24 h following oral administration, to that recovered in urine 24 h after intravenous administration.

The oral bioavailabilities of select phosphonic acids, and prodrugs of phosphonic acids are shown in the table below. Compound 2.1 had the highest oral bioavailability. High oral bioavailability is expected to result in improved potency and reduced interindividual variability in type 2 diabetics.

| Compound # | % Oral bioavailability |
|---|---|
| 2.1 | 30 |
| 2.2 | 11 |
| 2.5 | 4 |
| 4.1 | 24 |
| 4.6 | 21 |

### Example I

### Estimation of Oral Bioavailability Based on Plasma Drug Levels

Sprague-Dawley rats (250-300 g; n=3/group) were instrumented with tail vein and artery catheters at 8 am and allowed to recover for at least 2 h. One group was administered compound 2.1 at a dose of 30 mg/kg in polyethylene glycol-400 by gavage. In a second group, intravenous PK was assessed following administration of compound 2.1 dissolved in 25% hydroxypropyl β-cyclodextrin at a dose of 10 mg/kg. Blood samples were obtained from the tail artery catheter at regular time intervals and collected into heparinized microfuge tubes. Plasma was prepared by centrifugation (1 min., 14,000 rpm, RT, Eppendorf microfuge).

Plasma samples (50 μL) were diluted with 50% acetonitrile in water (10 μL) and the plasma proteins were precipitated by the addition of 100% acetonitrile (75 μL). After 20 min. of centrifugation (Eppendorf microfuge, 14,000 rpm, RT) the resulting supernatant was analyzed by LC-MS/MS (Applied Biosystems, API 4000 equipped with an Agilent 1100 binary pump and a LEAP injector). The sample (10 μL) was injected onto an Xterra MS C18 column (3.5 um, 2.1×50 mm, Waters Corp.) with a SecurityGuard C18 guard column (5 μm, 4.0×3.0 mm, Phenomenex) and eluted with a gradient from mobile phase A (10 mM ammonium acetate in 5% acetonitrile in de-ionized water) to B (50% acetonitrile in de-ionized water) at a flow rate of 0.3 mL/min (0 min, 10% B; 0-1 min, 0-100% B; 1-6 min, 100% B; 6-6.1 min, 100-10% B; 6.1-9 min, 10% B). The injector temperature was set at 4° C. The elution time for 1.1 was approximately 2.9 min. 1.1 was detected in MS/MS mode (331.1/247) and quantified by comparison of peak areas to standard curves obtained by spiking known concentrations of 1.1 into blank rat plasma. Calibration curves ranging from 10 to 3000 ng/mL of 1.1 were generated. The LOQ for 1.1 was 10 ng/mL.

The plasma concentration-time data were analyzed by non-compartmental methods using WinNonLin version 1.1 (Scientific Consulting, Inc., Cary, N.C.). The area under the curve (AUC) was determined by trapezoidal summation of the plasma concentration-time profile to the last measurable time point. For IV bolus analysis, back extrapolation of the plasma concentration-time plot was performed to estimate the zero time intercept by fitting a natural log-linear line to the first two data points.

The AUC values of 1.1 following oral and IV administration were 10.85±0.77 and 9.27±0.78 mg·h/L, respectively. Based on the comparison of the dose-normalized AUC values of the plasma concentration-time profile of 1.1 following oral dosing of prodrug with the AUC values of 1.1 following IV administration of prodrug, the oral bioavailability of 2.1 was estimated to be 39%.

### Example J

### Blood Glucose Lowering in the Zucker Diabetic Fatty (ZDF) Rat

Nine week old ZDF rats were fasted at 7 am and screened for hyperglycemia (BG>300 mg/dl) at 12:30 pm. Rats were divided into 3 glucose-matched groups and dosed with compound 4.6 or compound 2.1 at 10 mg/kg in PEG-400 or an equal volume of vehicle (n=8/group) at 1 pm. Food was withheld for 6 to 9 h. Blood samples were collected intermittently from the tail vein and diluted 1:2 (v:v) in 20% glycerol-saline with 20 U/ml heparin. Blood glucose was determined by means of a Hemocue glucose analyzer (Hemocue Inc., Mission Viejo, Calif.) used according to the instructions of the manufacturer. Results were expressed as means ±standard errors of the mean (sem) for all values. Differences between treatment and vehicle-treated animals were evaluated using ANOVA with Dunnett's post-hoc analysis or Tukey-Kramer's post hoc analysis when all differences are compared. Differences are considered significant when p≦0.05.

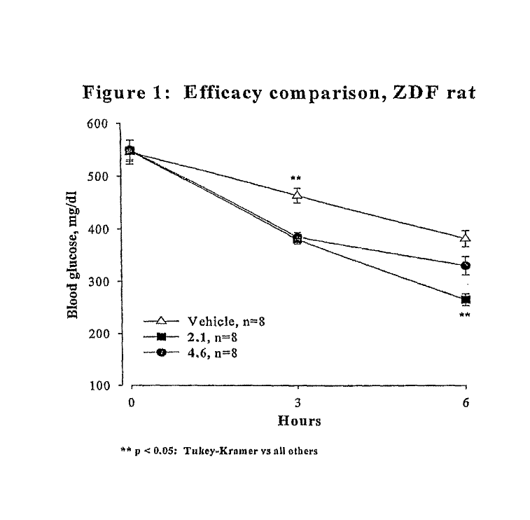

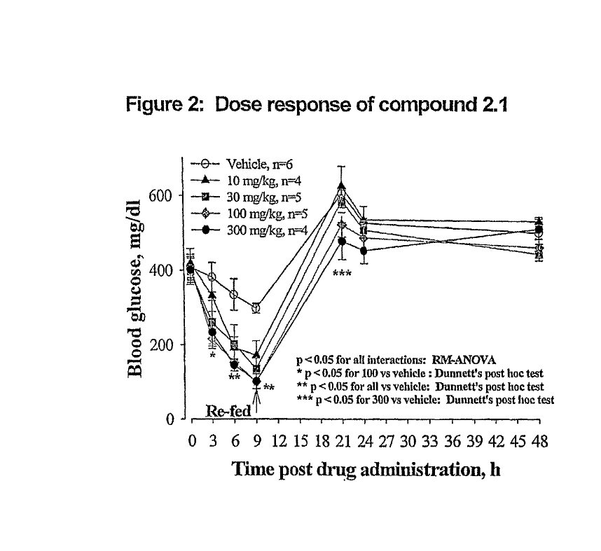

Compound 2.1 showed significantly more sustained glucose lowering than 4.6: 30% (p<0.05) vs. 14% (ns) at 6 h compared to vehicle-treated rats, respectively (FIG. 1). In a follow-up study in the ZDF rat using a similar protocol, compound 2.1 (at a range of doses: 10-300 mg/kg) was found to have a duration of action of >9 h in this model (FIG. 2).

The ZDF rat is a well-characterized model of type 2 diabetes. The nature and progression of the disease closely parallels that in humans. The extended duration of action of compound 2.1 relative to 4.6 in this animal model suggests that compound 2.1 may more effectively treat type 2 diabetes in humans.

### Example K

### Variability of N-acetylation in Human Liver S9 Fractions

Compounds 4.6, 3.6, 2.1, or 1.1 (100 μM) are incubated in 0.25 mL of reaction cocktail consisting of 25 mM potassium phosphate pH 7.4 (at 25° C.), 1 mM EDTA, 1 mM DTT, 0.5 mM acetyl CoA, 5 mM acetyl-DL-carnitine, 20 μ/mL acetyltransferase and human liver S9 (final concentration 10 mg/mL protein) from various donors (e.g. catalog nos. 452801, 452835, 452847, 452864; Gentest, Woburn, Mass.). The reactions are incubated, processed, and analyzed as described in Example C.

Compounds 4.6 and 3.6 generate high levels of the corresponding N-acetylated metabolite in human liver S9 from some donors with high N-acetylase activity and low levels in that obtained from donors with low N-acetylase activity. Compounds 2.1 and 1.1 are stable under the reaction conditions; no conversion to N-acetylated metabolites is observed in S9 from donors with either high or low N-acetylase activity. The high inter-individual variability of N-acetylation of 4.6 and 3.6 results in a variable and unpredictable pharmacological response in human type 2 diabetics. In patients with high N-acetylase activity, poor glycemic control is obtained following treatment with 4.6, whereas in a subset of patients with low N-acetylase activity, adequate glycemic control is obtained. There is significantly reduced inter-individual variability in patients treated with 2.1 due to the insusceptibility of 2.1 and its active moiety (1.1) to N-acetylation. The metabolic stability of 2.1 and 1.1 translates to a high response rate, improved glycemic control, and predictable pharmacokinetics/pharmacodynamic in type 2 diabetic patients treated with 2.1.

### Example L

### Oral Bioavailability Determinations in the Monkey

Cynomolgus monkeys (3-3.6 kg) were dosed orally with vehicle or 2.1 in 100% PEG-400 (at 3, 10, 30 mg/kg) formulations, or intravenously with 1.1 in 25% hydroxypropyl β-cyclodextrin (HP-βCD) formulation (at 3 and 10 mg/kg). The dosing volumes were 10 mL/kg for oral administrations and 4 mL/kg for intravenous administrations. Animals were fasted overnight prior to oral dosing and were in the fed state for intravenous administrations. Blood samples were taken predose, and at 1, 2, 4, 6, 8, 12, and 24 h following oral administration, and at predose, 20 min., 1, 2, 4, 6, 8, and 12 h following intravenous administration. The samples were transferred to EDTA-containing tubes and stored on an ice block until centrifuged (3000 g, 5-10 min.). Following centrifugation, the plasma supernatant was collected, transferred to a plastic vial, capped, and stored at −80° C.

On the day of analysis, plasma samples were thawed at room temperature. Thawed samples (50 μL) were diluted with 50% acetonitrile in water (10 μL) and the plasma proteins precipitated by addition of 100% acetonitrile (75 μL). After 20 min. of centrifugation (Eppendorf microfuge, 14,000 rpm, RT) the resulting supernatant was collected and analyzed using an LC-MS/MS (Applied Biosystems, API 4000) equipped with an Agilent 1100 binary pump and a LEAP injector. Ten μL of sample was injected onto an Xterra MS C18 column (3.5 um, 2.1×50 mm, Waters Corp.) fitted with a SecurityGuard C18 guard column (5 μm, 4.0×3.0 mm, Phenomenex) and eluted with a gradient from mobile phase A (10 mM ammonium acetate in 5% acetonitrile in de-ionized water) to B (50% acetonitrile in de-ionized water) at a flow rate of 0.3 mL/min (0 min, 10% B; 0-1 min, 0-100% B; 1-6 min, 100% B; 6-6.1 min, 100-10% B; 6.1-9 min, 10% B). The injector temperature was set at 4° C. Elution times for 2.1 and 1.1 were approximately 6.2 and 2.9 min, respectively. Compounds 2.1 and 1.1 were detected using the MS/MS mode (557.6/231.2 for 2.1 and 331.3/247.2 for 1.1) and quantified by comparison of peak areas to standard curves obtained by spiking known concentrations of the analytes to blank monkey plasma. Calibration curves ranging from 10 to 3000 ng/mL of 2.1 and 1.1 were generated. The limit of quantitation (LOQ) for both 2.1 and 1.1 was 10 ng/mL.

The temporal plasma concentration data was analyzed by non-compartmental methods. Oral bioavailability (OBAV) was estimated by comparison of the dose-normalized AUC values of the plasma profile of 1.1 following oral and iv administration of 2.1 in each individual monkey. The OBAV of 2.1 was excellent; it ranged from 54.3 to 65.3% for the three doses. High oral bioavailability in the monkey is predictive of good pharmacokinetic properties (e.g. good absorption and low inter-individual variability) in humans.

Having now fully described this invention, it will be understood by those of ordinary skill in the art that the same can be performed within a wide and equivalent range of conditions, formulations and other parameters without affecting the scope of the invention or any embodiment thereof.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All documents (e.g., scientific publications, patents and patent publications) recited herein are hereby incorporated by reference in their entirety to the same extent as if each individual document was specifically and individually indicated to be incorporated by reference in its entirety. Where the document cited only provides the first page of the document, the entire document is intended, including the remaining pages of the document.

What is claimed is:

1. A compound of Formula (I) or a salt thereof: 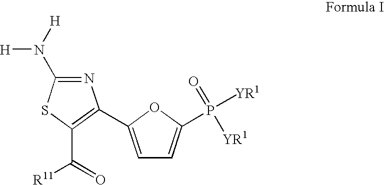
wherein:
R¹¹ is selected from the group consisting of C₁-C₂₀ alkyl, C₁-C₂₀ cycloalkyl, monocyclic aryl, bicyclic aryl, monocyclic heteroaryl and bicyclic heteroaryl, optionally substituted with halogen, OH, C₁-C₄ alkoxy, cyano, alkyl, aryl, NR³₂, NR⁴₂, morpholino, pyrrolidinyl, NMe₂ and perhaloalkyl;
Y is independently selected from the group consisting of —O—, and —NR⁶—;
when Y is —O—, then R¹ attached to —O— is independently selected from the group consisting of —H, optionally substituted aryl, optionally substituted-alkylaryl, —C(R²)₂OC(O)NR²₂, —NR²—C(O)—R³, —C(R²)₂—OC(O)R³, —C(R²)₂—O—C(O)OR³, —C(R²)₂OC(O)SR³, -alkyl-S—C(O)OR³ and -alkyl-S—C(O)R³;
when Y is —NR⁶—, then R¹ attached to —NR⁶— is independently selected from the group consisting of —H, —[C(R²)₂]_{q}—COOR³, —C(R⁴)₂COOR³,
—[C(R²)₂]_{q}—C(O)SR, and -cycloalkylene-COOR³;
or when one Y—R¹ is —NR¹⁵(R¹⁶) then the other Y—R¹ is —N(R¹⁸)—(CR¹²R¹³)ₙ—C(O)—R¹⁴;
or both Y—R¹ are —N(R¹⁸)—(CR¹²R¹³)ₙ—C(O)—R¹⁴;
or when either Y is independently selected from —O— and —NR⁶—, then together R¹ and R¹ are 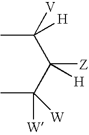
wherein
V, W, and W′ are independently selected from the group consisting of hydrogen, optionally substituted alkyl, optionally substituted aralkyl, heterocycloalkyl, aryl, substituted aryl, heteroaryl, substituted heteroaryl, optionally substituted 1-alkenyl, and optionally substituted 1-alkynyl; or
together V and Z are connected via an additional 3-5 atoms to form a cyclic group containing 5-7 atoms, optionally 1 heteroatom, substituted with hydroxy, acyloxy, alkoxycarbonyloxy, or aryloxycarbonyloxy attached to a carbon atom that is three atoms from both Y groups attached to the phosphorus; or
together V and Z are connected via an additional 3-5 atoms to form a cyclic group, optionally containing 1 heteroatom, that is fused to an aryl group at the beta and gamma position to the Y attached to the phosphorus; or
together V and W are connected via an additional 3 carbon atoms to form an optionally substituted cyclic group containing 6 carbon atoms and substituted with one substituent selected from the group consisting of hydroxy, acyloxy, alkoxycarbonyloxy, alkylthiocarbonyloxy, and aryloxycarbonyloxy, attached to one of said carbon atoms that is three atoms from a Y attached to the phosphorus; or
together Z and W are connected via an additional 3-5 atoms to form a cyclic group, optionally containing one heteroatom, and V must be aryl, substituted aryl, heteroaryl, or substituted heteroaryl; or
together W and W′ are connected via an additional 2-5 atoms to form a cyclic group, optionally containing 0-2 heteroatoms, and V must be aryl, substituted aryl, heteroaryl, or substituted heteroaryl;
Z is selected from the group consisting of —CHR²OH, —CHR²OC(O)R³, —CHR²OC(S)R³, —CHR²OC(S)OR³, —CHR²OC(O)SR³, —CHR²OCO₂R³, —OR², —SR², —CHR²N₃, —CH₂aryl, —CH(aryl)OH, —CH(CH═CR²₂)OH, —CH(C≡CR²)OH, —R², —NR²₂, —OCOR³, —OCO₂R³, —SCOR³, —SCO₂R³, —NHCOR², —NHCO₂R³, —CH₂NHaryl, —(CH₂)ₚ—OR², and —(CH₂)ₚ—SR²;
n is an integer from 1 to 3;
p is an integer 2 or 3;
q is an integer 1 or 2;
with the provisos that:
a) V, Z, W, W′ are not all —H; and
b) when Z is —R², then at least one of V, W, and W′ is not —H, alkyl aralkyl, or heterocycloalkyl;
R² is selected from the group consisting of R³ and —H;
R³ is selected from the group consisting of alkyl, aryl, heterocycloalkyl, and aralkyl;
each R⁴ is independently selected from the group consisting of —H and alkyl, or
together R⁴ and R⁴ form a cyclic alkyl group;
R⁶ is selected from the group consisting of —H, lower alkyl, acyloxyalkyl, alkoxycarbonyloxyalkyl, and lower acyl;
each R¹² and R¹³ is independently selected from the group consisting of H, lower alkyl, lower aryl, and lower aralkyl, all optionally substituted, or R¹² and R¹³ together are connected via 2-6 atoms, optionally including 1-2 heteroatoms selected from the group consisting of O, N and S, to form a cyclic group;
each R¹⁴ is independently selected from the group consisting of —OR¹⁷, —N(R¹⁷)₂, —NHR¹⁷, —NR²⁰R¹⁹ and —SR¹⁷;
R¹⁵ is selected from the group consisting of —H, lower alkyl, lower aryl and lower aralkyl, or together with R¹⁶ is connected via 2-6 atoms, optionally including 1 heteroatom selected from the group consisting of O, N, and S;
R¹⁶ is selected from the group consisting of —(CR¹²R¹³)ₙ—C(O)—R¹⁴, —H, lower alkyl, lower aryl and lower aralkyl, or together with R¹⁵ is connected via 2-6 atoms, optionally including 1 heteroatom selected from the group consisting of O, N, and S;
each R¹⁷ is independently selected from the group consisting of lower alkyl, lower aryl, and lower aralkyl, all optionally substituted, or together R¹⁷ and R¹⁷ on N is connected via 2-6 atoms, optionally including 1 heteroatom selected from the group consisting of O, N, and S;
R¹⁸ is independently selected from the group consisting of H, lower alkyl, aryl, and aralkyl, or together with R¹² is connected via 1-4 carbon atoms to form a cyclic group;
each R¹⁹ is independently selected from the group consisting of —H, lower alkyl, lower aryl, lower heterocycloalkyl, lower aralkyl, and COR³;
or a pharmaceutically acceptable prodrug or salt thereof.

2. The compound of claim 1 wherein Y is independently selected from the group consisting of —O—, and —NR⁶—;
or when one Y—R¹ is —NR¹⁵(R¹⁶) then the other Y—R¹ is —N(R¹⁸)—(CR¹²R¹³)ₙ—C(O)—R¹⁴;
or both Y—R¹'s are —N(R¹⁸)—(CR¹²R¹³)ₙ—C(O)—R¹⁴;
or when Y is —O—, then R¹ attached to —O— is independently selected from the group consisting of —H, —C(R²)₂—OC(O)R³, and —C(R²)₂—O—C(O)OR³,
or when Y is —NR⁶—, then R¹ attached to —NR⁶— is independently selected from the group consisting of —H, —[C(R²)₂]_{q}—COOR³, —C(R⁴)₂COOR³,
—[C(R²)₂]_{q}—C(O)SR, and -cycloalkylene-COOR³;
or when both Y's are —O—, then together R¹ and R¹ are 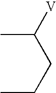
wherein
V is selected from the group consisting of optionally substituted monocyclic aryl and optionally substituted monocyclic heteroaryl.

3. The compound of claim 2, wherein both Y groups are —O—.

4. The compound of claim 2, wherein one Y is —NR⁶—, and one Y is —O—.

5. The compound of claim 1, wherein when Y is O, R¹ is independently selected from the group consisting of optionally substituted aryl, optionally substituted benzyl, —C(R²)₂OC(O)R³, —C(R²)₂OC(O)OR³, and —H; and
when Y is —NR⁶—, then the R¹ attached to said —NR⁶— group is selected from the group consisting of —C(R⁴)₂—COOR³, and —C(R²)₂COOR³; and the other Y group is —O— and then R¹ attached to said —O— is selected from the group consisting of optionally substituted aryl, —C(R²)₂OC(O)R³, and —C(R²)₂OC(O)OR³.

6. The compound of claim 1, wherein Y is O and R¹ is H.

7. The compound of claim 1, wherein one Y—R¹ is —NR¹⁵(R¹⁶) and the other Y—R¹ is —N(R¹⁸)—(CR¹²R¹³)ₙ—C(O)—R¹⁴.

8. The compound of claim 1, wherein both Y—R¹'s are —N(R¹⁸)—(CR¹²R¹³)ₙ—C(O)—R¹⁴.

9. The compound of claim 8, wherein n is 1, R¹⁸ is H, and R¹⁴ is —OR³.

10. The compound of claim 8, wherein R¹² is H; R¹³ is methyl; and the carbon bearing R¹² and R¹³ is in the (S)-configuration.

11. The compound of claim 8, wherein R¹² is methyl and R¹³ is methyl.

12. The compound of claim 1, wherein R¹¹ is C₃-C₁₀ alkyl or cycloalkyl.

13. The compound of claim 2, wherein R¹¹ is C₃-C₁₀ alkyl or cycloalkyl.

14. The compound of claim 13, wherein R¹¹ is selected from the group consisting of methyl, ethyl, isopropyl, cyclobutyl, 3-pentyl and tert-butyl.

15. The compound of claim 13, wherein R¹¹ is selected from the group consisting of tert-butyl, 2-methyl-2-butyl, 3-methyl-3-pentyl, and 3-ethyl-3-pentyl.

16. The compound of claim 14, wherein when Y is —O—, then R¹ attached to —O— is independently selected from the group consisting of —H, —CH₂OC(O)-tBu, —CH₂OC(O)Et, and —CH₂OC(O)-iPr;
when Y is —NR⁶—, then R¹ is attached to —NR⁶— independently selected from the group consisting of —C(R²)₂COOR³ and —C(R⁴)₂COOR³; and
R⁶ is —H.

17. The compound of claim 14, wherein when Y is —O—, then R¹ attached to —O— is —H;
when Y is —NR⁶—, then R¹ attached to —NR⁶— is —C(R²)₂COOR³; and
R⁶ is —H.

18. The compound of claim 14, wherein when Y is —O—, then R¹ attached to —O— is —H;
when Y is —NR⁶—, then R¹ attached to —NR⁶— is —C(R²)₂COOR³;
R² is H or methyl;
R³ is ethyl or isopropyl; and
R⁶ is —H.

19. The compound of claim 14, wherein each YR¹ is —OH.

20. The compound of claim 14, wherein each YR¹ is —NHC(Me)₂COOEt.

21. The compound of claim 14, wherein R¹¹ is tert-butyl.

22. The compound of claim 14, wherein R¹¹ is isopropyl, 3-pentyl or cyclobutyl.

23. The compound of claim 15, wherein R¹¹ is 2-methyl-2-butyl.

24. The compound of claim 21, wherein when Y is —O—, then R¹ attached to —O— is independently selected from the group consisting of —H, optionally substituted phenyl, —CH₂OC(O)-tBu, —CH₂OC(O)OEt, and —CH₂OC(O)O-iPr;
when Y is —NR⁶—, then R¹ is attached to —NR⁶— independently selected from the group consisting of —C(R²)₂COOR³ and —C(R⁴)₂COOR³, or
when one Y—R¹ is —NR¹⁵(R¹⁶) then the other Y—R¹ is —N(R¹⁸)—(CR¹²R¹³)ₙ—C(O)—R¹⁴;
when Y is —O— or —NR⁶—, and at least one Y is —O—, then together R¹ and R¹ are 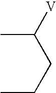
wherein
V is selected from the group consisting of optionally substituted aryl and optionally substituted heteroaryl;
R⁶ is selected from the group consisting of —H and lower alkyl.

25. The compound of claim 192, wherein when Y is —O—, then R¹ attached to —O— is independently selected from the group consisting of —H, —CH₂OC(O)-tBu, —CH₂OC(O)Et, and —CH₂OC(O)-iPr;
when Y is —NR⁶—, then R¹ is attached to —NR⁶— independently selected from the group consisting of —C(R²)₂COOR³ and —C(R⁴)₂COOR³; and
R⁶ is —H.

26. The compound of claim 21, wherein when Y is —O—, then R¹ attached to —O— is —H;
when Y is —NR⁶—, then R¹ attached to —NR⁶— is —C(R²)₂COOR³; and
R⁶ is —H.

27. The compound of claim 21, wherein when Y is —O—, then R¹ attached to —O— is —H;
when Y is —NR⁶—, then R¹ attached to —NR⁶— is —C(R²)₂COOR³;
R² is H or methyl;
R³ is ethyl or isopropyl; and
R⁶ is —H.

28. The compound of claim 21, wherein each YR¹ is —OH.

29. The compound of claim 22, wherein each YR¹ is —OH.

30. The compound of claim 22, wherein each YR¹ is —OH.

31. The compound of claim 21, wherein each YR¹ is —NHC(Me)₂COOEt.

32. The compound of claim 21, wherein each YR¹ is —NHCH(Me)COOEt.

33. The compound of claim 22, wherein each YR¹ is —NHC(Me)₂COOEt.

34. The compound of claim 22, wherein each YR¹ is —NHCH(Me)COOEt.

35. The compound of claim 24, wherein each YR¹ is —NHC(Me)₂COOEt.

36. The compound of claim 24, wherein each YR¹ is —NHCH(Me)COOEt.

37. The compound of claim 1, wherein said compound is: 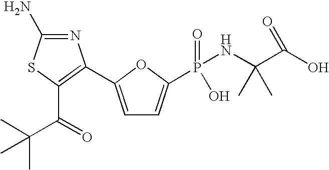
or a salt thereof.

38. The compound of claim 1, wherein said compound is 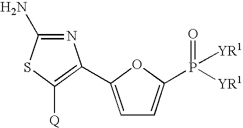
and Q and YR¹ are

| Compound # | Q | YR¹ | |
|---|---|---|---|
| 1.1 | 2,2-dimethylpropionyl | —OH | ; |
| 1.2 | 2,2-dimethylbutyryl | —OH | ; |
| 1.3 | 2-ethyl-2-methylbutyryl | —OH | ; |
| 1.4 | Acetyl | —OH | ; |
| 1.5 | Benzoyl | —OH | ; |
| 1.6 | cyclohexylcarbonyl | —OH | ; |
| 1.7 | 2-thienylcarbonyl | —OH | ; |
| 1.8 | 3-fluorobenzoyl | —OH | ; |
| 1.9 | 4-chlorobenzoyl | —OH | ; |
| 1.10 | 4-methylbenzoyl | —OH | ; |
| 1.11 | 3-methylbenzoyl | —OH | ; |
| 1.12 | 3-chlorobenzoyl | —OH | ; |
| 1.13 | 2-methylbenzoyl | —OH | ; |
| 1.14 | 2-methoxybenzoyl | —OH | ; |
| 1.15 | 2-chlorobenzoyl | —OH | ; |
| 1.16 | 4-methoxybenzoyl | —OH | ; |
| 1.17 | 3,4-dimethoxybenzoyl | —OH | ; |
| 1.18 | 3-methoxybenzoyl | —OH | ; |
| 1.19 | 3,4- | —OH | ; |
| | methylenedioxybenzoyl | | |
| 1.20 | 3-pyridylcarbonyl | —OH | ; |
| 1.21 | 3-chloro-4-(1-pyrrol- | —OH | ; |
| | idinyl)benzoyl | | |
| 1.22 | 4-fluorobenzoyl | —OH | ; |
| 1.23 | 2-ethylbutyryl | —OH | ; |
| 1.24 | 4-trifluoromethylbenzoyl | —OH | ; |
| 1.25 | 3-chloro-4-(1-morpho- | —OH | ; |
| | linyl)benzoyl | | |
| 1.26 | 3-trifluoromethylbenzoyl | —OH | ; |
| 1.27 | 2-trifluoromethylbenzoyl | —OH | ; |
| 1.28 | 4-phenylbenzoyl | —OH | ; |
| 1.29 | 2-naphthylcarbonyl | —OH | ; |
| 1.30 | cyclopentylcarbonyl | —OH | ; |
| 1.31 | 4-piperidinylbenzoyl | —OH | ; |
| 1.32 | 4-(N,N-dimethylamino)- | —OH | ; |
| | benzoyl | | |
| 1.33 | 2-methylbutyryl | —OH | ; |
| 1.34 | cyclobutylcarbonyl | —OH | ; |
| 2.1 | 2,2-dimethylpropionyl | —NHC(Me)₂CO₂Et | ; |
| 2.2 | 2,2-dimethylpropionyl | —NHCH(Me)CO₂Et (S) | ; |
| 2.3 | 2,2-dimethylpropionyl | —NHC(Me)₂CO₂i-Pr | ; |
| 2.4 | 2,2-dimethylpropionyl | —NHCH₂CO₂Et | ; |
| 2.5 | 2-ethyl-2-methylbutyryl | —NHC(Me)₂CO₂Et | ; |
| 2.6 | 2-ethyl-2-methylbutyryl | —NHC(Me)₂CO₂i-Pr | ; |
| 2.7 | 2-ethyl-2-methylbutyryl | —NHCH(Me)CO₂Et (S) | ; |
| 2.8 | 2,2-dimethylbutyryl | —NHC(Me)₂CO₂i-Pr | ; |
| 2.9 | 2,2-dimethylbutyryl | —NHC(Me)₂CO₂Et | ; |
| 2.10 | 2,2-dimethylbutyryl | —NHCH(Me)CO₂Et (S) | ; |
| 2.11 | 2-ethyl-2-methylbutyryl | —NHCH(Me)CO₂i-Pr (S) | ; |
| 2.12 | 2,2-dimethylbutyryl | —NHCH₂CO₂t-Bu | ; |
| 2.13 | 2,2-dimethylpropionyl | —NHCH(Me)CO₂i-Pr (S) | ; |
| 2.14 | 2-methylbenzoyl | —NHCH(Me)CO₂Et (S) | ; |
| 2.15 | 2-methylbenzoyl | —NHC(Me)₂CO₂Et | ; |
| 2.16 | 4-methylbenzoyl | —NHCH(Me)CO₂Et (S) | ; |
| 2.17 | 4-methylbenzoyl | —NHC(Me)₂CO₂Et | ; |
| 2.18 | 3-fluorobenzoyl | —NHCH(Me)CO₂i-Pr (S) | ; |
| 2.19 | 3-fluorobenzoyl | —NHCH(Me)CO₂Et (S) | ; |
| 2.20 | 4-methylbenzoyl | —NHC(Me)₂CO₂i-Pr | ; |
| 2.21 | 2-methylbenzoyl | —NHC(Me)₂CO₂i-Pr | ; |
| 2.22 | 2-methylbenzoyl | —NHCH(Me)CO₂i-Pr (S) | ; |
| 2.23 | 2-ethylbutyryl | —NHC(Me)₂CO₂Et | ; |
| 2.24 | 2-ethylbutyryl | —NHCH(Me)CO₂Et (S) | ; |
| 2.25 | 2-ethylbutyryl | —NHC(Me)₂CO₂i-Pr | ; |
| 2.26 | 2-ethylbutyryl | —NHCH(Me)CO₂i-Pr (S) | ; |
| 2.27 | 3-fluorobenzoyl | —NHC(Me)₂CO₂Et | ; |
| 2.28 | 3-fluorobenzoyl | —NHC(Me)₂CO₂i-Pr | ; |
| 2.29 | cyclobutylcarbonyl | —NHC(Me)₂CO₂Et | ; |
| 2.30 | cyclobutylcarbonyl | —NHCH(Me)CO₂Et (S) | ; |
| 2.31 | cyclobutylcarbonyl | —NHCH(Me)CO₂i-Pr (S) | ; |
| 2.32 | cyclobutylcarbonyl | —NHC(Me)₂CO₂i-Pr | ; |
| 3.1 | 2,2-dimethylpropyl | —OH | ; |
| 3.2 | cyclopentylmethyl | —OH | ; |
| 3.3 | 2,2-dimethylbutyl | —OH | ; |
| 3.4 | 2-propyl | —OH | ; |
| 3.5 | 2-methylbutyl | —OH | ; |
| 3.6 | 2-methylpropyl | —OH | ; |
| 4.1 | 2,2-dimethylpropyl | —NHCH(Me)CO₂Et (S) | ; |
| 4.2 | Phenyl | —NHCH(Me)CO₂Et (S) | ; |
| 4.3 | Cyclohexyl | —NHCH(Me)CO₂i-Pr (S) | ; |
| 4.4 | 2,2-dimethylpropyl | —NHCH₂CO₂Et | ; |
| 4.5 | 2,2,3-trimethylbutyl | —NHCH(Me)CO₂Et (S) | ; or |
| 4.6 | 2-methylpropyl | —NHCH(Me)CO₂Et (S) | ; |

and salts thereof.

39. The salt form of a compound of claim 1, wherein said salt form is selected from the group consisting of methanesulfonate, ethanesulfonate, sulfate, hydrochloride, hydrobromide, acetate, citrate and tartrate.

40. The salt form of a compound of claim 37, wherein said salt form is selected from the group consisting of methanesulfonate, ethanesulfonate, sulfate, hydrochloride, hydrobromide, acetate, citrate and tartrate.

41. The salt form of a compound of claim 38, wherein said salt form is selected from the group consisting of methanesulfonate, ethanesulfonate, sulfate, hydrochloride, hydrobromide, acetate, citrate and tartrate.

42. A pharmaceutical composition comprising a pharmaceutically effective amount of the compound of claim 1, or a pharmaceutically acceptable prodrug or salt thereof; and a pharmaceutically acceptable carrier.

43. A method of treating a disease or condition responsive to inhibition of gluconeogenesis or responsive to lowered blood glucose levels in an animal comprising administering to said animal a therapeutically effective amount of the compound of claim 1 or a pharmaceutically acceptable prodrug or salt thereof.

44. A method of treating diabetes in a patient comprising administering to said patient a therapeutically effective amount of the compound of claim 1 or a pharmaceutically acceptable prodrug or salt thereof.

45. A method of preventing diabetes in an animal comprising administering to an animal at risk of developing diabetes a therapeutically effective amount of the compound of claim 1 or a pharmaceutically acceptable prodrug or salt thereof.

46. A method of treating impaired glucose tolerance in a patient comprising administering to said patient a therapeutically effective amount of the compound of claim 1 or a pharmaceutically acceptable prodrug or salt thereof.

47. A method of treating insulin resistance in a patient comprising administering to said patient a therapeutically effective amount of the compound of claim 1 or a pharmaceutically acceptable prodrug or salt thereof.

48. A method of making a compound of claim 1, comprising:
deprotecting a compound of Formula C1.8: 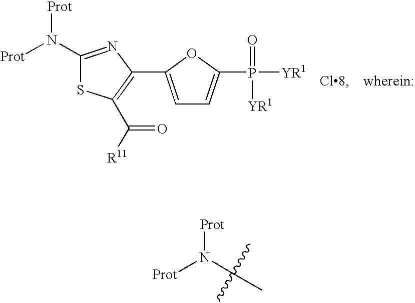
is a protected amino group; and
R¹, Y and R¹¹ are as defined in claim 1.

49. A method of inhibiting a fructose-1,6, bisphosphatase (FBPase) in vivo or in vitro comprising contacting a FBPase with a compound according to claim 1 in an amount sufficient to inhibit said FBPase.

50. The method according to claim 43, wherein said disease or condition is selected from hyperlipidemia, atherosclerosis, ischemic injury, hypercholesterolemia, glycogen storage diseases, diabetes, impaired glucose tolerance, insulin resistance, hyperglycemia, obesity, accelerated gluconeogenesis or increased hepatic output.

51. A method of reducing gluconeogenesis in an animal comprising administering to said animal a therapeutically effective amount of the compound of claim 1 or a pharmaceutically acceptable prodrug or salt thereof.
